SEQUENCE LISTING (264 sequences)

SEQ ID NO: 1 - protein, 575 aa, Phaeodactylum tricornutum
MRPYPTIALISVFLSAATRISATSSHQASALPVKKGTHVPDSPKLSKLYIMAKTKSVSSSFDPPRGGSTVAPTTPLATGGALRKVRQAVFPIYGNQEVTKFLLIGSIKFFIILALTLTRDTKDTLIVTQCGAEAIAFLKIYGVLPAATAFIALYSKMSNAMGKKMLFYSTCIPFFTFFGLFDVFIYPNAERLHPSLEAVQAILPGGAASGGMAVLAKIATHWTSALFYVMAEIYSSVSVGLLFWQFANDVVNVDQAKRFYPLFAQMSGLAPVLAGQYVVRFASKAVNFEASMHRLTAAVTFAGIMICIFYQLSSSYVERTESAKPAADNEQSIKPKKKKPKMSMVESGKFLASSQYLRLIAMLVLGYGLSINFTEIMWKSLVKKQYPDPLDYQRFMGNFSSAVGLSTCIVIFFGVHVIRLLGWKVGALATPGIMAILALPFFACILLGLDSPARLEIAVIFGTIQSLLSKTSKYALFDPTTQMAYIPLDDESKVKGKAAIDVLGSRIGKSGGSLIQQGLVFVFGNIINAAPVVGVVYYSVLVAWMSAAGRLSGLFQAQTEMDKADKMEAKTNKEK

SEQ ID NO: 2 - DNA, 1536 bp, synthetic construct
  misc_feature 1..1536: note = Description of Artificial Sequence: Synthetic polynucleotide
  source 1..1536: mol_type = other DNA
atgggtggtagcaccgttgcaccgaccacaccgctggcaaccggtggtgcactgcgtaaagttcgtcaggcagtttttccgatttatggcaatcaagaagtgaccaaatttctgctgattggcagcatcaaattctttattatcctggcactgaccctgacccgtgataccaaagataccctgattgttacccagtgtggtgcagaagcaattgcatttctgaaaatctatggtgttctgcctgcagcaaccgcatttattgcactgtatagcaaaatgagcaacgcaatgggcaaaaaaatgctgttttatagcacctgtatcccgttctttaccttttttggtctgttcgatgtgttcatttatccgaatgccgaacgtctgcatccgagcctggaagcagttcaggcaattctgcctggtggtgccgcaagcggtggtatggcagttctggcaaaaattgcaacccattggaccagcgcactgttttatgttatggcagaaatctatagcagcgttagcgttggtctgctgttttggcagtttgcaaatgatgttgttaatgtggatcaggccaaacgtttttatccgctgtttgcacagatgagcggtctggcaccggttctggcaggtcagtatgttgttcgttttgcaagcaaagccgttaattttgaagcaagcatgcatcgtctgaccgcagcagttacctttgcaggtattatgatctgcatcttttatcagctgagcagctcatatgttgaacgtaccgaaagcgcaaaaccggcagcagataatgaacagagcattaaaccgaagaaaaaaaaaccgaaaatgtcgatggtggaaagcggtaaatttctggcaagcagccagtatctgcgtctgattgcaatgctggttctgggttatggtctgagcattaactttaccgaaatcatgtggaaaagcctggtgaaaaaacagtatccggatccgctggattatcagcgttttatgggtaattttagcagcgcagttggtctgagtacctgcattgttatcttttttggcgtgcatgttattcgtctgctgggttggaaagttggtgccctggcaacaccgggtattatggccattctggcactgccgttttttgcatgtattctgctgggcctggatagtccggcacgtctggaaattgcagttatttttggcaccattcagagcctgctgagcaaaaccagcaaatatgcactgtttgatccgaccacccagatggcatatatcccgctggatgatgaaagcaaagttaaaggcaaagcagccattgatgttctgggtagccgtattggtaaatcaggtggtagcctgattcagcagggtctggtttttgtttttggcaatattatcaatgccgcaccggttgttggtgttgtgtattatagcgttctggttgcatggatgagtgcagcaggtcgtctgagtggtctgtttcaggcacagaccgaaatggataaagcagataaaatggaagccaaaaccaacaaagaaaaatga

SEQ ID NO: 3 - DNA, 1536 bp, Phaeodactylum tricornutum
  source 1..1536: mol_type = other DNA
atgggaggcagtactgttgcaccaactacaccgttggcaaccggcggtgcgctccgcaaagtgcgacaagccgtctttcccatctacggaaaccaagaagtcaccaaatttctgctcatcggatccattaaattctttataatcttggcactcacgctcacgcgtgataccaaggacacgttgattgtcacgcaatgtggtgccgaagcgattgcctttctcaaaatatacggggtgctacccgcagcgaccgcatttatcgcgctctattccaaaatgtccaacgccatgggcaaaaaaatgctattttattccacttgcattcctttctttacctttttcgggctgtttgatgttttcatttacccgaacgcggagcgactgcaccctagtttggaagccgtgcaggcaattctcccgggcggtgccgcatctggcggcatggcggttctggccaagattgcgacacactggacatcggccttattttacgtcatggcggaaatatattcttccgtatcggtggggctattgttttggcagtttgcgaacgacgtcgtcaacgtggatcaggccaagcgcttttatccattatttgctcaaatgagtggcctcgctccagttttagcgggccagtatgtggtacggtttgccagcaaagcggtcaactttgaggcatccatgcatcgactcacggcggccgtaacatttgctggtattatgatttgcatcttttaccaactcagttcgtcatatgtggagcgaacggaatcagcaaagccagcggcagataacgagcagtctatcaaaccgaaaaagaagaaacccaaaatgtccatggttgaatcggggaaatttctcgcgtcaagtcagtacctgcgtctaattgccatgctggtgctgggatacggcctcagtattaactttaccgaaatcatgtggaaaagcttggtgaagaaacaatatccagacccgctagattatcaacgatttatgggtaacttctcgtcagcggttggtttgagcacatgcattgttattttcttcggtgtgcacgtgatccgtttgttggggtggaaagtcggagcgttggctacacctgggatcatggccattctagcgttacccttttttgcttgcattttgttgggtttggatagtccagcacgattggagatcgccgtaatctttggaacaattcagagtttgctgagcaaaacctccaagtatgcccttttcgaccctaccacacaaatggcttatattcctctggacgacgaatcaaaggtcaaaggaaaagcggcaattgatgttttgggatcgcggattggcaagagtggaggctcactgatccagcagggcttggtctttgtttttggaaatatcattaatgccgcacctgtagtaggggttgtctactacagtgtccttgttgcgtggatgagcgcagctggccgactaagtgggctttttcaagcacaaacagaaatggataaggccgacaaaatggaggcaaagaccaacaaagaaaagtag

SEQ ID NO: 4 - protein, 511 aa, Phaeodactylum tricornutum
MGGSTVAPTTPLATGGALRKVRQAVFPIYGNQEVTKFLLIGSIKFFIILALTLTRDTKDTLIVTQCGAEAIAFLKIYGVLPAATAFIALYSKMSNAMGKKMLFYSTCIPFFTFFGLFDVFIYPNAERLHPSLEAVQAILPGGAASGGMAVLAKIATHWTSALFYVMAEIYSSVSVGLLFWQFANDVVNVDQAKRFYPLFAQMSGLAPVLAGQYVVRFASKAVNFEASMHRLTAAVTFAGIMICIFYQLSSSYVERTESAKPAADNEQSIKPKKKKPKMSMVESGKFLASSQYLRLIAMLVLGYGLSINFTEIMWKSLVKKQYPDPLDYQRFMGNFSSAVGLSTCIVIFFGVHVIRLLGWKVGALATPGIMAILALPFFACILLGLDSPARLEIAVIFGTIQSLLSKTSKYALFDPTTQMAYIPLDDESKVKGKAAIDVLGSRIGKSGGSLIQQGLVFVFGNIINAAPVVGVVYYSVLVAWMSAAGRLSGLFQAQTEMDKADKMEAKTNKEK

SEQ ID NO: 5 - DNA, 1599 bp, synthetic construct
  misc_feature 1..1599: note = Description of Artificial Sequence: Synthetic polynucleotide
  source 1..1599: mol_type = other DNA
atgagaccatttccgacgattgccttgatttcggtttttctttcggcggcgactcgcatttcggcaggaggcagtactgttgcaccaactacaccgttggcaaccggcggtgcgctccgcaaagtgcgacaagccgtctttcccatctacggaaaccaagaagtcaccaaatttctgctcatcggatccattaaattctttataatcttggcactcacgctcacgcgtgataccaaggacacgttgattgtcacgcaatgtggtgccgaagcgattgcctttctcaaaatatacggggtgctacccgcagcgaccgcatttatcgcgctctattccaaaatgtccaacgccatgggcaaaaaaatgctattttattccacttgcattcctttctttacctttttcgggctgtttgatgttttcatttacccgaacgcggagcgactgcaccctagtttggaagccgtgcaggcaattctcccgggcggtgccgcatctggcggcatggcggttctggccaagattgcgacacactggacatcggccttattttacgtcatggcggaaatatattcttccgtatcggtggggctattgttttggcagtttgcgaacgacgtcgtcaacgtggatcaggccaagcgcttttatccattatttgctcaaatgagtggcctcgctccagttttagcgggccagtatgtggtacggtttgccagcaaagcggtcaactttgaggcatccatgcatcgactcacggcggccgtaacatttgctggtattatgatttgcatcttttaccaactcagttcgtcatatgtggagcgaacggaatcagcaaagccagcggcagataacgagcagtctatcaaaccgaaaaagaagaaacccaaaatgtccatggttgaatcggggaaatttctcgcgtcaagtcagtacctgcgtctaattgccatgctggtgctgggatacggcctcagtattaactttaccgaaatcatgtggaaaagcttggtgaagaaacaatatccagacccgctagattatcaacgatttatgggtaacttctcgtcagcggttggtttgagcacatgcattgttattttcttcggtgtgcacgtgatccgtttgttggggtggaaagtcggagcgttggctacacctgggatcatggccattctagcgttacccttttttgcttgcattttgttgggtttggatagtccagcacgattggagatcgccgtaatctttggaacaattcagagtttgctgagcaaaacctccaagtatgcccttttcgaccctaccacacaaatggcttatattcctctggacgacgaatcaaaggtcaaaggaaaagcggcaattgatgttttgggatcgcggattggcaagagtggaggctcactgatccagcagggcttggtctttgtttttggaaatatcattaatgccgcacctgtagtaggggttgtctactacagtgtccttgttgcgtggatgagcgcagctggccgactaagtgggctttttcaagcacaaacagaaatggataaggccgacaaaatggaggcaaagaccaacaaagaaaagtag

SEQ ID NO: 6 - protein, 532 aa, synthetic construct
  REGION 1..532: note = Description of Artificial Sequence: Synthetic polypeptide
MRPFPTIALISVFLSAATRISAGGSTVAPTTPLATGGALRKVRQAVFPIYGNQEVTKFLLIGSIKFFIILALTLTRDTKDTLIVTQCGAEAIAFLKIYGVLPAATAFIALYSKMSNAMGKKMLFYSTCIPFFTFFGLFDVFIYPNAERLHPSLEAVQAILPGGAASGGMAVLAKIATHWTSALFYVMAEIYSSVSVGLLFWQFANDVVNVDQAKRFYPLFAQMSGLAPVLAGQYVVRFASKAVNFEASMHRLTAAVTFAGIMICIFYQLSSSYVERTESAKPAADNEQSIKPKKKKPKMSMVESGKFLASSQYLRLIAMLVLGYGLSINFTEIMWKSLVKKQYPDPLDYQRFMGNFSSAVGLSTCIVIFFGVHVIRLLGWKVGALATPGIMAILALPFFACILLGLDSPARLEIAVIFGTIQSLLSKTSKYALFDPTTQMAYIPLDDESKVKGKAAIDVLGSRIGKSGGSLIQQGLVFVFGNIINAAPVVGVVYYSVLVAWMSAAGRLSGLFQAQTEMDKADKMEAKTNKEK

SEQ ID NO: 7 - DNA, 1665 bp, synthetic construct
  misc_feature 1..1665: note = Description of Artificial Sequence: Synthetic polynucleotide
  source 1..1665: mol_type = other DNA
atgacttcctctcatcaagcaagtgcacttcctctcaaaaagggaacgcatgtcccggactctccgaagttgtcaaagctatatatcatggccaaaaccaagagtgtatcctcgtccttcgacccccctcggggaggcagtactgttgcaccaactacaccgttggcaaccggcggtgcgctccgcaaagtgcgacaagccgtctttcccatctacggaaaccaagaagtcaccaaatttctgctcatcggatccattaaattctttataatcttggcactcacgctcacgcgtgataccaaggacacgttgattgtcacgcaatgtggtgccgaagcgattgcctttctcaaaatatacggggtgctacccgcagcgaccgcatttatcgcgctctattccaaaatgtccaacgccatgggcaaaaaaatgctattttattccacttgcattcctttctttacctttttcgggctgtttgatgttttcatttacccgaacgcggagcgactgcaccctagtttggaagccgtgcaggcaattctcccgggcggtgccgcatctggcggcatggcggttctggccaagattgcgacacactggacatcggccttattttacgtcatggcggaaatatattcttccgtatcggtggggctattgttttggcagtttgcgaacgacgtcgtcaacgtggatcaggccaagcgcttttatccattatttgctcaaatgagtggcctcgctccagttttagcgggccagtatgtggtacggtttgccagcaaagcggtcaactttgaggcatccatgcatcgactcacggcggccgtaacatttgctggtattatgatttgcatcttttaccaactcagttcgtcatatgtggagcgaacggaatcagcaaagccagcggcagataacgagcagtctatcaaaccgaaaaagaagaaacccaaaatgtccatggttgaatcggggaaatttctcgcgtcaagtcagtacctgcgtctaattgccatgctggtgctgggatacggcctcagtattaactttaccgaaatcatgtggaaaagcttggtgaagaaacaatatccagacccgctagattatcaacgatttatgggtaacttctcgtcagcggttggtttgagcacatgcattgttattttcttcggtgtgcacgtgatccgtttgttggggtggaaagtcggagcgttggctacacctgggatcatggccattctagcgttacccttttttgcttgcattttgttgggtttggatagtccagcacgattggagatcgccgtaatctttggaacaattcagagtttgctgagcaaaacctccaagtatgcccttttcgaccctaccacacaaatggcttatattcctctggacgacgaatcaaaggtcaaaggaaaagcggcaattgatgttttgggatcgcggattggcaagagtggaggctcactgatccagcagggcttggtctttgtttttggaaatatcattaatgccgcacctgtagtaggggttgtctactacagtgtccttgttgcgtggatgagcgcagctggccgactaagtgggctttttcaagcacaaacagaaatggataaggccgacaaaatggaggcaaagaccaacaaagaaaagtag

SEQ ID NO: 8 - protein, 554 aa, synthetic construct
  REGION 1..554: note = Description of Artificial Sequence: Synthetic polypeptide
MTSSHQASALPLKKGTHVPDSPKLSKLYIMAKTKSVSSSFDPPRGGSTVAPTTPLATGGALRKVRQAVFPIYGNQEVTKFLLIGSIKFFIILALTLTRDTKDTLIVTQCGAEAIAFLKIYGVLPAATAFIALYSKMSNAMGKKMLFYSTCIPFFTFFGLFDVFIYPNAERLHPSLEAVQAILPGGAASGGMAVLAKIATHWTSALFYVMAEIYSSVSVGLLFWQFANDVVNVDQAKRFYPLFAQMSGLAPVLAGQYVVRFASKAVNFEASMHRLTAAVTFAGIMICIFYQLSSSYVERTESAKPAADNEQSIKPKKKKPKMSMVESGKFLASSQYLRLIAMLVLGYGLSINFTEIMWKSLVKKQYPDPLDYQRFMGNFSSAVGLSTCIVIFFGVHVIRLLGWKVGALATPGIMAILALPFFACILLGLDSPARLEIAVIFGTIQSLLSKTSKYALFDPTTQMAYIPLDDESKVKGKAAIDVLGSRIGKSGGSLIQQGLVFVFGNIINAAPVVGVVYYSVLVAWMSAAGRLSGLFQAQTEMDKADKMEAKTNKEK

SEQ ID NO: 9 - DNA, 3013 bp, synthetic construct
  misc_feature 1..3013: note = Description of Artificial Sequence: Synthetic polynucleotide
  misc_difference 2349: note = Any unnatural nucleotide
  source 1..3013: mol_type = other DNA
aacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgaaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttggctttacactttatgcttccggctcgtatgttgtgtggaantgtgagcggataacaatttcacacaggaaacagccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaa

SEQ ID NO: 10 - DNA, 20 bp, synthetic construct
  misc_feature 1..20: note = Description of Artificial Sequence: Synthetic primer
  source 1..20: mol_type = other DNA
gggaggcagtactgttgcac

SEQ ID NO: 11 - DNA, 45 bp, synthetic construct
  misc_feature 1..45: note = Description of Artificial Sequence: Synthetic primer
  source 1..45: mol_type = other DNA
ggtatatctccttattaaagttaaacaaaattatttctacagggg

SEQ ID NO: 12 - DNA, 20 bp, synthetic construct
  misc_feature 1..20: note = Description of Artificial Sequence: Synthetic primer
  source 1..20: mol_type = other DNA
taatacgactcactataggg

SEQ ID NO: 13 - DNA, 19 bp, synthetic construct
  misc_feature 1..19: note = Description of Artificial Sequence: Synthetic primer
  source 1..19: mol_type = other DNA
gctagttattgctcagcgg

SEQ ID NO: 14 - DNA, 22 bp, synthetic construct
  misc_feature 1..22: note = Description of Artificial Sequence: Synthetic primer
  source 1..22: mol_type = other DNA
ttacattaattgcgttgcgctc

SEQ ID NO: 15 - DNA, 31 bp, synthetic construct
  misc_feature 1..31: note = Description of Artificial Sequence: Synthetic primer
  source 1..31: mol_type = other DNA
ttttggcggatggcatttgagaagcacacgg

SEQ ID NO: 16 - DNA, 25 bp, synthetic construct
  misc_feature 1..25: note = Description of Artificial Sequence: Synthetic primer
  source 1..25: mol_type = other DNA
attctcaccaataaaaaacgcccgg

SEQ ID NO: 17 - DNA, 36 bp, synthetic construct
  misc_feature 1..36: note = Description of Artificial Sequence: Synthetic primer
  source 1..36: mol_type = other DNA
cctgtagaaataattttgtttaactttaataaggag

SEQ ID NO: 18 - DNA, 20 bp, synthetic construct
  misc_feature 1..20: note = Description of Artificial Sequence: Synthetic primer
  source 1..20: mol_type = other DNA
ccccgcgcgttggccgattc

SEQ ID NO: 19 - DNA, 19 bp, synthetic construct
  misc_feature 1..19: note = Description of Artificial Sequence: Synthetic primer
  source 1..19: mol_type = other DNA
gaagggcaatcagctgttg

SEQ ID NO: 20 - DNA, 21 bp, synthetic construct
  misc_feature 1..21: note = Description of Artificial Sequence: Synthetic primer
  source 1..21: mol_type = other DNA
cagggcagggtcgttaaatag

SEQ ID NO: 21 - DNA, 52 bp, synthetic construct
  misc_feature 1..52: note = Description of Artificial Sequence: Synthetic primer
  source 1..52: mol_type = other DNA
gacaccatcgaatggcgcaaaacctttcgcggtatggcatgatagcgcccgg

SEQ ID NO: 22 - DNA, 52 bp, synthetic construct
  misc_feature 1..52: note = Description of Artificial Sequence: Synthetic primer
  source 1..52: mol_type = other DNA
ccgggcgctatcatgccataccgcgaaaggttttgcgccattcgatggtgtc

SEQ ID NO: 23 - DNA, 51 bp, synthetic construct
  misc_feature 1..51: note = Description of Artificial Sequence: Synthetic primer
  source 1..51: mol_type = other DNA
atttttctaaatacattcaaatatgtatccgctcatgagacaataaccctg

SEQ ID NO: 24 - DNA, 51 bp, synthetic construct
  misc_feature 1..51: note = Description of Artificial Sequence: Synthetic primer
  source 1..51: mol_type = other DNA
cagggttattgtctcatgagcggatacatatttgaatgtatttagaaaaat

SEQ ID NO: 25 - DNA, 52 bp, synthetic construct
  misc_feature 1..52: note = Description of Artificial Sequence: Synthetic primer
  source 1..52: mol_type = other DNA
ttaggcaccccaggctttacactttatgcttccggctggtatgttgtgtgga

SEQ ID NO: 26 - DNA, 52 bp, synthetic construct
  misc_feature 1..52: note = Description of Artificial Sequence: Synthetic primer
  source 1..52: mol_type = other DNA
tccacacaacataccagccggaagcataaagtgtaaagcctggggtgcctaa

SEQ ID NO: 27 - DNA, 52 bp, synthetic construct
  misc_feature 1..52: note = Description of Artificial Sequence: Synthetic primer
  source 1..52: mol_type = other DNA
ctaggcaccccaggctttacactttatgcttccggctggtataatgtgtgga

SEQ ID NO: 28 - DNA, 52 bp, synthetic construct
  misc_feature 1..52: note = Description of Artificial Sequence: Synthetic primer
  source 1..52: mol_type = other DNA
tccacacattataccagccggaagcataaagtgtaaagcctggggtgcctag

SEQ ID NO: 29 - DNA, 24 bp, synthetic construct
  misc_feature 1..24: note = Description of Artificial Sequence: Synthetic primer
  source 1..24: mol_type = other DNA
ggggataacgcaggaaagaacatg

SEQ ID NO: 30 - DNA, 22 bp, synthetic construct
  misc_feature 1..22: note = Description of Artificial Sequence: Synthetic primer
  source 1..22: mol_type = other DNA
gcacttttcggggaaatgtgcg

SEQ ID NO: 31 - DNA, 75 bp, synthetic construct
  misc_feature 1..75: note = Description of Artificial Sequence: Synthetic primer
  source 1..75: mol_type = other DNA
aattgcggcctatatggatgttggaaccgtaagagaaatagacaggcggtcctgtgacggaagatcacttcgcag

SEQ ID NO: 32 - DNA, 46 bp, synthetic construct
  misc_feature 1..46: note = Description of Artificial Sequence: Synthetic primer
  source 1..46: mol_type = other DNA
tgctcacatgttctttcctgcgttatccccgcgtggtgaaccaggc

SEQ ID NO: 33 - DNA, 28 bp, synthetic construct
  misc_feature 1..28: note = Description of Artificial Sequence: Synthetic primer
  source 1..28: mol_type = other DNA
accgcctgtctatttctcttacggttcc

SEQ ID NO: 34 - DNA, 49 bp, synthetic construct
  misc_feature 1..49: note = Description of Artificial Sequence: Synthetic primer
  source 1..49: mol_type = other DNA
cgcgcttaatgcgccgctacagggcgcgtcgattggtgccagcgcgcag

SEQ ID NO: 35 - DNA, 43 bp, synthetic construct
  misc_feature 1..43: note = Description of Artificial Sequence: Synthetic primer
  source 1..43: mol_type = other DNA
ggtatatctccttattaaagttaaacaaaattatttctacagg

SEQ ID NO: 36 - DNA, 21 bp, synthetic construct
  misc_feature 1..21: note = Description of Artificial Sequence: Synthetic primer
  source 1..21: mol_type = other DNA
cagccacgtttctgcgaaaac

SEQ ID NO: 37 - DNA, 20 bp, synthetic construct
  misc_feature 1..20: note = Description of Artificial Sequence: Synthetic primer
  source 1..20: mol_type = other DNA
tacagcggttccttactggc

SEQ ID NO: 38 - DNA, 26 bp, synthetic construct
  misc_feature 1..26: note = Description of Artificial Sequence: Synthetic primer
  source 1..26: mol_type = other DNA
gggtggtgaatgtgaaaccagtaacg

SEQ ID NO: 39 - DNA, 21 bp, synthetic construct
  misc_feature 1..21: note = Description of Artificial Sequence: Synthetic primer
  source 1..21: mol_type = other DNA
ctgggtgtttacttcggtctg

SEQ ID NO: 40 - DNA, 21 bp, synthetic construct
  misc_feature 1..21: note = Description of Artificial Sequence: Synthetic primer
  source 1..21: mol_type = other DNA
ggccgtaatatccagctgaac

SEQ ID NO: 41 - DNA, 20 bp, synthetic construct
  misc_feature 1..20: note = Description of Artificial Sequence: Synthetic primer
  source 1..20: mol_type = other DNA
actagggtgcagtcgctccg

SEQ ID NO: 42 - DNA, 22 bp, synthetic construct
  misc_feature 1..22: note = Description of Artificial Sequence: Synthetic primer
  source 1..22: mol_type = other DNA
ttaacctaggctgctgccaccg

SEQ ID NO: 43 - DNA, 57 bp, synthetic construct
  misc_feature 1..57: note = Description of Artificial Sequence: Synthetic primer
  source 1..57: mol_type = other DNA
gcgcaacgcaattaatgtaattctgaaatgagctgttgacaattaatcatcggctcg

SEQ ID NO: 44 - DNA, 56 bp, synthetic construct
  misc_feature 1..56: note = Description of Artificial Sequence: Synthetic primer
  source 1..56: mol_type = other DNA
aacaaaattatttctacaggtccacacattatacgagccgatgattaattgtcaac

SEQ ID NO: 45 - DNA, 58 bp, synthetic construct
  misc_feature 1..58: note = Description of Artificial Sequence: Synthetic primer
  source 1..58: mol_type = other DNA
gcgcaacgcaattaatgtaatcataaaaaatttatttgctttcaggaaaatttttctg

SEQ ID NO: 46 - DNA, 59 bp, synthetic construct
  misc_feature 1..59: note = Description of Artificial Sequence: Synthetic primer
  source 1..59: mol_type = other DNA
aacaaaattatttctacaggtgaatctattatacagaaaaattttcctgaaagcaaata

SEQ ID NO: 47 - DNA, 56 bp, synthetic construct
  misc_feature 1..56: note = Description of Artificial Sequence: Synthetic primer
  source 1..56: mol_type = other DNA
gcgcaacgcaattaatgtaattatctctggcggtgttgacataaataccactggcg

SEQ ID NO: 48 - DNA, 57 bp, synthetic construct
  misc_feature 1..57: note = Description of Artificial Sequence: Synthetic primer
  source 1..57: mol_type = other DNA
aacaaaattatttctacaggtgtgctcagtatcaccgccagtggtatttatgtcaac

SEQ ID NO: 49 - DNA, 58 bp, synthetic construct
  misc_feature 1..58: note = Description of Artificial Sequence: Synthetic primer
  source 1..58: mol_type = other DNA
gcgcaacgcaattaatgtaattttaaaaaattcatttgctaaacgcttcaaattctcg

SEQ ID NO: 50 - DNA, 55 bp, synthetic construct
  misc_feature 1..55: note = Description of Artificial Sequence: Synthetic primer
  source 1..55: mol_type = other DNA
aacaaaattatttctacaggtgaagtatattatacgagaatttgaagcgtttagc

SEQ ID NO: 51 - DNA, 21 bp, synthetic construct
  misc_feature 1..21: note = Description of Artificial Sequence: Synthetic primer
  source 1..21: mol_type = other DNA
tggggtgcctaatgagtgagc

SEQ ID NO: 52 - DNA, 26 bp, synthetic construct
  misc_feature 1..26: note = Description of Artificial Sequence: Synthetic primer
  source 1..26: mol_type = other DNA
ctatgaccatgattacgccaagcttg

SEQ ID NO: 53 - DNA, 24 bp, synthetic construct
  misc_feature 1..24: note = Description of Artificial Sequence: Synthetic primer
  source 1..24: mol_type = other DNA
tctcgcggtatcattgcagcactg

SEQ ID NO: 54 - DNA, 18 bp, synthetic construct
  misc_feature 1..18: note = Description of Artificial Sequence: Synthetic primer
  source 1..18: mol_type = other DNA
gccacgctcaccggctcc

SEQ ID NO: 55 - DNA, 21 bp, synthetic construct
  misc_feature 1..21: note = Description of Artificial Sequence: Synthetic primer
  source 1..21: mol_type = other DNA
aacgaaaactcacgttaaggg

SEQ ID NO: 56 - DNA, 17 bp, synthetic construct
  misc_feature 1..17: note = Description of Artificial Sequence: Synthetic primer
  source 1..17: mol_type = other DNA
ccactgagcgtcagacc

SEQ ID NO: 57 - DNA, 30 bp, synthetic construct
  misc_feature 1..30: note = Description of Artificial Sequence: Synthetic primer
  source 1..30: mol_type = other DNA
gagacccgtcgttgacaattaatcatcggc

SEQ ID NO: 58 - DNA, 31 bp, synthetic construct
  misc_feature 1..31: note = Description of Artificial Sequence: Synthetic primer
  source 1..31: mol_type = other DNA
gagaccattctcaccaataaaaaacgcccgg

SEQ ID NO: 59 - DNA, 30 bp, synthetic construct
  misc_feature 1..30: note = Description of Artificial Sequence: Synthetic primer
  source 1..30: mol_type = other DNA
cggggtaccatggacaagaagtactccatt

SEQ ID NO: 60 - DNA, 34 bp, synthetic construct
  misc_feature 1..34: note = Description of Artificial Sequence: Synthetic primer
  source 1..34: mol_type = other DNA
ctagtctagattacaccttcctcttcttcttggg

SEQ ID NO: 61 - DNA, 33 bp, synthetic construct
  misc_feature 1..33: note = Description of Artificial Sequence: Synthetic primer
  source 1..33: mol_type = other DNA
ctccggggaaaccgccgaagccacgcggctcaa

SEQ ID NO: 62 - DNA, 44 bp, synthetic construct
  misc_feature 1..44: note = Description of Artificial Sequence: Synthetic primer
  source 1..44: mol_type = other DNA
cttcggcggtttccccggagtcgaacaggagggcgccaatgagg

SEQ ID NO: 63 - DNA, 40 bp, synthetic construct
  misc_feature 1..40: note = Description of Artificial Sequence: Synthetic primer
  source 1..40: mol_type = other DNA
aggaagaagacgtctcacgcatcttactgcgcagatacgc

SEQ ID NO: 64 - DNA, 60 bp, synthetic construct
  misc_feature 1..60: note = Description of Artificial Sequence: Synthetic primer
  source 1..60: mol_type = other DNA
aagatgcgtgagacgtcttcttcctcgtctcggtcgacagttcataggtgattgctcagg

SEQ ID NO: 65 - DNA, 26 bp, synthetic construct
  misc_feature 1..26: note = Description of Artificial Sequence: Synthetic primer
  source 1..26: mol_type = other DNA
gtcccaaatcgcagccaatcacattg

SEQ ID NO: 66 - DNA, 28 bp, synthetic construct
  misc_feature 1..28: note = Description of Artificial Sequence: Synthetic primer
  source 1..28: mol_type = other DNA
gtcctgaccatcgtattggttatctggc

SEQ ID NO: 67 - DNA, 26 bp, synthetic construct
  misc_feature 1..26: note = Description of Artificial Sequence: Synthetic primer
  source 1..26: mol_type = other DNA
atttagagggcagtgccagctcgtta

SEQ ID NO: 68 - DNA, 26 bp, synthetic construct
  misc_feature 1..26: note = Description of Artificial Sequence: Synthetic primer
  source 1..26: mol_type = other DNA
ctgcattcaggtaggcatcatgcgca

SEQ ID NO: 69 - DNA, 26 bp, synthetic construct
  misc_feature 1..26: note = Description of Artificial Sequence: Synthetic primer
  source 1..26: mol_type = other DNA
ctgggctacctgcaagattagcgatg

SEQ ID NO: 70 - DNA, 25 bp, synthetic construct
  misc_feature 1..25: note = Description of Artificial Sequence: Synthetic primer
  source 1..25: mol_type = other DNA
tgaaggactgggcagaggccccctt

SEQ ID NO: 71 - DNA, 26 bp, synthetic construct
  misc_feature 1..26: note = Description of Artificial Sequence: Synthetic primer
  source 1..26: mol_type = other DNA
cgtaggtgtctttgctcagttgaagc

SEQ ID NO: 72 - DNA, 27 bp, synthetic construct
  misc_feature 1..27: note = Description of Artificial Sequence: Synthetic primer
  source 1..27: mol_type = other DNA
tagccatctcattactaaagatctcct

SEQ ID NO: 73 - DNA, 43 bp, synthetic construct
  misc_feature 1..43: note = Description of Artificial Sequence: Synthetic primer
  source 1..43: mol_type = other DNA
cgatatcgttggtctcaacgacacaattgtaaaggttagatct

SEQ ID NO: 74 - DNA, 43 bp, synthetic construct
  misc_feature 1..43: note = Description of Artificial Sequence: Synthetic primer
  source 1..43: mol_type = other DNA
caacgatatcggtctcacactgactgggcctttcgttttatct

SEQ ID NO: 75 - DNA, 23 bp, synthetic construct
  misc_feature 1..23: note = Description of Artificial Sequence: Synthetic primer
  source 1..23: mol_type = other DNA
gcaatcacctatgaactgtcgac

SEQ ID NO: 76 - DNA, 69 bp, synthetic construct
  misc_feature 1..69: note = Description of Artificial Sequence: Synthetic primer
  source 1..69: mol_type = other DNA
aggaggaaggacgtctcatgcgccccgcattcacacaatgtagtgatcagttttagagctagaaatagc

SEQ ID NO: 77 - DNA, 69 bp, synthetic construct
  misc_feature 1..69: note = Description of Artificial Sequence: Synthetic primer
  source 1..69: mol_type = other DNA
aggaggaaggacgtctcatgcgccccgcattccaggatgggtaccacccgttttagagctagaaatagc

SEQ ID NO: 78 - DNA, 69 bp, synthetic construct
  misc_feature 1..69: note = Description of Artificial Sequence: Synthetic primer
  source 1..69: mol_type = other DNA
aggaggaaggacgtctcatgcgccccgcattcacacaatgtagtcatcagttttagagctagaaatagc

SEQ ID NO: 79 - DNA, 69 bp, synthetic construct
  misc_feature 1..69: note = Description of Artificial Sequence: Synthetic primer
  source 1..69: mol_type = other DNA
aggaggaaggacgtctcatgcgccccgcattccaggatgggcaccagccgttttagagctagaaatagc

SEQ ID NO: 80 - DNA, 69 bp, synthetic construct
  misc_feature 1..69: note = Description of Artificial Sequence: Synthetic primer
  source 1..69: mol_type = other DNA
aggaggaaggacgtctcatgcgccccgcattccatgatgggcaccacccgttttagagctagaaatagc

SEQ ID NO: 81 - DNA, 69 bp, synthetic construct
  misc_feature 1..69: note = Description of Artificial Sequence: Synthetic primer
  source 1..69: mol_type = other DNA
aggaggaaggacgtctcatgcgccccgcattcacacaatgtatagatcagttttagagctagaaatagc

SEQ ID NO: 82 - DNA, 69 bp, synthetic construct
  misc_feature 1..69: note = Description of Artificial Sequence: Synthetic primer
  source 1..69: mol_type = other DNA
aggaggaaggacgtctcatgcgccccgcattccaggatgggcaccatccgttttagagctagaaatagc

SEQ ID NO: 83 - DNA, 69 bp, synthetic construct
  misc_feature 1..69: note = Description of Artificial Sequence: Synthetic primer
  source 1..69: mol_type = other DNA
aggaggaaggacgtctcatgcgccccgcattgttgtgtggaaatgtgaggttttagagctagaaatagc

SEQ ID NO: 84 - DNA, 69 bp, synthetic construct
  misc_feature 1..69: note = Description of Artificial Sequence: Synthetic primer
  source 1..69: mol_type = other DNA
aggaggaaggacgtctcatgcgccccgcatttgtcactactctgaccaggttttagagctagaaatagc

SEQ ID NO: 85 - DNA, 69 bp, synthetic construct
  misc_feature 1..69: note = Description of Artificial Sequence: Synthetic primer
  source 1..69: mol_type = other DNA
aggaggaaggacgtctcatgcgccccgcatttgtcactactctgaccaagttttagagctagaaatagc

SEQ ID NO: 86 - DNA, 69 bp, synthetic construct
  misc_feature 1..69: note = Description of Artificial Sequence: Synthetic primer
  source 1..69: mol_type = other DNA
aggaggaaggacgtctcatgcgccccgcattcacacaatgtactcatcagttttagagctagaaatagc

SEQ ID NO: 87 - DNA, 69 bp, synthetic construct
  misc_feature 1..69: note = Description of Artificial Sequence: Synthetic primer
  source 1..69: mol_type = other DNA
aggaggaaggacgtctcatgcgccccgcattccaagatgggcaccacccgttttagagctagaaatagc

SEQ ID NO: 88 - DNA, 69 bp, synthetic construct
  misc_feature 1..69: note = Description of Artificial Sequence: Synthetic primer
  source 1..69: mol_type = other DNA
aggaggaaggacgtctcatgcgccccgcattcacacaatgtattgatcagttttagagctagaaatagc

SEQ ID NO: 89 - DNA, 69 bp, synthetic construct
  misc_feature 1..69: note = Description of Artificial Sequence: Synthetic primer
  source 1..69: mol_type = other DNA
aggaggaaggacgtctcatgcgccccgcattcacacaatgtataaatcagttttagagctagaaatagc

SEQ ID NO: 90 - DNA, 69 bp, synthetic construct
  misc_feature 1..69: note = Description of Artificial Sequence: Synthetic primer
  source 1..69: mol_type = other DNA
aggaggaaggacgtctcatgcgccccgcattccaggatggggaccacccgttttagagctagaaatagc

SEQ ID NO: 91 - DNA, 69 bp, synthetic construct
  misc_feature 1..69: note = Description of Artificial Sequence: Synthetic primer
  source 1..69: mol_type = other DNA
aggaggaaggacgtctcatgcgccccgcattttcacaatactttctttagttttagagctagaaatagc

SEQ ID NO: 92 - DNA, 43 bp, synthetic construct
  misc_feature 1..43: note = Description of Artificial Sequence: Synthetic primer
  source 1..43: mol_type = other DNA
gcatttcacacaatgtaggatcagttttagagctagaaatagc

SEQ ID NO: 93 - DNA, 38 bp, synthetic construct
  misc_feature 1..38: note = Description of Artificial Sequence: Synthetic primer
  source 1..38: mol_type = other DNA
tgatcctacattgtgtgaaatgcggggcgcatcttact

SEQ ID NO: 94 - DNA, 43 bp, synthetic construct
  misc_feature 1..43: note = Description of Artificial Sequence: Synthetic primer
  source 1..43: mol_type = other DNA
gcattaccaggatgggaccacccgttttagagctagaaatagc

SEQ ID NO: 95 - DNA, 38 bp, synthetic construct
  misc_feature 1..38: note = Description of Artificial Sequence: Synthetic primer
  source 1..38: mol_type = other DNA
gggtggtcccatcctggtaatgcggggcgcatcttact

SEQ ID NO: 96 - DNA, 43 bp, synthetic construct
  misc_feature 1..43: note = Description of Artificial Sequence: Synthetic primer
  source 1..43: mol_type = other DNA
gcatttcacacaatgtagcatcagttttagagctagaaatagc

SEQ ID NO: 97 - DNA, 38 bp, synthetic construct
  misc_feature 1..38: note = Description of Artificial Sequence: Synthetic primer
  source 1..38: mol_type = other DNA
tgatgctacattgtgtgaaatgcggggcgcatcttact

SEQ ID NO: 98 - DNA, 43 bp, synthetic construct
  misc_feature 1..43: note = Description of Artificial Sequence: Synthetic primer
  source 1..43: mol_type = other DNA
gcattaccaggatgggcaccaccgttttagagctagaaatagc

SEQ ID NO: 99 - DNA, 38 bp, synthetic construct
  misc_feature 1..38: note = Description of Artificial Sequence: Synthetic primer
  source 1..38: mol_type = other DNA
ggtggtgcccatcctggtaatgcggggcgcatcttact

SEQ ID NO: 100 - DNA, 43 bp, synthetic construct
  misc_feature 1..43: note = Description of Artificial Sequence: Synthetic primer
  source 1..43: mol_type = other DNA
gcattaccagatgggcaccacccgttttagagctagaaatagc

SEQ ID NO: 101 - DNA, 38 bp, synthetic construct
  misc_feature 1..38: note = Description of Artificial Sequence: Synthetic primer
  source 1..38: mol_type = other DNA
gggtggtgcccatctggtaatgcggggcgcatcttact

SEQ ID NO: 102 - DNA, 43 bp, synthetic construct
  misc_feature 1..43: note = Description of Artificial Sequence: Synthetic primer
  source 1..43: mol_type = other DNA
gcatttcacacaatgtaagatcagttttagagctagaaatagc

SEQ ID NO: 103 - DNA, 38 bp, synthetic construct
  misc_feature 1..38: note = Description of Artificial Sequence: Synthetic primer
  source 1..38: mol_type = other DNA
tgatcttacattgtgtgaaatgcggggcgcatcttact

SEQ ID NO: 104 - DNA, 43 bp, synthetic construct
  misc_feature 1..43: note = Description of Artificial Sequence: Synthetic primer
  source 1..43: mol_type = other DNA
gcatttgttgtgtggaatgtgaggttttagagctagaaatagc

SEQ ID NO: 105 - DNA, 38 bp, synthetic construct
  misc_feature 1..38: note = Description of Artificial Sequence: Synthetic primer
  source 1..38: mol_type = other DNA
ctcacattccacacaacaaatgcggggcgcatcttact

SEQ ID NO: 106 - DNA, 43 bp, synthetic construct
  misc_feature 1..43: note = Description of Artificial Sequence: Synthetic primer
  source 1..43: mol_type = other DNA
gcattttgtcactactctgaccggttttagagctagaaatagc

SEQ ID NO: 107 - DNA, 38 bp, synthetic construct
  misc_feature 1..38: note = Description of Artificial Sequence: Synthetic primer
  source 1..38: mol_type = other DNA
cggtcagagtagtgacaaaatgcggggcgcatcttact

SEQ ID NO: 108 - DNA, 43 bp, synthetic construct
  misc_feature 1..43: note = Description of Artificial Sequence: Synthetic primer
  source 1..43: mol_type = other DNA
gcattttgtcactactctgaccagttttagagctagaaatagc

SEQ ID NO: 109 - DNA, 38 bp, synthetic construct
  misc_feature 1..38: note = Description of Artificial Sequence: Synthetic primer
  source 1..38: mol_type = other DNA
tggtcagagtagtgacaaaatgcggggcgcatcttact

SEQ ID NO: 110 - DNA, 43 bp, synthetic construct
  misc_feature 1..43: note = Description of Artificial Sequence: Synthetic primer
  source 1..43: mol_type = other DNA
gcatttcacacaatgtaccatcagttttagagctagaaatagc

SEQ ID NO: 111 - DNA, 38 bp, synthetic construct
  misc_feature 1..38: note = Description of Artificial Sequence: Synthetic primer
  source 1..38: mol_type = other DNA
tgatggtacattgtgtgaaatgcggggcgcatcttact

SEQ ID NO: 112 - DNA, 43 bp, synthetic construct
  misc_feature 1..43: note = Description of Artificial Sequence: Synthetic primer
  source 1..43: mol_type = other DNA
gcatttcacacaatgtatgatcagttttagagctagaaatagc

SEQ ID NO: 113 - DNA, 38 bp, synthetic construct
  misc_feature 1..38: note = Description of Artificial Sequence: Synthetic primer
  source 1..38: mol_type = other DNA
tgatcatacattgtgtgaaatgcggggcgcatcttact

SEQ ID NO: 114 - DNA, 43 bp, synthetic construct
  misc_feature 1..43: note = Description of Artificial Sequence: Synthetic primer
  source 1..43: mol_type = other DNA
gcatttcacacaatgtataatcagttttagagctagaaatagc

SEQ ID NO: 115 - DNA, 38 bp, synthetic construct
  misc_feature 1..38: note = Description of Artificial Sequence: Synthetic primer
  source 1..38: mol_type = other DNA
tgattatacattgtgtgaaatgcggggcgcatcttact

SEQ ID NO: 116 - DNA, 43 bp, synthetic construct
  misc_feature 1..43: note = Description of Artificial Sequence: Synthetic primer
  source 1..43: mol_type = other DNA
gcattattcacaatacttctttagttttagagctagaaatagc

SEQ ID NO: 117 - DNA, 38 bp, synthetic construct
  misc_feature 1..38: note = Description of Artificial Sequence: Synthetic primer
  source 1..38: mol_type = other DNA
taaagaagtattgtgaataatgcggggcgcatcttact

SEQ ID NO: 118 - DNA, 47 bp, synthetic construct
  misc_feature 1..47: note = Description of Artificial Sequence: Synthetic primer
  source 1..47: mol_type = other DNA
agaaggaagacgtctcactgtcgaccaaaaaagcctgctcgttgagc

SEQ ID NO: 119 - DNA, 40 bp, synthetic construct
  misc_feature 1..40: note = Description of Artificial Sequence: Synthetic primer
  source 1..40: mol_type = other DNA
aagaaggacgtctcaacagtagtggcagcggctaactaag

SEQ ID NO: 120 - DNA, 44 bp, synthetic construct
  misc_feature 1..44: note = Description of Artificial Sequence: Synthetic primer
  source 1..44: mol_type = other DNA
aggagaggacgtctctcgaccaaaaaagcctgctcgttgagcag

SEQ ID NO: 121 - DNA, 59 bp, synthetic construct
  misc_feature 1..59: note = Description of Artificial Sequence: Synthetic primer
  source 1..59: mol_type = other DNA
agtaagatgcgccccgcattgaccaggatgggcaccacccgttttagagctagaaatag

SEQ ID NO: 122 - DNA, 59 bp, synthetic construct
  misc_feature 1..59: note = Description of Artificial Sequence: Synthetic primer
  source 1..59: mol_type = other DNA
ctatttctagctctaaaacgggtggtgcccatcctggtcaatgcggggcgcatcttact

SEQ ID NO: 123 - DNA, 39 bp, synthetic construct
  misc_feature 1..39: note = Description of Artificial Sequence: Synthetic primer
  source 1..39: mol_type = other DNA
gttttagagctagaaatagcaagttaaaataaggctagt

SEQ ID NO: 124 - DNA, 20 bp, synthetic construct
  misc_feature 1..20: note = Description of Artificial Sequence: Synthetic primer
  source 1..20: mol_type = other DNA
aatgcggggcgcatcttact

SEQ ID NO: 125 - DNA, 22 bp, synthetic construct
  misc_feature 1..22: note = Description of Artificial Sequence: Synthetic primer
  source 1..22: mol_type = other DNA
cattggcaccggtctactaaac

SEQ ID NO: 126 - DNA, 22 bp, synthetic construct
  misc_feature 1..22: note = Description of Artificial Sequence: Synthetic primer
  source 1..22: mol_type = other DNA
aaacgtttagtagaccggtgcc

SEQ ID NO: 127 - DNA, 29 bp, synthetic construct
  misc_feature 1..29: note = Description of Artificial Sequence: Synthetic primer
  source 1..29: mol_type = other DNA
cgcattcacacaatgtaagtatcagtttt

SEQ ID NO: 128 - DNA, 29 bp, synthetic construct
  misc_feature 1..29: note = Description of Artificial Sequence: Synthetic primer
  source 1..29: mol_type = other DNA
ctctaaaactgatacttacattgtgtgaa

SEQ ID NO: 129 - DNA, 60 bp, synthetic construct
  misc_feature 1..60: note = Description of Artificial Sequence: Synthetic primer
  source 1..60: mol_type = other DNA
gtttacgtcgccgtccagctcgaccaggatgggcaccaacccggtgaacagctcctcgcc

SEQ ID NO: 130 - DNA, 60 bp, synthetic construct
  misc_feature 1..60: note = Description of Artificial Sequence: Synthetic primer
  source 1..60: mol_type = other DNA
gtttacgtcgccgtccagctcgaccaggatgggcaccagcccggtgaacagctcctcgcc

SEQ ID NO: 131 - DNA, 60 bp, synthetic construct
  misc_feature 1..60: note = Description of Artificial Sequence: Synthetic primer
  source 1..60: mol_type = other DNA
gtttacgtcgccgtccagctcgaccaggatgggcaccatcccggtgaacagctcctcgcc

SEQ ID NO: 132 - DNA, 60 bp, synthetic construct
  misc_feature 1..60: note = Description of Artificial Sequence: Synthetic primer
  source 1..60: mol_type = other DNA
gtttacgtcgccgtccagctcgaccaggatgggcaccaccccggtgaacagctcctcgcc

SEQ ID NO: 133 - DNA, 60 bp, synthetic construct
  misc_feature 1..60: note = Description of Artificial Sequence: Synthetic primer
  source 1..60: mol_type = other DNA
gcatcgccctcgccctcgccggacacgctgaacttgtggccgtttacgtcgccgtccagc

SEQ ID NO: 134 - DNA, 60 bp, synthetic construct
  misc_feature 1..60: note = Description of Artificial Sequence: Synthetic primer
  source 1..60: mol_type = other DNA
tgcagtttcatttgatgctcgatgagttatggtgagcaagggcgaggagctgttcaccgg

SEQ ID NO: 135 - DNA, 79 bp, synthetic construct
  misc_feature 1..79: note = Description of Artificial Sequence: Synthetic primer
  source 1..79: mol_type = other DNA
ttaatacgactcactatagggaccaggatgggcaccaaccgttttagagctagaaatagcaagttaaaataaggctagt

SEQ ID NO: 136 - DNA, 79 bp, synthetic construct
  misc_feature 1..79: note = Description of Artificial Sequence: Synthetic primer
  source 1..79: mol_type = other DNA
ttaatacgactcactatagggaccaggatgggcaccagccgttttagagctagaaatagcaagttaaaataaggctagt

SEQ ID NO: 137 - DNA, 79 bp, synthetic construct
  misc_feature 1..79: note = Description of Artificial Sequence: Synthetic primer
  source 1..79: mol_type = other DNA
ttaatacgactcactatagggaccaggatgggcaccatccgttttagagctagaaatagcaagttaaaataaggctagt

SEQ ID NO: 138 - DNA, 62 bp, synthetic construct
  misc_feature 1..62: note = Description of Artificial Sequence: Synthetic primer
  source 1..62: mol_type = other DNA
ttaatacgactcactatagggaccaggatgggcaccacccgttttagagctatgctgttttg

SEQ ID NO: 139 - DNA, 82 bp, synthetic construct
  misc_feature 1..82: note = Description of Artificial Sequence: Synthetic primer
  source 1..82: mol_type = other DNA
aaaagcaccgactcggtgccactttttcaagttgataacggactagccttattttaacttgctatttctagctctaaaacgg

SEQ ID NO: 140 - DNA, 35 bp, synthetic construct
  misc_feature 1..35: note = Description of Artificial Sequence: Synthetic primer
  source 1..35: mol_type = other DNA
aagaggaagaggttaatacgactcactatagggac

SEQ ID NO: 141 - DNA, 20 bp, synthetic construct
  misc_feature 1..20: note = Description of Artificial Sequence: Synthetic primer
  source 1..20: mol_type = other DNA
aaaagcaccgactcggtgcc

SEQ ID NO: 142 - DNA, 41 bp, synthetic construct
  misc_feature 1..41: note = Description of Artificial Sequence: Synthetic primer
  source 1..41: mol_type = other DNA
actagccttattttaacttgctatttctagctctaaaacgg

SEQ ID NO: 143 - DNA, 48 bp, synthetic construct
  misc_feature 1..48: note = Description of Artificial Sequence: Synthetic primer
  source 1..48: mol_type = other DNA
aagaaggagaaggtctctagtggagcaagggcgaggagctgttcaccg

SEQ ID NO: 144 - DNA, 43 bp, synthetic construct
  misc_feature 1..43: note = Description of Artificial Sequence: Synthetic primer
  source 1..43: mol_type = other DNA
aagagaagagaggtctcatcgtgtttacgtcgccgtccagctc

SEQ ID NO: 145 - DNA, 34 bp, synthetic construct
  misc_feature 1..34: note = Description of Artificial Sequence: Synthetic primer
  source 1..34: mol_type = other DNA
atgggtctccagtggggccaacacttgtcactac

SEQ ID NO: 146 - DNA, 34 bp, synthetic construct
  misc_feature 1..34: note = Description of Artificial Sequence: Synthetic primer
  source 1..34: mol_type = other DNA
atgggtctcttcgtttccggataacgggaaaagc

SEQ ID NO: 147 - DNA, 39 bp, synthetic construct
  misc_feature 1..39: note = Description of Artificial Sequence: Synthetic primer
  source 1..39: mol_type = other DNA
atgggtctccagtggctcgagtacaactttaactcacac

SEQ ID NO: 148 - DNA, 39 bp, synthetic construct
  misc_feature 1..39: note = Description of Artificial Sequence: Synthetic primer
  source 1..39: mol_type = other DNA
atgggtctcttcgttgattccattcttttgtttgtctgc

SEQ ID NO: 149 - DNA, 41 bp, synthetic construct
  misc_feature 1..41: note = Description of Artificial Sequence: Synthetic primer
  source 1..41: mol_type = other DNA
atgggtctctcatagctgtttcctgtgtgaaattgttatcc

SEQ ID NO: 150 - DNA, 37 bp, synthetic construct
  misc_feature 1..37: note = Description of Artificial Sequence: Synthetic primer
  source 1..37: mol_type = other DNA
atgggtctcaccccaggctttacactttatgcttccg

SEQ ID NO: 151 - DNA, 41 bp, synthetic construct
  misc_feature 1..41: note = Description of Artificial Sequence: Synthetic primer
  source 1..41: mol_type = other DNA
atgggtctccagtggctgtttcctgtgtgaaattgttatcc

SEQ ID NO: 152 - DNA, 37 bp, synthetic construct
  misc_feature 1..37: note = Description of Artificial Sequence: Synthetic primer
  source 1..37: mol_type = other DNA
atgggtctcttcgttggctttacactttatgcttccg

SEQ ID NO: 153 - DNA, 36 bp, synthetic construct
  misc_feature 1..36: note = Description of Artificial Sequence: Synthetic primer
  source 1..36: mol_type = other DNA
atgggtctccagtggcacacaggaaacagctatgac

SEQ ID NO: 154 - DNA, 41 bp, synthetic construct
  misc_feature 1..41: note = Description of Artificial Sequence: Synthetic primer
  source 1..41: mol_type = other DNA
atgggtctcttcgttgggttaagcttaactttaagaaggag

SEQ ID NO: 155 - DNA, 40 bp, synthetic construct
  misc_feature 1..40: note = Description of Artificial Sequence: Synthetic primer
  source 1..40: mol_type = other DNA
atgggtctcacacaaactcgagtacaactttaactcacac

SEQ ID NO: 156 - DNA, 33 bp, synthetic construct
  misc_feature 1..33: note = Description of Artificial Sequence: Synthetic primer
  source 1..33: mol_type = other DNA
atgggtctcgattccattcttttgtttgtctgc

SEQ ID NO: 157 - DNA, 29 bp, synthetic construct
  misc_feature 1..29: note = Description of Artificial Sequence: Synthetic primer
  source 1..29: mol_type = other DNA
attggtctcggccgagcggttgaaggcac

SEQ ID NO: 158 - DNA, 29 bp, synthetic construct
  misc_feature 1..29: note = Description of Artificial Sequence: Synthetic primer
  source 1..29: mol_type = other DNA
attggtctctctggaaccctttcgggtcg

SEQ ID NO: 159 - DNA, 19 bp, synthetic construct
  misc_feature 1..19: note = Description of Artificial Sequence: Synthetic primer
  source 1..19: mol_type = other DNA
ggcgaggagctgttcaccg

SEQ ID NO: 160 - DNA, 21 bp, synthetic construct
  misc_feature 1..21: note = Description of Artificial Sequence: Synthetic primer
  source 1..21: mol_type = other DNA
gtttacgtcgccgtccagctc

SEQ ID NO: 161 - DNA, 20 bp, synthetic construct
  misc_feature 1..20: note = Description of Artificial Sequence: Synthetic primer
  source 1..20: mol_type = other DNA
ggccaacacttgtcactact

SEQ ID NO: 162 - DNA, 19 bp, synthetic construct
  misc_feature 1..19: note = Description of Artificial Sequence: Synthetic primer
  source 1..19: mol_type = other DNA
tccggataacgggaaaagc

SEQ ID NO: 163 - DNA, 24 bp, synthetic construct
  misc_feature 1..24: note = Description of Artificial Sequence: Synthetic primer
  source 1..24: mol_type = other DNA
ctcgagtacaactttaactcacac

SEQ ID NO: 164 - DNA, 24 bp, synthetic construct
  misc_feature 1..24: note = Description of Artificial Sequence: Synthetic primer
  source 1..24: mol_type = other DNA
gattccattcttttgtttgtctgc

SEQ ID NO: 165 - DNA, 26 bp, synthetic construct
  misc_feature 1..26: note = Description of Artificial Sequence: Synthetic primer
  source 1..26: mol_type = other DNA
ctgtttcctgtgtgaaattgttatcc

SEQ ID NO: 166 - DNA, 22 bp, synthetic construct
  misc_feature 1..22: note = Description of Artificial Sequence: Synthetic primer
  source 1..22: mol_type = other DNA
ggctttacactttatgcttccg

SEQ ID NO: 167 - DNA, 26 bp, synthetic construct
  misc_feature 1..26: note = Description of Artificial Sequence: Synthetic primer
  source 1..26: mol_type = other DNA
cccgggttattacatgcgctagcact

SEQ ID NO: 168 - DNA, 48 bp, synthetic construct
  misc_feature 1..48: note = Description of Artificial Sequence: Synthetic primer
  source 1..48: mol_type = other DNA
gaaattaatacgactcactatagggttaagcttaactttaagaaggag

SEQ ID NO: 169 - DNA, 21 bp, synthetic construct
  misc_feature 1..21: note = Description of Artificial Sequence: Synthetic primer
  source 1..21: mol_type = other DNA
tgcaagcagcagattacgcgc

SEQ ID NO: 170 - DNA, 24 bp, synthetic construct
  misc_feature 1..24: note = Description of Artificial Sequence: Synthetic primer
  source 1..24: mol_type = other DNA
gtaactgtcagaccaagtttactc

SEQ ID NO: 171 - DNA, 60 bp, synthetic construct
  misc_feature 1..60: note = Description of Artificial Sequence: Synthetic oligonucleotide
  modified_base 22: mod_base = OTHER; note = (1R)-1,4-Anhydro-2-deoxy-1-(3-methoxynaphthalen-2-y l)-D-erythro-pentitol
  source 1..60: mol_type = other DNA
ggcgaggagctgttcaccgggntggtgcccatcctggtcgagctggacggcgacgtaaac

SEQ ID NO: 172 - DNA, 60 bp, synthetic construct
  misc_feature 1..60: note = Description of Artificial Sequence: Synthetic oligonucleotide
  modified_base 34: mod_base = OTHER; note = (1R)-1,4-Anhydro-2-deoxy-1-(3-methoxynaphthalen-2-y l)-D-erythro-pentitol
  source 1..60: mol_type = other DNA
gtttacgtcgccgtccagctcgaccaggatgggnaccaccccggtgaacagctcctcgcc

SEQ ID NO: 173 - DNA, 60 bp, synthetic construct
  misc_feature 1..60: note = Description of Artificial Sequence: Synthetic oligonucleotide
  modified_base 27: mod_base = OTHER; note = (1R)-1,4-Anhydro-2-deoxy-1-(3-methoxynaphthalen-2-y l)-D-erythro-pentitol
  source 1..60: mol_type = other DNA
gtttacgtcgccgtccagctcgaccangatgggcaccaccccggtgaacagctcctcgcc

SEQ ID NO: 174 - DNA, 60 bp, synthetic construct
  misc_feature 1..60: note = Description of Artificial Sequence: Synthetic oligonucleotide
  modified_base 39: mod_base = OTHER; note = (1R)-1,4-Anhydro-2-deoxy-1-(3-methoxynaphthalen-2-y l)-D-erythro-pentitol
  source 1..60: mol_type = other DNA
gtttacgtcgccgtccagctcgaccaggatgggcaccancccggtgaacagctcctcgcc

SEQ ID NO: 175 - DNA, 75 bp, synthetic construct
  misc_feature 1..75: note = Description of Artificial Sequence: Synthetic oligonucleotide
  modified_base 34: mod_base = OTHER; note = (1R)-1,4-Anhydro-2-deoxy-1-(3-methoxynaphthalen-2-y l)-D-erythro-pentitol
  source 1..75: mol_type = other DNA
ctgtttcctgtgtgaaattgttatccgctcacanttccacacaacatacgagccggaagcataaagtgtaaagcc

SEQ ID NO: 176 - DNA, 63 bp, synthetic construct
  misc_feature 1..63: note = Description of Artificial Sequence: Synthetic oligonucleotide
  modified_base 31: mod_base = OTHER; note = (1R)-1,4-Anhydro-2-deoxy-1-(3-methoxynaphthalen-2-y l)-D-erythro-pentitol
  source 1..63: mol_type = other DNA
ctcgagtacaactttaactcacacaatgtanagatcacggcagacaaacaaaagaatggaatc

SEQ ID NO: 177 - DNA, 58 bp, synthetic construct
  misc_feature 1..58: note = Description of Artificial Sequence: Synthetic oligonucleotide
  modified_base 31: mod_base = OTHER; note = (1R)-1,4-Anhydro-2-deoxy-1-(3-methoxynaphthalen-2-y l)-D-erythro-pentitol
  source 1..58: mol_type = other DNA
ccggataacgggaaaagcattgaacaccgcnggtcagagtagtgacaagtgttggcca

SEQ ID NO: 178 - DNA, 58 bp, synthetic construct
  misc_feature 1..58: note = Description of Artificial Sequence: Synthetic oligonucleotide
  modified_base 27: mod_base = OTHER; note = (1R)-1,4-Anhydro-2-deoxy-1-(3-methoxynaphthalen-2-y l)-D-erythro-pentitol
  source 1..58: mol_type = other DNA
ggccaacacttgtcactactctgaccnagggtgttcaatgcttttcccgttatccgga

SEQ ID NO: 179 - DNA, 60 bp, synthetic construct
  misc_feature 1..60: note = Description of Artificial Sequence: Synthetic oligonucleotide
  modified_base 34: mod_base = OTHER; note = (1R)-1,4-Anhydro-2-deoxy-1-(3-methoxynaphthalen-2-y l)-D-erythro-pentitol
  source 1..60: mol_type = other DNA
ggcgaggagctgttcaccggggtggtgcccatcntggtcgagctggacggcgacgtaaac

SEQ ID NO: 180 - DNA, 60 bp, synthetic construct
  misc_feature 1..60: note = Description of Artificial Sequence: Synthetic oligonucleotide
  modified_base 27: mod_base = OTHER; note = (1R)-1,4-Anhydro-2-deoxy-1-(3-methoxynaphthalen-2-y l)-D-erythro-pentitol
  source 1..60: mol_type = other DNA
ggcgaggagctgttcaccggggtggtncccatcctggtcgagctggacggcgacgtaaac

SEQ ID NO: 181 - DNA, 64 bp, synthetic construct
  misc_feature 1..64: note = Description of Artificial Sequence: Synthetic oligonucleotide
  modified_base 32: mod_base = OTHER; note = (1R)-1,4-Anhydro-2-deoxy-1-(3-methoxynaphthalen-2-y l)-D-erythro-pentitol
  source 1..64: mol_type = other DNA
gattccattcttttgtttgtctgccgtgattnatacattgtgtgagttaaagttgtactcgagt

SEQ ID NO: 182 - DNA, 134 bp, synthetic construct
  misc_feature 1..134: note = Description of Artificial Sequence: Synthetic polynucleotide
  modified_base 63: mod_base = OTHER; note = (1R)-1,4-Anhydro-2-deoxy-1-(3-methoxynaphthalen-2-y l)-D-erythro-pentitol
  source 1..134: mol_type = other DNA
cacacaggaaacagctatgacccgggttattacatgcgctagcacttggaattcacaatactntctttaaggaaaccatagtaaatctccttcttaaagttaagcttaaccctatagtgagtcgtattaatttc

SEQ ID NO: 183 - DNA, 63 bp, synthetic construct
  misc_feature 1..63: note = Description of Artificial Sequence: Synthetic oligonucleotide
  modified_base 32: mod_base = OTHER; note = (1R)-1,4-Anhydro-2-deoxy-1-(3-methoxynaphthalen-2-y l)-D-erythro-pentitol
  source 1..63: mol_type = other DNA
ctcgagtacaactttaactcacacaatgtaanaatcacggcagacaaacaaaagaatggaatc

SEQ ID NO: 184 - DNA, 63 bp, synthetic construct
  misc_feature 1..63: note = Description of Artificial Sequence: Synthetic oligonucleotide
  modified_base 32: mod_base = OTHER; note = (1R)-1,4-Anhydro-2-deoxy-1-(3-methoxynaphthalen-2-y l)-D-erythro-pentitol
  source 1..63: mol_type = other DNA
ctcgagtacaactttaactcacacaatgtaancatcacggcagacaaacaaaagaatggaatc

SEQ ID NO: 185 - DNA, 63 bp, synthetic construct
  misc_feature 1..63: note = Description of Artificial Sequence: Synthetic oligonucleotide
  modified_base 32: mod_base = OTHER; note = (1R)-1,4-Anhydro-2-deoxy-1-(3-methoxynaphthalen-2-y l)-D-erythro-pentitol
  source 1..63: mol_type = other DNA
ctcgagtacaactttaactcacacaatgtaangatcacggcagacaaacaaaagaatggaatc

SEQ ID NO: 186 - DNA, 63 bp, synthetic construct
  misc_feature 1..63: note = Description of Artificial Sequence: Synthetic oligonucleotide
  modified_base 32: mod_base = OTHER; note = (1R)-1,4-Anhydro-2-deoxy-1-(3-methoxynaphthalen-2-y l)-D-erythro-pentitol
  source 1..63: mol_type = other DNA
ctcgagtacaactttaactcacacaatgtcantgtcacggcagacaaacaaaagaatggaatc

SEQ ID NO: 187 - DNA, 63 bp, synthetic construct
  misc_feature 1..63: note = Description of Artificial Sequence: Synthetic oligonucleotide
  modified_base 32: mod_base = OTHER; note = (1R)-1,4-Anhydro-2-deoxy-1-(3-methoxynaphthalen-2-y l)-D-erythro-pentitol
  source 1..63: mol_type = other DNA
ctcgagtacaactttaactcacacaatgtacnaatcacggcagacaaacaaaagaatggaatc

SEQ ID NO: 188 - DNA, 63 bp, synthetic construct
  misc_feature 1..63: note = Description of Artificial Sequence: Synthetic oligonucleotide
  modified_base 32: mod_base = OTHER; note = (1R)-1,4-Anhydro-2-deoxy-1-(3-methoxynaphthalen-2-y l)-D-erythro-pentitol
  source 1..63: mol_type = other DNA
ctcgagtacaactttaactcacacaatgtacncatcacggcagacaaacaaaagaatggaatc

SEQ ID NO: 189 - DNA, 63 bp, synthetic construct
  misc_feature 1..63: note = Description of Artificial Sequence: Synthetic oligonucleotide
  modified_base 32: mod_base = OTHER; note = (1R)-1,4-Anhydro-2-deoxy-1-(3-methoxynaphthalen-2-y l)-D-erythro-pentitol
  source 1..63: mol_type = other DNA
ctcgagtacaactttaactcacacaatgtacngatcacggcagacaaacaaaagaatggaatc

SEQ ID NO: 190 - DNA, 63 bp, synthetic construct
  misc_feature 1..63: note = Description of Artificial Sequence: Synthetic oligonucleotide
  modified_base 32: mod_base = OTHER; note = (1R)-1,4-Anhydro-2-deoxy-1-(3-methoxynaphthalen-2-y l)-D-erythro-pentitol
  source 1..63: mol_type = other DNA
ctcgagtacaactttaactcacacaatgtacntatcacggcagacaaacaaaagaatggaatc

SEQ ID NO: 191 - DNA, 63 bp, synthetic construct
  misc_feature 1..63: note = Description of Artificial Sequence: Synthetic oligonucleotide
  modified_base 32: mod_base = OTHER; note = (1R)-1,4-Anhydro-2-deoxy-1-(3-methoxynaphthalen-2-y l)-D-erythro-pentitol
  source 1..63: mol_type = other DNA
ctcgagtacaactttaactcacacaatgtagnaatcacggcagacaaacaaaagaatggaatc

SEQ ID NO: 192 - DNA, 63 bp, synthetic construct
  misc_feature 1..63: note = Description of Artificial Sequence: Synthetic oligonucleotide
  modified_base 32: mod_base = OTHER; note = (1R)-1,4-Anhydro-2-deoxy-1-(3-methoxynaphthalen-2-y l)-D-erythro-pentitol
  source 1..63: mol_type = other DNA
ctcgagtacaactttaactcacacaatgtagncatcacggcagacaaacaaaagaatggaatc

SEQ ID NO: 193 - DNA, 63 bp, synthetic construct
  misc_feature 1..63: note = Description of Artificial Sequence: Synthetic oligonucleotide
  modified_base 32: mod_base = OTHER; note = (1R)-1,4-Anhydro-2-deoxy-1-(3-methoxynaphthalen-2-y l)-D-erythro-pentitol
  source 1..63: mol_type = other DNA
ctcgagtacaactttaactcacacaatgtagngatcacggcagacaaacaaaagaatggaatc

SEQ ID NO: 194 - DNA, 63 bp, synthetic construct
  misc_feature 1..63: note = Description of Artificial Sequence: Synthetic oligonucleotide
  modified_base 32: mod_base = OTHER; note = (1R)-1,4-Anhydro-2-deoxy-1-(3-methoxynaphthalen-2-y l)-D-erythro-pentitol
  source 1..63: mol_type = other DNA
ctcgagtacaactttaactcacacaatgtagntatcacggcagacaaacaaaagaatggaatc

SEQ ID NO: 195 - DNA, 63 bp, synthetic construct
  misc_feature 1..63: note = Description of Artificial Sequence: Synthetic oligonucleotide
  modified_base 32: mod_base = OTHER; note = (1R)-1,4-Anhydro-2-deoxy-1-(3-methoxynaphthalen-2-y l)-D-erythro-pentitol
  source 1..63: mol_type = other DNA
ctcgagtacaactttaactcacacaatgtatnaatcacggcagacaaacaaaagaatggaatc

SEQ ID NO: 196 - DNA, 63 bp, synthetic construct
  misc_feature 1..63: note = Description of Artificial Sequence: Synthetic oligonucleotide
  modified_base 32: mod_base = OTHER; note = (1R)-1,4-Anhydro-2-deoxy-1-(3-methoxynaphthalen-2-y l)-D-erythro-pentitol
  source 1..63: mol_type = other DNA
ctcgagtacaactttaactcacacaatgtatncatcacggcagacaaacaaaagaatggaatc

SEQ ID NO: 197 - DNA, 63 bp, synthetic construct
  misc_feature 1..63: note = Description of Artificial Sequence: Synthetic oligonucleotide
  modified_base 32: mod_base = OTHER; note = (1R)-1,4-Anhydro-2-deoxy-1-(3-methoxynaphthalen-2-y l)-D-erythro-pentitol
  source 1..63: mol_type = other DNA
ctcgagtacaactttaactcacacaatgtatngatcacggcagacaaacaaaagaatggaatc

SEQ ID NO: 198 - DNA, 63 bp, synthetic construct
  misc_feature 1..63: note = Description of Artificial Sequence: Synthetic oligonucleotide
  modified_base 32: mod_base = OTHER; note = (1R)-1,4-Anhydro-2-deoxy-1-(3-methoxynaphthalen-2-y l)-D-erythro-pentitol
  source 1..63: mol_type = other DNA
ctcgagtacaactttaactcacacaatgtatntatcacggcagacaaacaaaagaatggaatc

SEQ ID NO: 199 - DNA, 63 bp, synthetic construct
  misc_feature 1..63: note = Description of Artificial Sequence: Synthetic oligonucleotide
  modified_base 33: mod_base = OTHER; note = (1R)-1,4-Anhydro-2-deoxy-1-(3-methoxynaphthalen-2-y l)-D-erythro-pentitol
  source 1..63: mol_type = other DNA
ctcgagtacaactttaactcacacaatgtaagnatcacggcagacaaacaaaagaatggaatc

SEQ ID NO: 200 - DNA, 56 bp, synthetic construct
  misc_feature 1..56: note = Description of Artificial Sequence: Synthetic oligonucleotide
  modified_base 31: mod_base = OTHER; note = (1R)-1,4-Anhydro-2-deoxy-1-(3-methoxynaphthalen-2-y l)-D-erythro-pentitol
  source 1..56: mol_type = other DNA
ctctggaaccctttcgggtcgccggtttagnagaccggtgccttcaaccgctcggc

SEQ ID NO: 201 - DNA, 23 bp, synthetic construct
  misc_feature 1..23: note = Description of Artificial Sequence: Synthetic oligonucleotide
  misc_difference 18: note = n = dC, dT, dA, dG, [(2-deoxy-beta-D-erythro-pentof uranosyl)-thieno[3,4]pyridine-2-thione], or (1R)-1,4-Anhyd ro-2-deoxy-1-(3-methoxynaphthalen-2-yl)-D-erythro-pentitol
  misc_feature 1..23: note = See specification as filed for detailed description of substitutions and preferred embodiments
  source 1..23: mol_type = other DNA
ctggtcctacccgtggtnggtcc

SEQ ID NO: 202 - DNA, 20 bp, synthetic construct
  misc_feature 19..20: note = DNA
  misc_feature 1..20: note = Description of Combined DNA/RNA Molecule: Synthetic oligonucleotide
  misc_feature 1..20: note = See specification as filed for detailed description of substitutions and preferred embodiments
  source 1..20: mol_type = other DNA
  misc_feature 1..17: note = DNA
  misc_feature 18: note = RNA
gaccaggatgggcaccancc

SEQ ID NO: 203 - RNA, 20 nt, synthetic construct
  misc_feature 1..20: note = Description of Artificial Sequence: Synthetic oligonucleotide
  source 1..20: mol_type = other RNA
gaccaggatgggcaccaccc

SEQ ID NO: 204 - RNA, 18 nt, synthetic construct
  misc_feature 1..18: note = Description of Artificial Sequence: Synthetic oligonucleotide
  source 1..18: mol_type = other RNA
gttgtgtggaaatgtgag

SEQ ID NO: 205 - RNA, 18 nt, synthetic construct
  misc_feature 1..18: note = Description of Artificial Sequence: Synthetic oligonucleotide
  source 1..18: mol_type = other RNA
tgttgtgtggaatgtgag

SEQ ID NO: 206 - DNA, 22 bp, synthetic construct
  misc_feature 1..22: note = Description of Combined DNA/RNA Molecule: Synthetic oligonucleotide
  modified_base 16: mod_base = OTHER; note = [(2-deoxy-beta-D-erythro-pentofuranosyl)-thieno[3,4 ]pyridine-2-thione]
  source 1..22: mol_type = other DNA
  misc_feature 2: note = RNA
  misc_feature 1: note = DNA
  misc_feature 3..22: note = DNA
gtatgttgtgtggaantgtgag

SEQ ID NO: 207 - DNA, 13 bp, synthetic construct
  misc_feature 1..13: note = Description of Artificial Sequence: Synthetic oligonucleotide
  source 1..13: mol_type = other DNA
  CDS 1..12
tcacatttccaca

SEQ ID NO: 208 - protein, 4 aa, synthetic construct
  REGION 1..4: note = Description of Artificial Sequence: Synthetic peptide
SHFH

SEQ ID NO: 209 - DNA, 22 bp, synthetic construct
  misc_feature 1..22: note = Description of Artificial Sequence: Synthetic oligonucleotide
  modified_base 14: mod_base = OTHER; note = (1R)-1,4-Anhydro-2-deoxy-1-(3-methoxynaphthalen-2-y l)-D-erythro-pentitol
  source 1..22: mol_type = other DNA
tcacacaatgtagngatcacgg

SEQ ID NO: 210 - DNA, 22 bp, synthetic construct
  misc_feature 1..22: note = Description of Artificial Sequence: Synthetic oligonucleotide
  modified_base 12: mod_base = OTHER; note = (1R)-1,4-Anhydro-2-deoxy-1-(3-methoxynaphthalen-2-y l)-D-erythro-pentitol
  source 1..22: mol_type = other DNA
accaggatgggnaccaccccgg

SEQ ID NO: 211 - DNA, 22 bp, synthetic construct
  misc_feature 1..22: note = Description of Artificial Sequence: Synthetic oligonucleotide
  modified_base 14: mod_base = OTHER; note = (1R)-1,4-Anhydro-2-deoxy-1-(3-methoxynaphthalen-2-y l)-D-erythro-pentitol
  source 1..22: mol_type = other DNA
tcacacaatgtagncatcacgg

SEQ ID NO: 212 - DNA, 22 bp, synthetic construct
  misc_feature 1..22: note = Description of Artificial Sequence: Synthetic oligonucleotide
  modified_base 17: mod_base = OTHER; note = [(2-deoxy-beta-D-erythro-pentofuranosyl)-thieno[3,4 ]pyridine-2-thione]
  source 1..22: mol_type = other DNA
accaggatgggcaccancccgg

SEQ ID NO: 213 - DNA, 22 bp, synthetic construct
  misc_feature 1..22: note = Description of Artificial Sequence: Synthetic oligonucleotide
  modified_base 5: mod_base = OTHER; note = (1R)-1,4-Anhydro-2-deoxy-1-(3-methoxynaphthalen-2-y l)-D-erythro-pentitol
  source 1..22: mol_type = other DNA
accangatgggcaccaccccgg

SEQ ID NO: 214 - DNA, 22 bp, synthetic construct
  misc_feature 1..22: note = Description of Artificial Sequence: Synthetic oligonucleotide
  modified_base 13: mod_base = OTHER; note = (1R)-1,4-Anhydro-2-deoxy-1-(3-methoxynaphthalen-2-y l)-D-erythro-pentitol
  source 1..22: mol_type = other DNA
tcacacaatgtanagatcacgg

SEQ ID NO: 215 - DNA, 22 bp, synthetic construct
  misc_feature 1..22: note = Description of Artificial Sequence: Synthetic oligonucleotide
  modified_base 17: mod_base = OTHER; note = (1R)-1,4-Anhydro-2-deoxy-1-(3-methoxynaphthalen-2-y l)-D-erythro-pentitol
  source 1..22: mol_type = other DNA
accaggatgggcaccancccgg

SEQ ID NO: 216 - DNA, 22 bp, synthetic construct
  misc_feature 1..22: note = Description of Artificial Sequence: Synthetic oligonucleotide
  modified_base 13: mod_base = OTHER; note = [(2-deoxy-beta-D-erythro-pentofuranosyl)-thieno[3,4 ]pyridine-2-thione]
  source 1..22: mol_type = other DNA
tgttgtgtggaantgtgagcgg

SEQ ID NO: 217 - DNA, 22 bp, synthetic construct
  misc_feature 1..22: note = Description of Artificial Sequence: Synthetic oligonucleotide
  modified_base 18: mod_base = OTHER; note = [(2-deoxy-beta-D-erythro-pentofuranosyl)-thieno[3,4 ]pyridine-2-thione]
  source 1..22: mol_type = other DNA
ttgtcactactctgaccngcgg

SEQ ID NO: 218 - DNA, 22 bp, synthetic construct
  misc_feature 1..22: note = Description of Artificial Sequence: Synthetic oligonucleotide
  modified_base 18: mod_base = OTHER; note = (1R)-1,4-Anhydro-2-deoxy-1-(3-methoxynaphthalen-2-y l)-D-erythro-pentitol
  source 1..22: mol_type = other DNA
ttgtcactactctgaccnaggg

SEQ ID NO: 219 - DNA, 22 bp, synthetic construct
  misc_feature 1..22: note = Description of Artificial Sequence: Synthetic oligonucleotide
  modified_base 14: mod_base = OTHER; note = (1R)-1,4-Anhydro-2-deoxy-1-(3-methoxynaphthalen-2-y l)-D-erythro-pentitol
  source 1..22: mol_type = other DNA
tcacacaatgtacncatcacgg

SEQ ID NO: 220 - DNA, 22 bp, synthetic construct
  misc_feature 1..22: note = Description of Artificial Sequence: Synthetic oligonucleotide
  modified_base 5: mod_base = OTHER; note = [(2-deoxy-beta-D-erythro-pentofuranosyl)-thieno[3,4 ]pyridine-2-thione]
  source 1..22: mol_type = other DNA
accangatgggcaccaccccgg

SEQ ID NO: 221 - DNA, 22 bp, synthetic construct
  misc_feature 1..22: note = Description of Artificial Sequence: Synthetic oligonucleotide
  modified_base 14: mod_base = OTHER; note = (1R)-1,4-Anhydro-2-deoxy-1-(3-methoxynaphthalen-2-y l)-D-erythro-pentitol
  source 1..22: mol_type = other DNA
tcacacaatgtatngatcacgg

SEQ ID NO: 222 - DNA, 22 bp, synthetic construct
  misc_feature 1..22: note = Description of Artificial Sequence: Synthetic oligonucleotide
  modified_base 14: mod_base = OTHER; note = [(2-deoxy-beta-D-erythro-pentofuranosyl)-thieno[3,4 ]pyridine-2-thione]
  source 1..22: mol_type = other DNA
tcacacaatgtatnaatcacgg

SEQ ID NO: 223 - DNA, 22 bp, synthetic construct
  misc_feature 1..22: note = Description of Artificial Sequence: Synthetic oligonucleotide
  modified_base 12: mod_base = OTHER; note = [(2-deoxy-beta-D-erythro-pentofuranosyl)-thieno[3,4 ]pyridine-2-thione]
  source 1..22: mol_type = other DNA
accaggatgggnaccaccccgg

SEQ ID NO: 224 - DNA, 22 bp, synthetic construct
  misc_feature 1..22: note = Description of Artificial Sequence: Synthetic oligonucleotide
  modified_base 13: mod_base = OTHER; note = (1R)-1,4-Anhydro-2-deoxy-1-(3-methoxynaphthalen-2-y l)-D-erythro-pentitol
  source 1..22: mol_type = other DNA
attcacaatactntctttaagg

SEQ ID NO: 225 - protein, 22 aa, synthetic construct
  REGION 1..22: note = Description of Artificial Sequence: Synthetic peptide
MKYLLPTAEAGLLLLAAQPAIA

SEQ ID NO: 226 - protein, 26 aa, synthetic construct
  REGION 1..26: note = Description of Artificial Sequence: Synthetic peptide
MKIKTGARILALSELTTMMFSASALA

SEQ ID NO: 227 - protein, 21 aa, synthetic construct
  REGION 1..21: note = Description of Artificial Sequence: Synthetic peptide
MKQSTIALALLPLLFTPVTKA

SEQ ID NO: 228 - protein, 30 aa, synthetic construct
  REGION 1..30: note = Description of Artificial Sequence: Synthetic polypeptide
MKSPAPSRPQKMALIPACIFLCFAALSVQA

SEQ ID NO: 229 - protein, 20 aa, synthetic construct
  REGION 1..20: note = Description of Artificial Sequence: Synthetic peptide
MKKILVSFVAIMAVASSAMA

SEQ ID NO: 230 - protein, 40 aa, synthetic construct
  REGION 1..40: note = Description of Artificial Sequence: Synthetic polypeptide
MKISMQKADFWKKAAISLLVFTMFFTLMMSETVFAAGLNK

SEQ ID NO: 231 - protein, 28 aa, synthetic construct
  REGION 1..28: note = Description of Artificial Sequence: Synthetic peptide
MKKTAIAIAVALAGFATVAQASAGLNKD

SEQ ID NO: 232 - protein, 19 aa, synthetic construct
  REGION 1..19: note = Description of Artificial Sequence: Synthetic peptide
MKKIWLALAGLVLAFSASA

SEQ ID NO: 233 - DNA, 57 bp, synthetic construct
  misc_feature 1..57: note = Description of Artificial Sequence: Synthetic oligonucleotide
  source 1..57: mol_type = other DNA
atgaaaaagatttggctggcgctggctggtttagttttagcgtttagcgcatcggcg

SEQ ID NO: 234 - protein, 22 aa, synthetic construct
  REGION 1..22: note = Description of Artificial Sequence: Synthetic peptide
MKYLLPTAAAGLLLLAAQPAMA

SEQ ID NO: 235 - DNA, 66 bp, synthetic construct
  misc_feature 1..66: note = Description of Artificial Sequence: Synthetic oligonucleotide
  source 1..66: mol_type = other DNA
atgaaatacctgctgccgaccgctgctgctggtctgctgctcctcgctgcccagccggcgatggcg

SEQ ID NO: 236 - DNA, 63 bp, synthetic construct
  misc_feature 1..63: note = Description of Artificial Sequence: Synthetic oligonucleotide
  source 1..63: mol_type = other DNA
atgaaacaaagcactattgcactggcactcttaccgttactgtttacccctgtgacaaaagcg

SEQ ID NO: 237 - protein, 26 aa, synthetic construct
  REGION 1..26: note = Description of Artificial Sequence: Synthetic peptide
MKTHIVSSVTTTLLLGSILMNPVANA

SEQ ID NO: 238 - DNA, 78 bp, synthetic construct
  misc_feature 1..78: note = Description of Artificial Sequence: Synthetic oligonucleotide
  source 1..78: mol_type = other DNA
atgaaaacacatatagtcagctcagtaacaacaacactattgctaggttccatattaatgaatcctgtcgctaatgcc

SEQ ID NO: 239 - protein, 21 aa, synthetic construct
  REGION 1..21: note = Description of Artificial Sequence: Synthetic peptide
MKYLLPWLALAGLVLAFSASA

SEQ ID NO: 240 - DNA, 63 bp, synthetic construct
  misc_feature 1..63: note = Description of Artificial Sequence: Synthetic oligonucleotide
  source 1..63: mol_type = other DNA
atgaaatacctgctgccgtggctggcgctggctggtttagttttagcgtttagcgcatcggcg

SEQ ID NO: 241 - protein, 20 aa, synthetic construct
  REGION 1..20: note = Description of Artificial Sequence: Synthetic peptide
MKKITAAAGLLLLAAFSASA

SEQ ID NO: 242 - DNA, 60 bp, synthetic construct
  misc_feature 1..60: note = Description of Artificial Sequence: Synthetic oligonucleotide
  source 1..60: mol_type = other DNA
atgaaaaagattaccgctgctgctggtctgctgctcctcgctgcgtttagcgcatcggcg

SEQ ID NO: 243 - protein, 19 aa, synthetic construct
  REGION 1..19: note = Description of Artificial Sequence: Synthetic peptide
MKKIWLALAGLVLAQPAMA

SEQ ID NO: 244 - DNA, 57 bp, synthetic construct
  misc_feature 1..57: note = Description of Artificial Sequence: Synthetic oligonucleotide
  source 1..57: mol_type = other DNA
atgaaaaagatttggctggcgctggctggtttagttttagcccagccggcgatggcg

SEQ ID NO: 245 - protein, 19 aa, synthetic construct
  REGION 1..19: note = Description of Artificial Sequence: Synthetic peptide
MKKILVLGALALWAQPAMA

SEQ ID NO: 246 - DNA, 57 bp, synthetic construct
  misc_feature 1..57: note = Description of Artificial Sequence: Synthetic oligonucleotide
  source 1..57: mol_type = other DNA
atgaaaaagattttagttttaggtgctctggcgctgtgggcccagccggcgatggcg

SEQ ID NO: 247 - protein, 19 aa, synthetic construct
  REGION 1..19: note = Description of Artificial Sequence: Synthetic peptide
MKKIWLALVLLAGAQPAMA

SEQ ID NO: 248 - DNA, 57 bp, synthetic construct
  misc_feature 1..57: note = Description of Artificial Sequence: Synthetic oligonucleotide
  source 1..57: mol_type = other DNA
atgaaaaagatttggctggcgttagttttactggctggtgcccagccggcgatggcg

SEQ ID NO: 249 - protein, 19 aa, synthetic construct
  REGION 1..19: note = Description of Artificial Sequence: Synthetic peptide
MKKILAGWLALVLAQPAMA

SEQ ID NO: 250 - DNA, 57 bp, synthetic construct
  misc_feature 1..57: note = Description of Artificial Sequence: Synthetic oligonucleotide
  source 1..57: mol_type = other DNA
atgaaaaagattctggctggttggctggcgttagttttagcccagccggcgatggcg

SEQ ID NO: 251 - protein, 19 aa, synthetic construct
  REGION 1..19: note = Description of Artificial Sequence: Synthetic peptide
MKKILVLLAGWLAAQPAMA

SEQ ID NO: 252 - DNA, 57 bp, synthetic construct
  misc_feature 1..57: note = Description of Artificial Sequence: Synthetic oligonucleotide
  source 1..57: mol_type = other DNA
atgaaaaagattttagttttactggctggttggctggcggcccagccggcgatggcg

SEQ ID NO: 253 - protein, 20 aa, synthetic construct
  REGION 1..20: note = Description of Artificial Sequence: Synthetic peptide
MKKITAAAGLLLLAAQPAMA

SEQ ID NO: 254 - DNA, 60 bp, synthetic construct
  misc_feature 1..60: note = Description of Artificial Sequence: Synthetic oligonucleotide
  source 1..60: mol_type = other DNA
atgaaaaagattaccgctgctgctggtctgctgctcctcgctgcccagccggcgatggcg

SEQ ID NO: 255 - protein, 20 aa, synthetic construct
  REGION 1..20: note = Description of Artificial Sequence: Synthetic peptide
MKKILLLLGTAAAAAQPAMA

SEQ ID NO: 256 - DNA, 60 bp, synthetic construct
  misc_feature 1..60: note = Description of Artificial Sequence: Synthetic oligonucleotide
  source 1..60: mol_type = other DNA
atgaaaaagattctgctgctcctcggtaccgctgctgctgctgcccagccggcgatggcg

SEQ ID NO: 257 - protein, 20 aa, synthetic construct
  REGION 1..20: note = Description of Artificial Sequence: Synthetic peptide
MKKILLLLLLLLLLAQPAMA

SEQ ID NO: 258 - DNA, 60 bp, synthetic construct
  misc_feature 1..60: note = Description of Artificial Sequence: Synthetic oligonucleotide
  source 1..60: mol_type = other DNA
atgaaaaagattctgctgctcctcctgctgctcctcctgctcgcccagccggcgatggcg

SEQ ID NO: 259 - protein, 20 aa, synthetic construct
  REGION 1..20: note = Description of Artificial Sequence: Synthetic peptide
MKKIAAAAAAAAAAAQPAMA

SEQ ID NO: 260 - DNA, 60 bp, synthetic construct
  misc_feature 1..60: note = Description of Artificial Sequence: Synthetic oligonucleotide
  source 1..60: mol_type = other DNA
atgaaaaagattgctgctgctgctgcggcggcggcggctgcggcccagccggcgatggcg

SEQ ID NO: 261 - protein, 21 aa, synthetic construct
  REGION 1..21: note = Description of Artificial Sequence: Synthetic peptide
MKYLLPWLALAGLVLAQPAMA

SEQ ID NO: 262 - DNA, 63 bp, synthetic construct
  misc_feature 1..63: note = Description of Artificial Sequence: Synthetic oligonucleotide
  source 1..63: mol_type = other DNA
atgaaatacctgctgccgtggctggcgctggctggtttagttttagcccagccggcgatggcg

SEQ ID NO: 263 - protein, 22 aa, synthetic construct
  REGION 1..22: note = Description of Artificial Sequence: Synthetic peptide
MKYLLPTAAAGLLLLAAFSASA

SEQ ID NO: 264 - DNA, 66 bp, synthetic construct
  misc_feature 1..66: note = Description of Artificial Sequence: Synthetic oligonucleotide
  source 1..66: mol_type = other DNA
atgaaatacctgctgccgaccgctgctgctggtctgctgctcctcgctgcgtttagcgcatcggcg

(19) United States
(12) Patent Application Publication
(10) Pub. No.: US 2024/0218027 A1
(43) Pub. Date: Jul. 4, 2024

(54) NUCLEOSIDE TRIPHOSPHATE TRANSPORTER AND USES THEREOF

(71) Applicant: THE SCRIPPS RESEARCH INSTITUTE, La Jolla, CA (US)

(72) Inventors: Floyd E. ROMESBERG, La Jolla, CA (US); Yorke ZHANG, San Diego, CA (US)

(21) Appl. No.: 18/503,341

(22) Filed: Nov. 7, 2023

Related U.S. Application Data
(63) Continuation of application No. 16/839,741, filed on Apr. 3, 2020, now Pat. No. 11,834,479.
Application No. 16/839,741 is a continuation of application No. 16/546,100, filed on Aug. 20, 2019, now Pat. No. 10,696,720.
Application No. 16/546,100 is a continuation of application No. 16/312,901, filed on Dec. 21, 2018, now abandoned.
(60) Provisional application No. 62/354,650, filed on Jun. 24, 2016.

(51) Int. Cl. C07K 14/405; C12N 9/22; C12N 15/10; C12N 15/11; C12N 15/90

(52) U.S. Cl. CPC C07K 14/405; C12N 9/22; C12N 15/10; C12N 15/11; C12N 15/90; C12N 2310/20; C12N 2800/80

(57) ABSTRACT

Disclosed herein are proteins, methods, cells, engineered microorganisms, and kits for generating a modified nucleoside triphosphate transporter from Phaeodactylum tricornutum. Also disclosed herein proteins, methods, cells, engineered microorganisms, and kits for production of a nucleic acid molecule that comprises an unnatural nucleotide utilizing a modified nucleoside triphosphate transporter from Phaeodactylum tricornutum.

### CROSS-REFERENCE

This application is a continuation of U.S. application Ser. No. 16/839,741 filed Apr. 3, 2020, which is a continuation of U.S. application Ser. No. 16/546,100 filed Aug. 20, 2019 (now U.S. Pat. No. 10,696,720) which is a continuation of U.S. application Ser. No. 16/312,901 filed Dec. 21, 2018, which is a National Stage Entry of PCT/US2017/039133 filed Jun. 23, 2017, which claims the benefit of U.S. Provisional Application No. 62/354,650 filed Jun. 24, 2016. Each of the aforementioned applications are incorporated by reference herein in their entireties, and each is hereby expressly made a part of this specification.

### STATEMENT OF GOVERNMENT SUPPORT

This invention was made with government support under GM060005 awarded by The National Institutes of Health. The government has certain rights in the invention.

### SEQUENCE LISTING

The instant application contains a Sequence Listing which has been submitted electronically in XML format and is hereby incorporated by reference in its entirety. Said XML copy, was created on Jan. 30, 2024, is named “01183-5004-04US—SYN-ST26.xml” and is 392,239 bytes in size.

### BACKGROUND OF THE DISCLOSURE

Oligonucleotides and their applications have revolutionized biotechnology. However, the oligonucleotides including both DNA and RNA each includes only the four natural nucleotides of adenosine (Δ), guanosine (G), cytosine (C), thymine (T) for DNA, and the four natural nucleotides of adenosine (Δ), guanosine (G), cytosine (C), and uridine (U) for RNA, and which significantly restricts the potential functions and applications of the oligonucleotides.

The ability to sequence-specifically synthesize/amplify oligonucleotides (DNA or RNA) with polymerases, for example by PCR or isothermal amplification systems (e.g., transcription with T7 RNA polymerase), has revolutionized biotechnology. In addition to all of the potential applications in nanotechnology, this has enabled a diverse range of new technologies such as the in vitro evolution via SELEX(Systematic Evolution of Ligands by Exponential Enrichment) of RNA and DNA aptamers and enzymes. See, for example, Oliphant AR, Brandl CJ & Struhl K (1989), Defining the sequence specificity of DNA-binding proteins by selecting binding sites from random-sequence oligonucleotides: analysis of yeast GCN4 proteins, Mol. CellBiol., 9:2944-2949; Tuerk C & Gold L (1990), Systematic evolution of ligands by exponential enrichment: RNA ligands to bacteriophage T4 DNA polymerase, Science, 249:505-510; Ellington AD & Szostak JW (1990), In vitro selection of RNA molecules that bind specific ligands, Nature, 346:818-822.

In some aspects, these applications are restricted by the limited chemical/physical diversity present in the natural genetic alphabet (the four natural nucleotides A, C, G, and T in DNA, and the four natural nucleotides A, C, G, and U in RNA).

### SUMMARY OF THE DISCLOSURE

Disclosed herein, in certain embodiments, is an isolated and modified nucleoside triphosphate transporter from Phaeodactylum tricornutum (PtNTT2) comprising a deletion, wherein the isolated and modified nucleoside triphosphate transporter is obtained from an engineered cell. In some embodiments, the deletion is a terminal deletion or an internal deletion. In some embodiments, the deletion is a terminal deletion. In some embodiments, the deletion is an internal deletion. In some embodiments, the terminal deletion is a N-terminal deletion, a C-terminal deletion, or a deletion of both termini. In some embodiments, the terminal deletion is a N-terminal deletion. In some embodiments, the deletion comprises about 5, 10, 15, 20, 22, 25, 30, 40, 44, 50, 60, 66, 70, or more amino acid residues. In some embodiments, the isolated and modified nucleoside triphosphate transporter comprises a deletion of about 5, 10, 15, 20, 22, 25, 30, 40, 44, 50, 60, 66, 70, or more amino acid residues at the N-terminus. In some embodiments, the isolated and modified nucleoside triphosphate transporter comprises a deletion of about 66 amino acid residues at the N-terminus. In some embodiments, the isolated and modified nucleoside triphosphate transporter comprises at least 80%, 85%, 90%, 95%, 96%, 97%, 98%, or 99% sequence identity sequence identity to SEQ ID NO: 4. In some embodiments, the isolated and modified nucleoside triphosphate transporter comprises 100% sequence identity to SEQ ID NO: 4. In some embodiments, the isolated and modified nucleoside triphosphate transporter comprises at least 80%, 85%, 90%, 95%, 96%, 97%, 98%, or 99% sequence identity to SEQ ID NO: 6. In some embodiments, the isolated and modified nucleoside triphosphate transporter comprises 100% sequence identity to SEQ ID NO: 6. In some embodiments, the isolated and modified nucleoside triphosphate transporter comprises at least 80%, 85%, 90%, 95%, 96%, 97%, 98%, or 99% sequence identity to SEQ ID NO: 8. In some embodiments, the isolated and modified nucleoside triphosphate transporter comprises 100% sequence identity to SEQ ID NO: 8. In some embodiments, the engineered cell comprises a prokaryotic cell. In some embodiments, the engineered cell is E. coli.

Disclosed herein, in certain embodiments, is a nucleic acid molecule encoding an isolated and modified nucleoside triphosphate transporter described above.

Disclosed herein, in certain embodiments, is use of a modified nucleoside triphosphate transporter described above for the incorporation of an unnatural triphosphate during the synthesis of a nucleic acid molecule.

Disclosed herein, in certain embodiments, is an engineered cell comprising a first nucleic acid molecule encoding a modified nucleoside triphosphate transporter from Phaeodactylum tricornutum (PtNTT2). In some embodiments, the nucleic acid of the modified nucleoside triphosphate transporter is incorporated in the genomic sequence of the engineered cell. In some embodiments, the engineered cell comprises a plasmid comprising the modified nucleoside triphosphate transporter. In some embodiments, the modified nucleoside triphosphate transporter is a codon optimized nucleoside triphosphate transporter from Phaeodactylum tricornutum. In some embodiments, the modified nucleoside triphosphate transporter comprises a deletion. In some embodiments, the deletion is a terminal deletion or an internal deletion. In some embodiments, the deletion is a N-terminal truncation, a C-terminal truncation, or a truncation of both termini. In some embodiments, the modified nucleoside triphosphate transporter comprises a deletion of about 5, 10, 15, 20, 22, 25, 30, 40, 44, 50, 60, 66, 70, or more amino acid residues. In some embodiments, the modified nucleoside triphosphate transporter comprises a deletion of about 5, 10, 15, 20, 22, 25, 30, 40, 44, 50, 60, 66, 70, or more amino acid residues at the N-terminus. In some embodiments, the modified nucleoside triphosphate transporter comprises a deletion of about 66 amino acid residues at the N-terminus. In some embodiments, the isolated and modified nucleoside triphosphate transporter comprises at least 80%, 85%, 90%, 95%, 96%, 97%, 98%, or 99% sequence identity sequence identity to SEQ ID NO: 4. In some embodiments, the isolated and modified nucleoside triphosphate transporter comprises 100% sequence identity to SEQ ID NO: 4. In some embodiments, the isolated and modified nucleoside triphosphate transporter comprises at least 80%, 85%, 90%, 95%, 96%, 97%, 98%, or 99% sequence identity to SEQ ID NO: 6. In some embodiments, the isolated and modified nucleoside triphosphate transporter comprises 100% sequence identity to SEQ ID NO: 6. In some embodiments, the isolated and modified nucleoside triphosphate transporter comprises at least 80%, 85%, 90%, 95%, 96%, 97%, 98%, or 99% sequence identity to SEQ ID NO: 8. In some embodiments, the isolated and modified nucleoside triphosphate transporter comprises 100% sequence identity to SEQ ID NO: 8. In some embodiments, the modified nucleoside triphosphate transporter is under the control of a promoter selected from an E. coli promoter or a phage promoter. In some embodiments, the promoter is selected from P_{bla}, P_{lac}, P_{lacUV5}, P_{H207}, Pλ, P_{tac}, or P_{N25}. In some embodiments, the modified nucleoside triphosphate transporter is under the control of promoter P_{lacUV5}. In some embodiments, the modified nucleoside triphosphate transporter is under the control of a promoter from a lac operon. In some embodiments, the modified nucleoside triphosphate transporter is encoded within a pSC plasmid. In some embodiments, the modified nucleoside triphosphate transporter decreases doubling time of the engineered cell. In some embodiments, the modified nucleoside triphosphate transporter enables unnatural base pair retention of about 50%, 60%, 70%, 80%, 90%, 95%, 99% or more. In some embodiments, the engineered cell further comprises a second nucleic acid molecule encoding a Cas9 polypeptide or variants thereof, a third nucleic acid molecule encoding a single guide RNA (sgRNA) comprising a crRNA-tracrRNA scaffold; and a fourth nucleic acid molecule comprising an unnatural nucleotide. In some embodiments, the second nucleic acid molecule, the third nucleic acid molecule, and the fourth nucleic acid molecule are encoded in one or more plasmids. In some embodiments, the sgRNA encoded by the third nucleic acid molecule comprises a target motif that recognizes a modification at the unnatural nucleotide position within the fourth nucleic acid molecule. In some embodiments, the modification at the unnatural nucleotide position within the third nucleic acid molecule generates a modified third nucleic acid molecule. In some embodiments, the modification is a substitution. In some embodiments, the modification is a deletion. In some embodiments, the modification is an insertion. In some embodiments, the sgRNA encoded by the third nucleic acid molecule further comprises a protospacer adjacent motif (PAM) recognition element. In some embodiments, the PAM element is adjacent to the 3′ terminus of the target motif. In some embodiments, the combination of Cas9 polypeptide or variants thereof and sgRNA modulates replication of the modified fourth nucleic acid molecule. In some embodiments, the combination of Cas9 polypeptide or variants thereof, sgRNA and the modified nucleoside triphosphate transporter modulates replication of the modified fourth nucleic acid molecule. In some embodiments, the combination of Cas9 polypeptide or variants thereof, sgRNA and the modified nucleoside triphosphate transporter decreases the replication rate of the modified fourth nucleic acid molecule by about 80%, 85%, 95%, 99%, or higher. In some embodiments, the production of the fourth nucleic acid molecule in the engineered cell increases by about 50%, 60%, 70%, 80%, 90%, 95%, 96%, 97%, 98%, 99%, or higher. In some embodiments, the Cas9 polypeptide or variants thereof generate a double-stranded break. In some embodiments, the Cas9 polypeptide is a wild-type Cas9. In some embodiments, the unnatural nucleotide comprises an unnatural base selected from the group consisting of 2-aminoadenin-9-yl, 2-aminoadenine, 2-F-adenine, 2-thiouracil, 2-thio-thymine, 2-thiocytosine, 2-propyl and alkyl derivatives of adenine and guanine, 2-amino-adenine, 2-amino-propyl-adenine, 2-aminopyridine, 2-pyridone, 2′-deoxyuridine, 2-amino-2′-deoxyadenosine 3-deazaguanine, 3-deazaadenine, 4-thio-uracil, 4-thio-thymine, uracil-5-yl, hypoxanthin-9-yl (I), 5-methyl-cytosine, 5-hydroxymethyl cytosine, xanthine, hypoxanthine, 5-bromo, and 5-trifiuoromethyl uracils and cytosines; 5-halouracil, 5-halocytosine, 5-propynyl-uracil, 5-propynyl cytosine, 5-uracil, 5-substituted, 5-halo, 5-substituted pyrimidines, 5-hydroxycytosine, 5-bromocytosine, 5-bromouracil, 5-chlorocytosine, chlorinated cytosine, cyclocytosine, cytosine arabinoside, 5-fluorocytosine, fluoropyrimidine, fluorouracil, 5,6-dihydrocytosine, 5-iodocytosine, hydroxyurea, iodouracil, 5-nitrocytosine, 5-bromouracil, 5-chlorouracil, 5-fluorouracil, and 5-iodouracil, 6-alkyl derivatives of adenine and guanine, 6-azapyrimidines, 6-azo-uracil, 6-azo cytosine, azacytosine, 6-azo-thymine, 6-thio-guanine, 7-methylguanine, 7-methyladenine, 7-deazaguanine, 7-deazaguanosine, 7-deaza-adenine, 7-deaza-8-azaguanine, 8-azaguanine, 8-azaadenine, 8-halo, 8-amino, 8-thiol, 8-thioalkyl, and 8-hydroxyl substituted adenines and guanines; N4-ethylcytosine, N-2 substituted purines, N-6 substituted purines, 0-6 substituted purines, those that increase the stability of duplex formation, universal nucleic acids, hydrophobic nucleic acids, promiscuous nucleic acids, size-expanded nucleic acids, fluorinated nucleic acids, tricyclic pyrimidines, phenoxazine cytidine([5,4-b][1,4]benzoxazin-2(3H)-one), phenothiazine cytidine (1H— pyrimido[5,4-b][1,4]benzothiazin-2(3H)-one), G-clamps, phenoxazine cytidine (9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one), 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methythio-N6-isopentenyladeninje, uracil-5oxyacetic acid, wybutoxosine, pseudouracil, queosine, 2-thiocytosine, 5-methyl-2-thiouracil, 2-thiouracil, 4-thiouracil, 5-methyluracil, uracil-5-oxacetic acid methylester, uracil-5-oxacetic acid, 5-methyl-2-thiouracil, 3-(3-amino-3-N-2-carboxypropyl) uracil, (acp3)w, and 2,6-diaminopurine and those in which the purine or pyrimidine base is replaced with a heterocycle. In some embodiments, the unnatural base is selected from the group consisting of

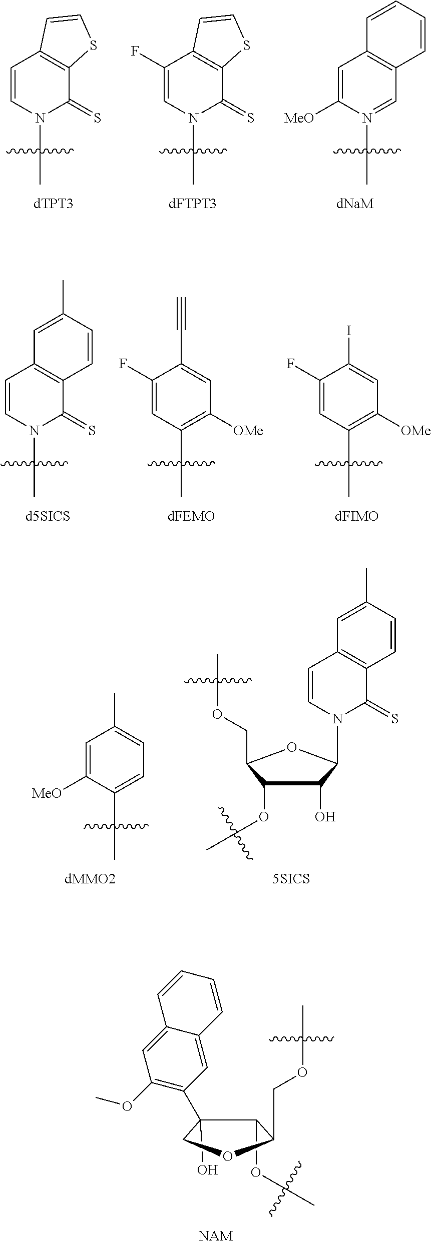

In some embodiments, the unnatural nucleotide further comprises an unnatural sugar moiety. In some embodiments, the unnatural sugar moiety is selected from the group consisting of a modification at the 2′ position: OH; substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃,NH₂F; O-alkyl, S-alkyl, N-alkyl; O-alkenyl, S— alkenyl, N-alkenyl; O-alkynyl, S-alkynyl, N-alkynyl; O-alkyl-O-alkyl, 2′-F, 2′-OCH₃, 2′—O(CH₂)₂OCH₃ wherein the alkyl, alkenyl and alkynyl may be substituted or unsubstituted C₁-C₁₀, alkyl, C₂-C₁₀ alkenyl, C₂-C₁₀ alkynyl, —O[(CH₂)n O]mCH₃, —O(CH₂)nOCH₃, —O(CH₂)n NH₂, —O(CH₂)n CH₃, —O(CH₂)n—ONH₂, and —O(CH₂)nON[(CH₂)n CH₃)]₂, where n and m are from 1 to about 10; and/or a modification at the 5′ position: 5′-vinyl, 5′-methyl (R or S), a modification at the 4′ position, 4′-S, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and any combination thereof. In some embodiments, the unnatural nucleotide further comprises an unnatural backbone. In some embodiments, the unnatural backbone is selected from the group consisting of a phosphorothioate, chiral phosphorothioate, phosphorodithioate, phosphotriester, aminoalkylphosphotriester, C₁-C₁₀ phosphonates, 3′-alkylene phosphonate, chiral phosphonates, phosphinates, phosphoramidates, 3′-amino phosphoramidate, aminoalkylphosphoramidates, thionophosphoramidates, thionoalkylphosphonates, thionoalkylphosphotriesters, and boranophosphates. In some embodiments, the sgRNA has less than about 20%, 15%, 10%, 5%, 3%, 1%, or less off-target binding rate. In some embodiments, the engineered cell further comprises an additional nucleic acid molecule that encodes an additional single guide RNA (sgRNA) comprising a crRNA-tracrRNA scaffold. In some embodiments, the engineered cell is a semi-synthetic organism.

Disclosed herein, in certain embodiments, is an in vivo method of increasing the production of a nucleic acid molecule containing an unnatural nucleotide comprising an engineered cell described above.

Disclosed herein, in certain embodiments, is a nucleic acid molecule containing an unnatural nucleotide produced by an engineered cell described above.

Disclosed herein, in certain embodiments, is an isolated and purified plasmid comprising a nucleic acid molecule encoding a modified nucleoside triphosphate transporter from Phaeodactylum tricornutum (PtNTT2); and a promoter region selected from a pSC plasmid or lacZYA locus. In some embodiments, the modified nucleoside triphosphate transporter is a codon optimized nucleoside triphosphate transporter from Phaeodactylum tricornutum. In some embodiments, the modified nucleoside triphosphate transporter comprises a deletion. In some embodiments, the deletion is a terminal deletion or an internal deletion. In some embodiments, the deletion is a N-terminal truncation, a C-terminal truncation, or a truncation of both termini. In some embodiments, the modified nucleoside triphosphate transporter comprises a deletion of about 5, 10, 15, 20, 22, 25, 30, 40, 44, 50, 60, 66, 70, or more amino acid residues. In some embodiments, the modified nucleoside triphosphate transporter comprises a deletion of about 5, 10, 15, 20, 22, 25, 30, 40, 44, 50, 60, 66, 70, or more amino acid residues at the N-terminus. In some embodiments, the modified nucleoside triphosphate transporter comprises a deletion of about 66 amino acid residues at the N-terminus. In some embodiments, the isolated and modified nucleoside triphosphate transporter comprises at least 80%, 85%, 90%, 95%, 96%, 97%, 98%, or 99% sequence identity sequence identity to SEQ ID NO: 4. In some embodiments, the isolated and modified nucleoside triphosphate transporter comprises 100% sequence identity to SEQ ID NO: 4. In some embodiments, the isolated and modified nucleoside triphosphate transporter comprises at least 80%, 85%, 90%, 95%, 96%, 97%, 98%, or 99% sequence identity to SEQ ID NO: 6. In some embodiments, the isolated and modified nucleoside triphosphate transporter comprises 100% sequence identity to SEQ ID NO: 6. In some embodiments, the isolated and modified nucleoside triphosphate transporter comprises at least 80%, 85%, 90%, 95%, 96%, 97%, 98%, or 99% sequence identity to SEQ ID NO: 8. In some embodiments, the isolated and modified nucleoside triphosphate transporter comprises 100% sequence identity to SEQ ID NO: 8. In some embodiments, the promoter region is selected from P_{bla}, P_{lac}, P_{lacUV5}, P_{H207}, Pλ, P_{tac}, or P_{N25}. In some embodiments, the promoter region is selected from P_{lacI}, P_{bla}, or P_{lac}. In some embodiments, the plasmid is a prokaryotic plasmid.

Disclosed herein, in certain embodiments, is an in vivo method of increasing the production of a nucleic acid molecule containing an unnatural nucleotide comprising incubating a cell with an isolated and purified plasmid described above.

Disclosed herein, in certain embodiments, is a kit comprising an isolated and modified nucleoside triphosphate transporter described above.

Disclosed herein, in certain embodiments, is a kit comprising an engineered cell described above.

Disclosed herein, in certain embodiments, is a kit comprising an isolated and purified plasmid described above.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A-FIG. 1B illustrate UBPs and transporter optimization. FIG. 1A shows the chemical structure of the dNaM-d5SICS and dNaM-dTPT3 UBPs compared to the natural dC-dG base pair. FIG. 1B shows comparison of fitness and [α−³²P]-dATP uptake in DM1 and the various constructed strains: pCDF and inducible PtNTT2(1-575) (gray); pSC and constitutive PtNTT2(66-575) (blue); integrated and constitutive PtNTT2(66-575) (green). Open triangles denote corresponding control strains without PtNTT2. pCDF plasmids are in E. coli C41(DE3); pSC plasmids and integrants are in E. coli BL21(DE3). All PtNTT2 strains are non-codon optimized for plasmid-based expression and codon-optimized for chromosomal expression unless otherwise indicated. r.d.u=relative decay units. Error bars represent s.d. of the mean, n=3 cultures grown and assayed in parallel; the error bars on some data points are smaller than their marker.

FIG. 2A-FIG. 2B illustrate increased UBP retention resulting from transporter and UBP optimization. FIG. 2A shows UBP retentions of plasmids pUCX1, pUCX2, and pBRX2 in strains DM1 and YZ3. Error bars represent s.d. of the mean, n=4 transformations for pUCX1 and pUCX2, n=3 for DM1 pBRX2 and n=5 for YZ3 pBRX2. FIG. 2B shows UBP retentions of pUCX2 variants, wherein the UBP is flanked by all possible combinations of natural nucleotides (NXN, where N=G, C, A, or T and X=NaM), in strain YZ3 grown in media supplemented with either dNaMTP and d5SICSTP (grey bars) or dNaMTP and dTPT3TP (black bars).

FIG. 3A-FIG. 3C illustrate the Cas9-based editing system. FIG. 3A illustrates the model for Cas9-mediated immunity to UBP loss. FIG. 3B shows UBP retentions of pUCX2 variants in strain YZ2 with a pCas9 plasmid that expresses a non-target sgRNA (gray) or an on-target sgRNA (black). Error bars represent s.d. of the mean, n=3 transformations for all sequences except on-target CXA and CXG, where n=5. FIG. 3C shows UBP retentions of pAIO plasmids in strain YZ3 (gray), which does not express Cas9, or in strain YZ4 (black) with expression of Cas9. In FIG. 3B and FIG. 3C, the nucleotides immediately flanking X=NaM are indicated, as is distance to the PAM. (N) denotes the nucleotide N in the sgRNA that targets a substitution mutation of the UBP; all pCas9 and pAIO plasmids also express an sgRNA targeting the deletion mutation. Error bars represent s.d. of the mean, n≥3 colonies; see FIG. 11 for exact values of n, sequences, and IPTG concentrations used to induced Cas9 in YZ4.

FIG. 4 shows simultaneous retention of two UBPs during extended growth. Strains YZ3 and YZ4 were transformed with pAIO2X and plated on solid media containing dNaMTP and dTPT3TP, with or without IPTG to induce Cas9. Single colonies were inoculated into liquid media of the same composition and cultures were grown to an OD₆₀₀ of ˜2 (point 1). Cultures were subsequently diluted 30,000-fold and regrown to an OD₆₀₀ of ˜2 (point 2), and this dilution-regrowth process was then repeated two more times (points 3 and 4). As a no immunity control, strain YZ3 was grown in the absence of IPTG and two representative cultures are indicated in gray. Strain YZ4 was grown in the presence of varying amounts of IPTG and averages of cultures are indicated in green (0 μM, n=5), blue (20 μM, n=5), and red (40 μM, n=4). Retentions of the UBP in gfp and serT are indicated with solid or dotted lines, respectively. After the fourth outgrowth, two of the YZ4 cultures grown with 20 μM IPTG were subcultured on solid media of the same composition. Three randomly selected colonies from each plate (n=6 total) were inoculated into liquid media of the same composition, and each of the six cultures was grown to an OD₆₀₀ of ˜1 (point 5), diluted 300,000-fold into media containing 0, 20, and 40 μM IPTG, and regrown to an OD₆₀₀ of ˜1 (point 6). This dilution-regrowth process was subsequently repeated (point 7). pAIO2X plasmids were isolated at each of the numbered points and analyzed for UBP retention. Cell doublings are estimated from OD₆₀₀ (see Methods) and did not account for growth on solid media (thus making them an underestimate of actual growth). Error bars represent s.d. of the mean.

FIG. 5A-FIG. 5F illustrate dATP uptake and growth of cells expressing PtNTT2 as a function of inducer (IPTG) concentration or promoter strength, strain background and presence of N-terminal signal sequences. FIG. 5A and FIG. 5D show uptake of [α−³²P]-dATP. Error bars represent s.d. of the mean, n=3 cultures. r.d.u.=relative decay units, which corresponds to the total number of radioactive counts per minute normalized to the average OD₆₀₀ across the 1 h window of uptake, with the uptake of C41(DE3) pCDF-1b PtNTT2(1-575) (i.e. DM1) induced with 1000 μM IPTG set to 1. Deletion of the N-terminal signal sequences drastically reduces uptake activity in C41(DE3), but activity can be restored with higher levels of expression in BL21(DE3). FIG. 5B shows growth curves of C41(DE3) strains. Induction of PtNTT2(1-575) is toxic. FIG. 5C shows growth curves of BL21(DE3) strains. Induction of T7 RNAP in BL21(DE3) is toxic (see empty vector traces), which masks the effect of deleting the N-terminal signal sequences of PtNTT2 on cell growth. FIG. 5E shows growth curves of plasmid-based transporter strains. Strains are expressing non codon-optimized (co) PtNTT2(66-575) unless otherwise indicated. FIG. 5F shows growth curves of chromosomally-integrated transporter strains. Strains are expressing codon-optimized PtNTT2(66-575) unless otherwise indicated. Strain YZ4 also contains a chromosomally integrated Cas9 gene.

FIG. 6 shows plasmid maps. Promoters and terminators are denoted by white and gray features, respectively. * denotes the derivative of the pMB1 origin from pUC19, which contains a mutation that increases its copy number. Plasmids that contain a UBP are generally indicated with the TK1 sequence (orange), but as described in the text and indicated above, pUCX2 and pAIO variants with other UBP-containing sequences also position the UBP in the approximate locus shown with TK1 above. sgRNA (N) denotes the guide RNA that recognizes a natural substitution mutation of the UBP, with N being the nucleotide present in the guide RNA. sgRNA (Δ) denotes the guide RNA that recognizes a single base pair deletion of the UBP; this and its associated promoter and terminator (indicated by ‡) are only present in certain experiments. The serT and gfp genes do not have promoters.

FIG. 7A-FIG. 7B illustrate biotin shift assay gels. FIG. 7A shows biotin shift assay scheme and representative gels for FIG. 2b. Input plasmid refers to the ligation product used to transform the SSO. * denotes a band whose mobility does not change in the absence of streptavidin (data not shown) and does not appear in any samples from clonally-derived cultures (data not shown). The band likely corresponds to a fully natural plasmid derived from non-specific priming during the PCR used to generate the insert for ligation, and is present in very small quantities in the input plasmid, but is enriched for during replication in vivo by competition against challenging UBP sequences. Such bands are not included in the calculation of retention. FIG. 7B illustrates representative gels for FIG. 4. Each lane (excluding the oligonucleotide controls) corresponds to a pAIO2X plasmid sample isolated from a clonally-derived YZ4 culture, grown with the IPTG concentration indicated, after an estimated 108 cell doublings in liquid culture (point 7 in FIG. 4). Each plasmid sample is split and analyzed in parallel biotin shift reactions that assay the UBP content at the gfp and serT loci (red and blue primers, respectively). The 80 μM samples are not shown in the plot for FIG. 4.

FIG. 8A-FIG. 8B show additional characterization of UBP propagation. FIG. 8A shows growth curves for the experiments shown in FIG. 2a. YZ3 and DM1 (induced with 1 mM IPTG) were transformed with the indicated UBP-containing plasmids, or their corresponding fully natural controls, and grown in media containing dNaMTP and d5SICSTP. Each line represents one transformation and subsequent growth in liquid culture. The x-axis represents time spent in liquid culture, excluding the 1 h of recovery following electroporation (see Methods). Growth curves terminate at the OD₆₀₀ at which cells were collected for plasmid isolation and analysis of UBP retention. Staggering of the curves along the x-axis for replicates within a given strain and plasmid combination is likely due to minor variability in transformation frequencies between transformations (and thus differences in the number of cells inoculated into each culture), whereas differences in slope between curves indicate differences in fitness. Growth of YZ3 is comparable between all three UBP-containing plasmids (and between each UBP-containing plasmid with its respective natural control), whereas growth of DM1 is impaired by the UBP-containing plasmids, especially for pUCX1 and pUCX2. FIG. 8B shows retentions of gfp pUCX2 variants propagated in YZ3 by transformation, plating on solid media, isolation of single colonies, and subsequent inoculation and growth in liquid media, in comparison to retentions from plasmids propagated by transformation and growth of YZ3 in liquid media only. Cells were plated from the same transformations described in FIG. 2b. Solid and liquid media both contained dNaMTP and dTPT3TP. Cells were harvested at OD₆₀₀ ˜ 1. Five colonies were inoculated for each of the pUCX2 variants indicated, but some colonies failed to grow (indicated by a blank space in the table). Retentions for samples isolated from transformants grown solely in liquid media were assayed from the same samples shown in FIG. 2b, but were assayed and normalized to an oligonucleotide control in parallel with the plated transformant samples to facilitate comparisons in retention. See Methods for additional details regarding UBP retention normalization. For samples with near zero shift, we cannot determine whether the UBP was completely lost in vivo or if the sample came from a colony that was transformed with a fully natural plasmid (some of which arises during plasmid construction, specifically during the PCR used to generate the UBP-containing insert).

FIG. 9A-FIG. 9B illustrate effect of dNaM-dTPT3 on Cas9-mediated cleavage of DNA in vitro. Cas9-mediated in vitro cleavage was assessed for six DNA substrates, wherein the third base pair upstream of the PAM was either one of the four natural base pairs or the UBP (in both strand contexts). The four sgRNAs that are complementary to each natural template were prepared by in vitro transcription with T7 RNAP. To account for differences in sgRNA activity and/or minor variations in preparation, a relative percent maximal cleavage for each sgRNA vs all six DNA substrates is shown in parentheses. Values represent means±1 s.d. (n=3 technical replicates). In several cases, the presence of an unnatural nucleotide significantly reduced cleavage compared to DNA complementary to the sgRNA. This data suggests that Cas9 programmed with sgRNA(s) complementary to one or more of the natural sequences would preferentially degrade DNA that had lost the UBP. FIG. 9B discloses SEQ ID NOS 201 and 202, respectively, in order of appearance.

FIG. 10A-FIG. 10D show Cas9-mediated immunity to UBP loss in TK1. FIG. 10A shows sgRNA sequences used to enhance retention of the UBP (SEQ ID NOS 203, 204, 204, 205, and 206, respectively, in order of appearance). FIG. 10B shows UBP retention for pUCX2 TK1 is enhanced by targeting Cas9 to the major mutation (dTPT3→dA). As cells continue to grow in the absence of correct sgRNAs targeting mutations, UBP retention declines. Error bars represent s.d. of the mean, n=3 transformations. In FIG. 10A and FIG. 10B, hEGFP is a non-target sgRNA. FIG. 10C shows Sanger sequencing chromatogram illustrating mutation of dNaM to dT in the absence of an sgRNA to target Cas9 nuclease activity (SEQ ID NOS 207 and 208, respectively, in order of appearance). FIG. 10D shows Sanger sequencing chromatogram illustrating that loss of retention in the presence of Cas9 and a targeting sgRNA (TK1-A) is due to growth of cells with plasmids possessing a single base pair mutation. UBP-containing species were depleted before sequencing. FIG. 10D discloses SEQ ID NOS 207 and 208, respectively, in order of appearance.

FIG. 11 shows Cas9 NXN sequences. 22-nt of each UBP-containing sequence examined in FIG. 3 is shown above. X=dNaM, Y=dTPT3 (SEQ ID NOS 209-224, respectively, in order of appearance). The sequence of the sgRNA targeting the substitution mutation of the UBP (N) is the 18-nt sequence 5′ to the NGG PAM with X or Y replaced by the natural nucleotide indicated. The sequence of the sgRNA targeting the deletion mutation of the UBP (Δ) is the 19-nt sequence 5′ to the NGG PAM without X or Y. YZ3 experiments were performed without IPTG. Retentions shown in FIG. 3C are averaged from the values and number of colonies indicated here.

FIG. 12 shows growth curves of YZ4 replicating pAIO2X. Growth curves for the first dilution-regrowth (point 2) in FIG. 4. Curves terminate at the OD₆₀₀ at which cultures were collected for both plasmid isolation and dilution for the next regrowth. Doubling times are calculated from the timepoints collected between OD₆₀₀ 0.1-1.0 for each curve and averaged for each strain and/or IPTG condition.

### DETAILED DESCRIPTION OF THE DISCLOSURE

Nucleosides are hydrophilic molecules which requires transport proteins for permeation of cell membranes. Nucleoside transporters (NTs) are a group of membrane transport proteins that facilitate crossing of the nucleosides through cell membranes and vesicles. In some cases, there are two types of nucleoside transporters, concentrative nucleoside transporters which drives a concentrative process by electrochemical gradient, and equilibrative nucleoside transporters which drives an equilibrative bidirectional process by chemical gradient. In some instances, a nucleoside transporter further encompasses a nucleoside triphosphate transporter.

Natural nucleosides comprise adenine, guanine, thymine, uracil, and cytosine; and are recognized by nucleotide transporters for permeation of cell membranes. Unnatural nucleosides, in some cases, are either not recognized by endogenous nucleotide transporters or are recognized but the efficiency of transport is low.

In some embodiments, described herein are modified nucleotide transporters that recognize and facilitate transport of unnatural nucleic acids into a cell. In some instances, the modified nucleotide transporter enhances import of unnatural nucleic acids into a cell relative to an endogeneous nucleotide transporter. In some cases, the modified nucleotide transporter increases unnatural nucleic acid retention within a cell. In additional cases, the modified nucleotide transporter minimizes toxicity due to its expression, and optionally improves cell doubling time and fitness relative to a cell in the absence of the transporter.

### Nucleoside Triphosphate Transporters

In certain embodiments, described herein are modified nucleoside triphosphate transporters for transporting unnatural nucleic acids into a cell. In some instances, the modified nucleoside triphosphate transporter is from Phaeodactylum tricornutum(PtNTT2). In some instances, the modified nucleoside triphosphate transporter further comprises a deletion. In some cases, the deletion is a terminal deletion (e.g., a N-terminal deletion or a C-terminal deletion) or is an internal deletion.

In some embodiments, described herein is an isolated and modified modified nucleoside triphosphate transporter from Phaeodactylum tricornutum (PtNTT2) comprising a deletion. In some instances, the deletion comprises about 5, 10, 15, 20, 22, 25, 30, 40, 44, 50, 60, 66, 70, 80, 90, or more amino acid residues. In some instances, the deletion comprises about 5, 10, 15, 20, 22, 25, 30, 40, 44, 50, 60, 66, 70, or more amino acid residues. In some cases, the modified modified nucleoside triphosphate transporter from Phaeodactylum tricornutum (PtNTT2) comprises a deletion of about 5 or more amino acid residues. In some cases, the modified modified nucleoside triphosphate transporter from Phaeodactylum tricornutum (PtNTT2) comprises a deletion of about 10 or more amino acid residues. In some cases, the modified modified nucleoside triphosphate transporter from Phaeodactylum tricornutum (PtNTT2) comprises a deletion of about 15 or more amino acid residues. In some cases, the modified modified nucleoside triphosphate transporter from Phaeodactylum tricornutum (PtNTT2) comprises a deletion of about 20 or more amino acid residues. In some cases, the modified modified nucleoside triphosphate transporter from Phaeodactylum tricornutum (PtNTT2) comprises a deletion of about 22 or more amino acid residues. In some cases, the modified modified nucleoside triphosphate transporter from Phaeodactylum tricornutum (PtNTT2) comprises a deletion of about 25 or more amino acid residues. In some cases, the modified modified nucleoside triphosphate transporter from Phaeodactylum tricornutum (PtNTT2) comprises a deletion of about 30 or more amino acid residues. In some cases, the modified modified nucleoside triphosphate transporter from Phaeodactylum tricornutum (PtNTT2) comprises a deletion of about 40 or more amino acid residues. In some cases, the modified modified nucleoside triphosphate transporter from Phaeodactylum tricornutum (PtNTT2) comprises a deletion of about 44 or more amino acid residues. In some cases, the modified modified nucleoside triphosphate transporter from Phaeodactylum tricornutum (PtNTT2) comprises a deletion of about 50 or more amino acid residues. In some cases, the modified modified nucleoside triphosphate transporter from Phaeodactylum tricornutum (PtNTT2) comprises a deletion of about 60 or more amino acid residues. In some cases, the modified modified nucleoside triphosphate transporter from Phaeodactylum tricornutum (PtNTT2) comprises a deletion of about 66 or more amino acid residues. In some cases, the modified modified nucleoside triphosphate transporter from Phaeodactylum tricornutum (PtNTT2) comprises a deletion of about 70 or more amino acid residues.

In some embodiments, described herein is an isolated and modified modified nucleoside triphosphate transporter from Phaeodactylum tricornutum (PtNTT2) comprising a N-terminal deletion. In some instances, the N-terminal deletion comprises about 5, 10, 15, 20, 22, 25, 30, 40, 44, 50, 60, 66, 70, 80, 90, or more amino acid residues. In some instances, the N-terminal deletion comprises about 5, 10, 15, 20, 22, 25, 30, 40, 44, 50, 60, 66, 70, or more amino acid residues. In some cases, the modified modified nucleoside triphosphate transporter from Phaeodactylum tricornutum (PtNTT2) comprises a N-terminal deletion of about 5 or more amino acid residues. In some cases, the modified modified nucleoside triphosphate transporter from Phaeodactylum tricornutum (PtNTT2) comprises a N-terminal deletion of about 10 or more amino acid residues. In some cases, the modified modified nucleoside triphosphate transporter from Phaeodactylum tricornutum (PtNTT2) comprises a N-terminal deletion of about 15 or more amino acid residues. In some cases, the modified modified nucleoside triphosphate transporter from Phaeodactylum tricornutum (PtNTT2) comprises a N-terminal deletion of about 20 or more amino acid residues. In some cases, the modified modified nucleoside triphosphate transporter from Phaeodactylum tricornutum (PtNTT2) comprises a N-terminal deletion of about 22 or more amino acid residues. In some cases, the isolated and modified modified nucleoside triphosphate transporter from Phaeodactylum tricornutum (PtNTT2) comprises a N-terminal deletion of about 25 or more amino acid residues. In some cases, the modified modified nucleoside triphosphate transporter from Phaeodactylum tricornutum (PtNTT2) comprises a N-terminal deletion of about 30 or more amino acid residues. In some cases, the modified modified nucleoside triphosphate transporter from Phaeodactylum tricornutum (PtNTT2) comprises a N-terminal deletion of about 40 or more amino acid residues. In some cases, the modified modified nucleoside triphosphate transporter from Phaeodactylum tricornutum (PtNTT2) comprises a N-terminal deletion of about 44 or more amino acid residues. In some cases, the modified modified nucleoside triphosphate transporter from Phaeodactylum tricornutum (PtNTT2) comprises a N-terminal deletion of about 50 or more amino acid residues. In some cases, the modified modified nucleoside triphosphate transporter from Phaeodactylum tricornutum (PtNTT2) comprises a N-terminal deletion of about 60 or more amino acid residues. In some cases, the modified modified nucleoside triphosphate transporter from Phaeodactylum tricornutum (PtNTT2) comprises a N-terminal deletion of about 66 or more amino acid residues. In some cases, the modified modified nucleoside triphosphate transporter from Phaeodactylum tricornutum (PtNTT2) comprises a N-terminal deletion of about 70 or more amino acid residues.

In some embodiments, the isolated and modified nucleoside triphosphate transporter comprises at least 80%, 85%, 90%, 95%, 96%, 97%, 98%, or 99% sequence identity to SEQ ID NO: 4. In some instances, the modified nucleoside triphosphate transporter comprises at least 80% sequence identity to SEQ ID NO: 4. In some instances, the modified nucleoside triphosphate transporter comprises at least 85% sequence identity to SEQ ID NO: 4. In some instances, the modified nucleoside triphosphate transporter comprises at least 90% sequence identity to SEQ ID NO: 4. In some instances, the modified nucleoside triphosphate transporter comprises at least 95% sequence identity to SEQ ID NO: 4. In some instances, the modified nucleoside triphosphate transporter comprises at least 96% sequence identity to SEQ ID NO: 4. In some instances, the modified nucleoside triphosphate transporter comprises at least 97% sequence identity to SEQ ID NO: 4. In some instances, the modified nucleoside triphosphate transporter comprises at least 98% sequence identity to SEQ ID NO: 4. In some instances, the modified nucleoside triphosphate transporter comprises at least 99% sequence identity to SEQ ID NO: 4. In some instances, the modified nucleoside triphosphate transporter comprises 100% sequence identity to SEQ ID NO: 4. In some instances, the modified nucleoside triphosphate transporter consists of 100% sequence identity to SEQ ID NO: 4.

In some embodiments, the isolated and modified nucleoside triphosphate transporter comprises at least 80%, 85%, 90%, 95%, 96%, 97%, 98%, or 99% sequence identity to SEQ ID NO: 6. In some instances, the modified nucleoside triphosphate transporter comprises at least 80% sequence identity to SEQ ID NO: 6. In some instances, the modified nucleoside triphosphate transporter comprises at least 85% sequence identity to SEQ ID NO: 6. In some instances, the modified nucleoside triphosphate transporter comprises at least 90% sequence identity to SEQ ID NO: 6. In some instances, the modified nucleoside triphosphate transporter comprises at least 95% sequence identity to SEQ ID NO: 6. In some instances, the modified nucleoside triphosphate transporter comprises at least 96% sequence identity to SEQ ID NO: 6. In some instances, the modified nucleoside triphosphate transporter comprises at least 97% sequence identity to SEQ ID NO: 6. In some instances, the modified nucleoside triphosphate transporter comprises at least 98% sequence identity to SEQ ID NO: 6. In some instances, the modified nucleoside triphosphate transporter comprises at least 99% sequence identity to SEQ ID NO: 6. In some instances, the modified nucleoside triphosphate transporter comprises 100% sequence identity to SEQ ID NO: 6. In some instances, the modified nucleoside triphosphate transporter consists of 100% sequence identity to SEQ ID NO: 6.

In some embodiments, the isolated and modified nucleoside triphosphate transporter comprises at least 80%, 85%, 90%, 95%, 96%, 97%, 98%, or 99% sequence identity to SEQ ID NO: 8. In some instances, the modified nucleoside triphosphate transporter comprises at least 80% sequence identity to SEQ ID NO: 8. In some instances, the modified nucleoside triphosphate transporter comprises at least 85% sequence identity to SEQ ID NO: 8. In some instances, the modified nucleoside triphosphate transporter comprises at least 90% sequence identity to SEQ ID NO: 8. In some instances, the modified nucleoside triphosphate transporter comprises at least 95% sequence identity to SEQ ID NO: 8. In some instances, the modified nucleoside triphosphate transporter comprises at least 96% sequence identity to SEQ ID NO: 8. In some instances, the modified nucleoside triphosphate transporter comprises at least 97% sequence identity to SEQ ID NO: 8. In some instances, the modified nucleoside triphosphate transporter comprises at least 98% sequence identity to SEQ ID NO: 8. In some instances, the modified nucleoside triphosphate transporter comprises at least 99% sequence identity to SEQ ID NO: 8. In some instances, the modified nucleoside triphosphate transporter comprises 100% sequence identity to SEQ ID NO: 8. In some instances, the modified nucleoside triphosphate transporter consists of 100% sequence identity to SEQ ID NO: 8.

In some embodiments, a modified nucleoside triphosphate transporter described herein has a specificity for an unnatural nucleic acid that is at least about 10%, 20%, 30%, 40%, 50%, 60%, 70%, 80%, 90%, 95%, 97%, 98%, 99%, 99.5%, 99.99% the specificity of the wild type nucleoside triphosphate transporter toward the unnatural nucleic acid. In some embodiments, the modified nucleoside triphosphate transporter has a specificity for an unnatural nucleic acid comprising a modified sugar that is at least about 10%, 20%, 30%, 40%, 50%, 60%, 70%, 80%, 90%, 95%, 97%, 98%, 99%, 99.5%, 99.99% the specificity of the wild type nucleoside triphosphate transporter toward a natural nucleic acid and/or the unnatural nucleic acid without the modified sugar. In some embodiments, the modified nucleoside triphosphate transporter has a specificity for an unnatural nucleic acid comprising a modified base that is at least about 10%, 20%, 30%, 40%, 50%, 60%, 70%, 80%, 90%, 95%, 97%, 98%, 99%, 99.5%, 99.99% the specificity of the wild type nucleoside triphosphate transporter toward a natural nucleic acid and/or the unnatural nucleic acid without the modified base. In some embodiments, the modified nucleoside triphosphate transporter has a specificity for an unnatural nucleic acid comprising a triphosphate that is at least about 10%, 20%, 30%, 40%, 50%, 60%, 70%, 80%, 90%, 95%, 97%, 98%, 99%, 99.5%, 99.99% the specificity of the wild type nucleoside triphosphate transporter toward a nucleic acid comprising a triphosphate and/or the unnatural nucleic acid without the triphosphate. For example, a modified nucleoside triphosphate transporter can have a specificity for an unnatural nucleic acid comprising a triphosphate that is at least about 10%, 20%, 30%, 40%, 50%, 60%, 70%, 80%, 90%, 95%, 97%, 98%, 99%, 99.5%, 99.99% the specificity of the wild type nucleoside triphosphate transporter toward the unnatural nucleic acid with a diphosphate or monophosphate, or no phosphate, or a combination thereof.

In some embodiments, a modified nucleoside triphosphate transporter described herein has a specificity for an unnatural nucleic acid and a specificity to a natural nucleic acid that is at least about 10%, 20%, 30%, 40%, 50%, 60%, 70%, 80%, 90%, 95%, 97%, 98%, 99%, 99.5%, 99.99% the specificity of the wild type nucleoside triphosphate transporter toward the natural nucleic acid. In some embodiments, the modified nucleoside triphosphate transporter has a specificity for an unnatural nucleic acid comprising a modified sugar and a specificity to a natural nucleic acid that is at least about 10%, 20%, 30%, 40%, 50%, 60%, 70%, 80%, 90%, 95%, 97%, 98%, 99%, 99.5%, 99.99% the specificity of the wild type nucleoside triphosphate transporter toward the natural nucleic acid. In some embodiments, the modified nucleoside triphosphate transporter has a specificity for an unnatural nucleic acid comprising a modified base and a specificity to a natural nucleic acid that is at least about 10%, 20%, 30%, 40%, 50%, 60%, 70%, 80%, 90%, 95%, 97%, 98%, 99%, 99.5%, 99.99% the specificity of the wild type nucleoside triphosphate transporter toward the natural nucleic acid.

In some embodiments, a sequence of a modified nucleoside triphosphate transporter is further modified to improve the expression and cellular activity. In some instances, the codon usage is modified to introduce ribosomal pause sites to slow translation and to improve the targeting of the modified nucleoside triphosphate transporter polypeptide to membrane translocons (Fluman, et al., “mRNA-programmed translation pauses in the targeting of E. coli membrane proteins,” eLife 2014; 3:e03440). In some instances, modification of one or more transmembrane helices, for example, modification of a first transmembrane helix and/or generating a chimeric transporter comprising a first transmembrane helix of a different protein (e.g., a related transporter) may enhance expression and cellular activities (Marshall, et al., “A link between integral membrane protein expression and simulated integration efficiency,” Cell Reports, 16(8): 2169-2177 (2016)). In some instances, an endogenous, a modified, or a heterologous signal peptide is incorporated into the sequence of a modified nucleoside triphosphate transporter to improve expression and cellular activity. In some cases, the signal peptide is optionally linked in-frame with the sequence of the modified nucleoside triphosphate transporter through a linker. In some cases, the linker is a non-cleavable linker. In other cases, the linker is a cleavable linker. Exemplary signal peptides are illustrated in Table 3. In some cases, a signal peptide from Table 3, optionally linked to a linker, is incorporated into the sequence of a modified nucleoside triphosphate transporter described herein.

In some embodiments, the expression of the modified nucleoside triphosphate transporter is tuned through modification of the ribosomal binding site to modulate the rate of the modified nucleoside triphosphate transporter polypeptide's synthesis. See, e.g., Howard, et al., “Automated design of synthetic ribosome binding sites to control protein expression,” Nature Biotechnology 27: 946-950 (2009); and Mutalik, et al., “Precise and reliable gene expression via standard transcription and translation initiation elements,” Nature Methods 10: 354-360 (2013).

In some embodiments, the expression of the modified nucleoside triphosphate transporter is modulated by the attachment of a tunable degradation tag. In some instances, a tunable degradation tag comprises a small amino acid sequence that, when fused to a target protein, marks the protein for degradation by a cognate protease in a bacterial cell. Exemplary tunable degradation tag and cognate protease pairs include, but are not limited to, E. coli ssrA (ec-ssrA)/E. coli Lon (ec-Lon), and Mesoplasmaflorum ssrA (mf-ssrA)/Mesoplasmna florum Lon (mf-Lon). In some instances, the tunable degradation tag comprises a modified tag that alters expression and/or degradation dynamis relative to an unmodified degradation tag. In some instances, a tunable degradation tag contemplated herein comprises a degradation tag described in PCT Patent Publication WO2014/160025A2. In some instances, a tunable degradation tag contemplated herein comprises a degradation tag described in Cameron, et al., “Tunable protein degradation in bacteria,” Nature Biotechnology 32: 1276-1281 (2014).

In some embodiments, the expression of the modified nucleoside triphosphate transporter is modulated by the availability of an endogenous or exogenous (e.g. unnatural nucleotide triphosphate or unnatural amino acid) molecule during translation. In some instances, the expression of the modified nucleoside triphosphate transporter is correlated with the copy number of rare codons, in which the rate of a ribosomal read-through of a rare codon modulates translation of the transporter. See, e.g., Wang, et al., “An engineered rare codon device for optimization of metabolic pathways,” Scientific Reports 6:20608 (2016).

In some instances, a modified nucleoside triphosphate transporter is characterized according to its rate of dissociation from a nucleic acid substrate. In some embodiments, a modified nucleoside triphosphate transporter has a relatively low dissociation rate for one or more natural and unnatural nucleic acids. In some embodiments, a modified nucleoside triphosphate transporter has a relatively high dissociation rate for one or more natural and unnatural nucleic acids. The dissociation rate is an activity of an isolated and modified nucleoside triphosphate transporter that can be adjusted to tune reaction rates in methods set forth herein.

Modified nucleoside triphosphate transporters from native sources or variants thereof can be screened using an assay that detects importation of an unnatural nucleic acid having a particular structure. In one example, the modified nucleoside triphosphate transporters can be screened for the ability to import an unnatural nucleic acid or UBP; e.g., d5SICSTP, dNaMTP, or d5SICSTP-dNaMTP UBP. A NTT, e.g., a heterologous transporter, can be used that displays a modified property for the unnatural nucleic acid as compared to the wild-type transporter. For example, the modified property can be, e.g., Kₘ, k_{cat}, Vₘₐₓ, NTT importation in the presence of an unnatural nucleic acid (or of a naturally occurring nucleotide), average template read-length by a cell with the modified nucleoside triphosphate transporter in the presence of an unnatural nucleic acid, specificity of the transporter for an unnatural nucleic acid, rate of binding of an unnatural nucleic acid, or rate of product release, or any combination thereof. In one embodiment, the modified property is a reduced Kₘ for an unnatural nucleic acid and/or an increased k_{cat}/Kₘ or Vₘₐₓ/Kₘ for an unnatural nucleic acid. Similarly, the modified nucleoside triphosphate transporter optionally has an increased rate of binding of an unnatural nucleic acid, an increased rate of product release, and/or an increased cell importation rate, as compared to a wild-type transporter.

At the same time, a modified nucleoside triphosphate transporter can import natural nucleic acids, e.g., A, C, G, and T, into cell. For example, a modified nucleoside triphosphate transporter optionally displays a specific importation activity for a natural nucleic acid that is at least about 5% as high (e.g., 5%, 10%, 25%, 50%, 75%, 100% or higher), as a corresponding wild-type transporter. Optionally, the modified nucleoside triphosphate transporter displays a k_{cat}/Kₘ or Vₘₐₓ/Kₘ for a naturally occurring nucleotide that is at least about 5% as high (e.g., about 5%, 10%, 25%, 50%, 75% or 100% or higher) as the wild-type NTT.

Modified nucleoside triphosphate transporters used herein that can have the ability to import an unnatural nucleic acid of a particular structure can also be produced using a directed evolution approach. A nucleic acid synthesis assay can be used to screen for transporter variants having specificity for any of a variety of unnatural nucleic acids. For example, transporter variants can be screened for the ability to import an unnatural nucleic acid or UBP; e.g., d5SICSTP, dNaMTP, or d5SICSTP-dNaMTP UBP into nucleic acids. In some embodiments, such an assay is an in vitro assay, e.g., using a recombinant transporter variant. In some embodiments, such an assay is an in vivo assay, e.g., expressing a transporter variant in a cell. Such directed evolution techniques can be used to screen variants of any suitable transporter for activity toward any of the unnatural nucleic acids set forth herein.

### Engineered Cells

In some embodiments, described herein is an engineered cell comprising a nucleic acid molecule encoding a modified nucleoside triphosphate transporter. In some instances, the nucleic acid molecule encodes a modified nucleoside triphosphate transporter from Phaeodactylum tricornutum (PtNTT2). In some instances, the nucleic acid of the modified nucleoside triphosphate transporter is incorporated in the genomic sequence of the engineered cell.

The engineered cell can be any suitable prokaryote. In some instances, the engineered cell is a Gram negative bacteria. In other instances, the engineered cell is a Gram positive bacteria. Exemplary bacteria include, but are not limited to, Bacillus bacteria (e.g., B. subtilis, B. megaterium), Acinetobacter bacteria, Norcardia baceteria, Xanthobacter bacteria, Escherichia bacteria (e.g., E. coli (e.g., strains DH10B, Stbl2, DH5-alpha, DB3, DB3.1), DB4, DB5, JDP682 and ccdA-over (e.g., U.S. application Ser. No. 09/518,188))), Streptomyces bacteria, Erwinia bacteria, Klebsiella bacteria, Serratia bacteria (e.g., S. marcessans), Pseudomonas bacteria (e.g., P. aeruginosa), Salmonella bacteria (e.g., S. typhimurium, S. typhi), Megasphaera bacteria (e.g., Megasphaera elsdenii). Bacteria also include, but are not limited to, photosynthetic bacteria (e.g., green non-sulfur bacteria (e.g., Choroflexus bacteria (e.g., C. aurantiacus), Chloronema bacteria (e.g., C. gigateum)), green sulfur bacteria (e.g., Chlorobium bacteria (e.g., C. limicola), Pelodictyon bacteria (e.g.,P. luteolum), purple sulfur bacteria (e.g., Chromatium bacteria (e.g., C. okenii)), and purple non-sulfur bacteria (e.g., Rhodospirillum bacteria (e.g., R. rubrum), Rhodobacter bacteria (e.g., R. sphaeroides, R. capsulatus), and Rhodomicrobium bacteria (e.g., R. vanellii)).

In some instances, the engineered cell comprises a plasmid comprising the modified nucleoside triphosphate transporter. In some cases, the modified nucleoside triphosphate transporter is a codon optimized nucleoside triphosphate transporter from Phaeodactylum tricornutum.

In some embodiments, the modified nucleoside triphosphate transporter comprises a deletion. In some cases, the deletion is a terminal deletion (e.g., a N-terminal or a C-terminal deletion). In other cases, the deletion is an internal deletion.

As described above, the modified nucleoside triphosphate transporter comprises a deletion of about 5, 10, 15, 20, 22, 25, 30, 40, 44, 50, 60, 66, 70, or more amino acid residues. In some cases, the deletion is a N-terminal deletion, and the modified nucleoside triphosphate transporter comprises a deletion of about 5, 10, 15, 20, 22, 25, 30, 40, 44, 50, 60, 66, 70, or more amino acid residues at the N-terminus. In some cases, the modified nucleoside triphosphate transporter comprises a deletion of about 66 amino acid residues at the N-terminus.

In some instances, the isolated and modified nucleoside triphosphate transporter comprises at least 80%, 85%, 90%, 95%, 96%, 97%, 98%, or 99% sequence identity sequence identity to SEQ ID NOs: 4, 6, or 8. In some cases, the isolated and modified nucleoside triphosphate transporter comprises at least 80%, 85%, 90%, 95%, 96%, 97%, 98%, or 99% sequence identity sequence identity to SEQ ID NO: 4. In some cases, the isolated and modified nucleoside triphosphate transporter comprises 100% sequence identity to SEQ ID NO: 4. In some cases, the isolated and modified nucleoside triphosphate transporter comprises at least 80%, 85%, 90%, 95%, 96%, 97%, 98%, or 99% sequence identity to SEQ ID NO: 6. In some cases, the isolated and modified nucleoside triphosphate transporter comprises 100% sequence identity to SEQ ID NO: 6. In some cases, the isolated and modified nucleoside triphosphate transporter comprises at least 80%, 85%, 90%, 95%, 96%, 97%, 98%, or 99% sequence identity to SEQ ID NO: 8. In some cases, the isolated and modified nucleoside triphosphate transporter comprises 100% sequence identity to SEQ ID NO: 8.

In some embodiments, the modified nucleoside triphosphate transporter is under the control of a promoter. In some instances, the promoter is derived from an E. coli source. In other instances, the promoter is derived from a phage source. Exemplary promoters, include, but are not limited to, P_{bla}, P_{lac}, P_{lacUV5}, P_{H207}, Pλ, P_{tac}, or P_{N25}. In some instances, the promoter replaces the lac operon. In some cases, the modified nucleoside triphosphate transporter is under the control of a promoter selected from P_{bla}, P_{lac}, P_{lacUV5}, P_{H207}, Pλ, P_{tac}, or P_{N25}. In some cases, the modified nucleoside triphosphate transporter is under the control of promoter P_{lacUV5}.

In some instances, the modified nucleoside triphosphate transporter is encoded within a pSC plasmid.

In some embodiments, the engineered cell further comprises a second nucleic acid molecule encoding a Cas9 polypeptide or variants thereof, a third nucleic acid molecule encoding a single guide RNA (sgRNA) comprising a crRNA-tracrRNA scaffold; and a fourth nucleic acid molecule comprising an unnatural nucleotide.

The CRISPR/Cas system involves (1) an integration of short regions of genetic material that are homologous to a nucleic acid molecule of interest comprising an unnatural nucleotide, called “spacers”, in clustered arrays in the host genome, (2) expression of short guiding RNAs (crRNAs) from the spacers, (3) binding of the crRNAs to specific portions of the nucleic acid molecule of interest referred to as protospacers, and (4) degradation of protospacers by CRISPR-associated nucleases (Cas). In some cases, a Type-II CRISPR system has been described in the bacterium Streptococcus pyogenes, in which Cas9 and two non-coding small RNAs (pre-crRNA and tracrRNA (trans-activating CRISPR RNA)) act in concert to target and degrade a nucleic acid molecule of interest in a sequence-specific manner (Jinek et al., “A Programmable Dual-RNA-Guided DNA Endonuclease in Adaptive Bacterial Immunity,” Science 337(6096):816-821 (August 2012, epub Jun. 28, 2012)).

In some instances, a CRISPR/Cas system utilizes a Cas9 polypeptide or a variant thereof. Cas9 is a double stranded nuclease with two active cutting sites, one for each strand of the double helix. In some instances, the Cas9 polypeptide or variants thereof generate a double-stranded break. In some cases, the Cas9 polypeptide is a wild-type Cas9. In some instances, the Cas9 polypeptide is an optimized Cas9 for expression in an engineered cell described herein.

In some instances, the two noncoding RNAs are further fused into one single guide RNA (sgRNA). In some instances, the sgRNA comprises a target motif that recognizes a modification at the unnatural nucleotide position within a nucleic acid molecule of interest. In some embodiments, the modification is a substitution, insertion, or deletion. In some cases, the sgRNA comprises a target motif that recognizes a substitution at the unnatural nucleotide position within a nucleic acid molecule of interest. In some cases, the sgRNA comprises a target motif that recognizes a deletion at the unnatural nucleotide position within a nucleic acid molecule of interest. In some cases, the sgRNA comprises a target motif that recognizes an insertion at the unnatural nucleotide position within a nucleic acid molecule of interest.

In some cases, the target motif is between 10 to 30 nucleotides in length. In some instances, the target motif is between 15 to 30 nucleotides in length. In some cases, the target motif is about 11, 12, 13, 14, 15, 16, 17, 18, 19, 20, 21, 22, 23, 24, 25, 26, 27, 28, 29, or 30 nucleotides in length. In some cases, the target motif is about 15, 16, 17, 18, 19, 20, 21, or 22 nucleotides in length.

In some cases, the sgRNA further comprises a protospacer adjacent motif (PAM) recognition element. In some instances, PAM is located adjacent to the 3′ terminus of the target motif. In some cases, a nucleotide within the target motif that forms Watson-Crick base pairing with the modification at the unnatural nucleotide position within the nucleic acid molecule of interest is located between 3 to 22, between 5 to 20, between 5 to 18, between 5 to 15, between 5 to 12, or between 5 to 10 nucleotides from the 5′ terminus of PAM. In some cases, a nucleotide within the target motif that forms Watson-Crick base pairing with the modification at the unnatural nucleotide position within the nucleic acid molecule of interest is located about 3, 4, 5, 6, 7, 8, 9, 10, 11, 12, 13, 14, or 15 nucleotides from the 5′ terminus of PAM.

In some instances, the second nucleic acid molecule, the third nucleic acid molecule, and the fourth nucleic acid molecule are encoded in one or more plasmids. In some instances, the sgRNA encoded by the third nucleic acid molecule comprises a target motif that recognizes a modification at the unnatural nucleotide position within the fourth nucleic acid molecule. In some cases, the modification at the unnatural nucleotide position within the third nucleic acid molecule generates a modified third nucleic acid molecule. In some cases, the modification is a substitution, a deletion, or an insertion. In some cases, the sgRNA encoded by the third nucleic acid molecule further comprises a protospacer adjacent motif (PAM) recognition element. In some cases, the PAM element is adjacent to the 3′ terminus of the target motif. In some cases, the combination of Cas9 polypeptide or variants thereof and sgRNA modulates replication of the modified fourth nucleic acid molecule. In some cases, the combination of Cas9 polypeptide or variants thereof, sgRNA and the modified nucleoside triphosphate transporter modulates replication of the modified fourth nucleic acid molecule.

In some cases, the engineered cell further comprises an additional nucleic acid molecule that encodes an additional single guide RNA (sgRNA) comprising a crRNA-tracrRNA scaffold.

In some instances, the combination of Cas9 polypeptide or variants thereof, sgRNA and the modified nucleoside triphosphate transporter decreases the replication rate of the modified fourth nucleic acid molecule by about 80%, 85%, 95%, 99%, or higher. In some instances, the production of the fourth nucleic acid molecule in the engineered cell increases by about 50%, 60%, 70%, 80%, 90%, 95%, 96%, 97%, 98%, 99%, or higher.

In some embodiments, the modified nucleoside triphosphate transporter is expressed in a modified host strain (e.g., the engineered cell), optimized for the expression and activity of the modified nucleoside triphosphate transporter and/or general uptake of nucleoside triphosphates, natural or non-natural. For example, the expression of outer membrane porins, including, but not limited to, OmpA, OmpF, OmpC, may be modified, or the modified nucleoside triphosphate transporter may be expressed in the host cell (e.g., the engineered cell) that also expresses a heterologous outer membrane porin. Alternatively, the host cell (e.g., the engineered cell) may be permeabilized (chemically or by genetic means) to improve the uptake of nucleoside triphosphates. In some embodiments, the host cell (e.g., the engineered cell) may contain deletions of non-essential, endogenously secreted proteins to improve the capacity of the host secretion machinery for expression of the modified nucleoside triphosphate transporter.

In some embodiments, the modified nucleoside triphosphate transporter decreases doubling time of the host cell (e.g., the engineered cell).

In some cases, the modified nucleoside triphosphate transporter enables unnatural base pair retention of about 50%, 60%, 70%, 80%, 90%, 95%, 99%, or more.

### Plasmids Encoding a Modified Nucleoside Triphosphate Transporter

In some embodiments, also described herein is an isolated and purified plasmid comprising a nucleic acid molecule encoding a modified nucleoside triphosphate transporter from Phaeodactylum tricornutum (PtNTT2); and a promoter region selected from a pSC plasmid or lacZYA locus.

In some instances, the modified nucleoside triphosphate transporter is a codon optimized nucleoside triphosphate transporter from Phaeodactylum tricornutum.

In some instances, the modified nucleoside triphosphate transporter comprises a deletion. In some cases, the deletion is a terminal deletion or an internal deletion. In some cases, the deletion is a N-terminal truncation, a C-terminal truncation, or a truncation of both termini.

In some embodiments, the modified nucleoside triphosphate transporter comprises a deletion of about 5, 10, 15, 20, 22, 25, 30, 40, 44, 50, 60, 66, 70, or more amino acid residues. In some instances, the deletion is a N-terminal deletion. In some cases, the modified nucleoside triphosphate transporter comprises a deletion of about 5, 10, 15, 20, 22, 25, 30, 40, 44, 50, 60, 66, 70, or more amino acid residues at the N-terminus. In some cases, the modified nucleoside triphosphate transporter comprises a deletion of about 66 amino acid residues at the N-terminus.

In some instances, the isolated and modified nucleoside triphosphate transporter comprises at least 80%, 85%, 90%, 95%, 96%, 97%, 98%, or 99% sequence identity sequence identity to SEQ ID NOs: 4, 6, or 8. In some cases, the isolated and modified nucleoside triphosphate transporter comprises at least 80%, 85%, 90%, 95%, 96%, 97%, 98%, or 99% sequence identity sequence identity to SEQ ID NO: 4. In some cases, the isolated and modified nucleoside triphosphate transporter comprises 100% sequence identity to SEQ ID NO: 4. In some cases, the isolated and modified nucleoside triphosphate transporter comprises at least 80%, 85%, 90%, 95%, 96%, 97%, 98%, or 99% sequence identity to SEQ ID NO: 6. In some cases, the isolated and modified nucleoside triphosphate transporter comprises 100% sequence identity to SEQ ID NO: 6. In some cases, the isolated and modified nucleoside triphosphate transporter comprises at least 80%, 85%, 90%, 95%, 96%, 97%, 98%, or 99% sequence identity to SEQ ID NO: 8. In some cases, the isolated and modified nucleoside triphosphate transporter comprises 100% sequence identity to SEQ ID NO: 8.

In some embodiments, the modified nucleoside triphosphate transporter is under the control of a promoter. Exemplary promoters, include, but are not limited to, P_{bla}, P_{lac}, P_{lacUV5}, P_{H207}, Pλ, P_{tac}, or P_{N25}. In some instances, the promoter replaces the lac operon. In some cases, the modified nucleoside triphosphate transporter is under the control of a promoter selected from P_{bla}, P_{lac}, P_{lacUV5}, P_{H207}, Pλ, P_{tac}, or P_{N25}. In some cases, the modified nucleoside triphosphate transporter is under the control of promoter P_{lacUV5}.

In some instances, the modified nucleoside triphosphate transporter is encoded within a pSC plasmid.

In some embodiments, also disclosed herein is an in vivo method of increasing the production of a nucleic acid molecule containing an unnatural nucleotide comprising incubating a cell with an isolated and purified plasmid described supra.

### Nucleic Acid Molecules

In some embodiments, a nucleic acid (e.g., also referred to herein as nucleic acid molecule of interest) is from any source or composition, such as DNA, cDNA, gDNA (genomic DNA), RNA, siRNA (short inhibitory RNA), RNAi, tRNA, mRNA or rRNA (ribosomal RNA), for example, and is in any form (e.g., linear, circular, supercoiled, single-stranded, double-stranded, and the like). In some embodiments, nucleic acids comprise nucleotides, nucleosides, or polynucleotides. In some cases, nucleic acids comprise natural and unnatural nucleic acids. In some cases, a nucleic acid also comprises unnatural nucleic acids, such as DNA or RNA analogs (e.g., containing base analogs, sugar analogs and/or a non-native backbone and the like). It is understood that the term “nucleic acid” does not refer to or infer a specific length of the polynucleotide chain, thus polynucleotides and oligonucleotides are also included in the definition. Exemplary natural nucleotides include, without limitation, ATP, UTP, CTP, GTP, ADP, UDP, CDP, GDP, AMP, UMP, CMP, GMP, dATP, dTTP, dCTP, dGTP, dADP, dTDP, dCDP, dGDP, dAMP, dTMP, dCMP, and dGMP. Exemplary natural deoxyribonucleotides include dATP, dTTP, dCTP, dGTP, dADP, dTDP, dCDP, dGDP, dAMP, dTMP, dCMP, and dGMP. Exemplary natural ribonucleotides include ATP, UTP, CTP, GTP, ADP, UDP, CDP, GDP, AMP, UMP, CMP, and GMP. For RNA, the uracil base is uridine. A nucleic acid sometimes is a vector, plasmid, phagemid, autonomously replicating sequence (ARS), centromere, artificial chromosome, yeast artificial chromosome (e.g., YAC) or other nucleic acid able to replicate or be replicated in a host cell. In some cases, an unnatural nucleic acid is a nucleic acid analogue. In additional cases, an unnatural nucleic acid is from an extracellular source. In other cases, an unnatural nucleic acid is available to the intracellular space of an organism provided herein, e.g., a genetically modified organism.

### Unnatural Nucleic Acids

A nucleotide analog, or unnatural nucleotide, comprises a nucleotide which contains some type of modification to either the base, sugar, or phosphate moieties. In some embodiments, a modification comprises a chemical modification. In some cases, modifications occur at the 3′OH or 5′OH group, at the backbone, at the sugar component, or at the nucleotide base. Modifications, in some instances, optionally include non-naturally occurring linker molecules and/or of interstrand or intrastrand cross links. In one aspect, the modified nucleic acid comprises modification of one or more of the 3′OH or 5′OH group, the backbone, the sugar component, or the nucleotide base, and/or addition of non-naturally occurring linker molecules. In one aspect, a modified backbone comprises a backbone other than a phosphodiester backbone. In one aspect, a modified sugar comprises a sugar other than deoxyribose (in modified DNA) or other than ribose (modified RNA). In one aspect, a modified base comprises a base other than adenine, guanine, cytosine or thymine (in modified DNA) or a base other than adenine, guanine, cytosine or uracil (in modified RNA).

In some embodiments, the nucleic acid comprises at least one modified base. In some instances, the nucleic acid comprises 2, 3, 4, 5, 6, 7, 8, 9, 10, 15, 20, or more modified bases. In some cases, modifications to the base moiety include natural and synthetic modifications of A, C, G, and T/U as well as different purine or pyrimidine bases. In some embodiments, a modification is to a modified form of adenine, guanine cytosine or thymine (in modified DNA) or a modified form of adenine, guanine cytosine or uracil (modified RNA).

A modified base of a unnatural nucleic acid includes, but is not limited to, uracil-5-yl, hypoxanthin-9-yl (I), 2-aminoadenin-9-yl, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifiuoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Certain unnatural nucleic acids, such as 5-substituted pyrimidines, 6-azapyrimidines and N-2 substituted purines, N-6 substituted purines, 0-6 substituted purines, 2-aminopropyladenine, 5-propynyluracil, 5-propynylcytosine, 5-methylcytosine, those that increase the stability of duplex formation, universal nucleic acids, hydrophobic nucleic acids, promiscuous nucleic acids, size-expanded nucleic acids, fluorinated nucleic acids, 5-substituted pyrimidines, 6-azapyrimidines and N-2, N-6 and O—6 substituted purines, including 2-aminopropyladenine, 5-propynyluracil and 5-propynylcytosine. 5-methylcytosine (5-me-C), 5-hydroxymethyl cytosine, xanthine, hypoxanthine, 2-aminoadenine, 6-methyl, other alkyl derivatives of adenine and guanine, 2-propyl and other alkyl derivatives of adenine and guanine, 2-thiouracil, 2-thiothymine and 2-thiocytosine, 5-halouracil, 5-halocytosine, 5-propynyl (—C≡C—CI¼) uracil, 5-propynyl cytosine, other alkynyl derivatives of pyrimidine nucleic acids, 6-azo uracil, 6-azo cytosine, 6-azo thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl, other 5-substituted uracils and cytosines, 7-methylguanine, 7-methyladenine, 2-F-adenine, 2-amino-adenine, 8-azaguanine, 8-azaadenine, 7-deazaguanine, 7-deazaadenine, 3-deazaguanine, 3-deazaadenine, tricyclic pyrimidines, phenoxazine cytidine([5,4-b][1,4]benzoxazin-2(3H)-one), phenothiazine cytidine (1H— pyrimido[5,4-b][1,4]benzothiazin-2(3H)-one), G-clamps, phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one), those in which the purine or pyrimidine base is replaced with other heterocycles, 7-deaza-adenine, 7-deazaguanosine, 2-aminopyridine, 2-pyridone, azacytosine, 5-bromocytosine, bromouracil, 5-chlorocytosine, chlorinated cytosine, cyclocytosine, cytosine arabinoside, 5-fluorocytosine, fluoropyrimidine, fluorouracil, 5,6-dihydrocytosine, 5-iodocytosine, hydroxyurea, iodouracil, 5-nitrocytosine, 5-bromouracil, 5-chlorouracil, 5-fluorouracil, and 5-iodouracil, 2-amino-adenine, 6-thio-guanine, 2-thio-thymine, 4-thio-thymine, 5-propynyl-uracil, 4-thio-uracil, N4-ethylcytosine, 7-deazaguanine, 7-deaza-8-azaguanine, 5-hydroxycytosine, 2′-deoxyuridine, 2-amino-2′-deoxyadenosine, and those described in U.S. Pat. Nos. 3,687,808; 4,845,205; 4,910,300; 4,948,882; 5,093,232; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121; 5,596,091; 5,614,617; 5,645,985; 5,681,941; 5,750,692; 5,763,588; 5,830,653 and 6,005,096; WO 99/62923; Kandimalla et al., (2001) Bioorg. Med. Chem. 9:807-813; The Concise Encyclopedia of Polymer Science and Engineering, Kroschwitz, J. I., Ed., John Wiley & Sons, 1990, 858-859; Englisch et al., Angewandte Chemie, International Edition, 1991, 30, 613; and Sanghvi, Chapter 15, Antisense Research and Applications, Crookeand Lebleu Eds., CRC Press, 1993, 273-288. Additional base modifications can be found, for example, in U.S. Pat. No. 3,687,808; Englisch et al., Angewandte Chemie, International Edition, 1991, 30, 613; and Sanghvi, Chapter 15, Antisense Research and Applications, pages 289-302, Crooke and Lebleu ed., CRC Press, 1993.

Unnatural nucleic acids comprising various heterocyclic bases and various sugar moieties (and sugar analogs) are available in the art, and the nucleic acid in some cases include one or several heterocyclic bases other than the principal five base components of naturally-occurring nucleic acids. For example, the heterocyclic base includes, in some cases, uracil-5-yl, cytosin-5-yl, adenin-7-yl, adenin-8-yl, guanin-7-yl, guanin-8-yl, 4-aminopyrrolo[2.3-d]pyrimidin-5-yl, 2-amino-4-oxopyrolo[2,3-d]pyrimidin-5-yl, 2-amino-4-oxopyrrolo[2.3-d]pyrimidin-3-yl groups, where the purines are attached to the sugar moiety of the nucleic acid via the 9-position, the pyrimidines via the 1-position, the pyrrolopyrimidines via the 7-position and the pyrazolopyrimidines via the 1-position.

In some embodiments, a modified base of a unnatural nucleic acid is depicted below, wherein the wavy line identifies a point of attachment to the (deoxy)ribose or ribose.

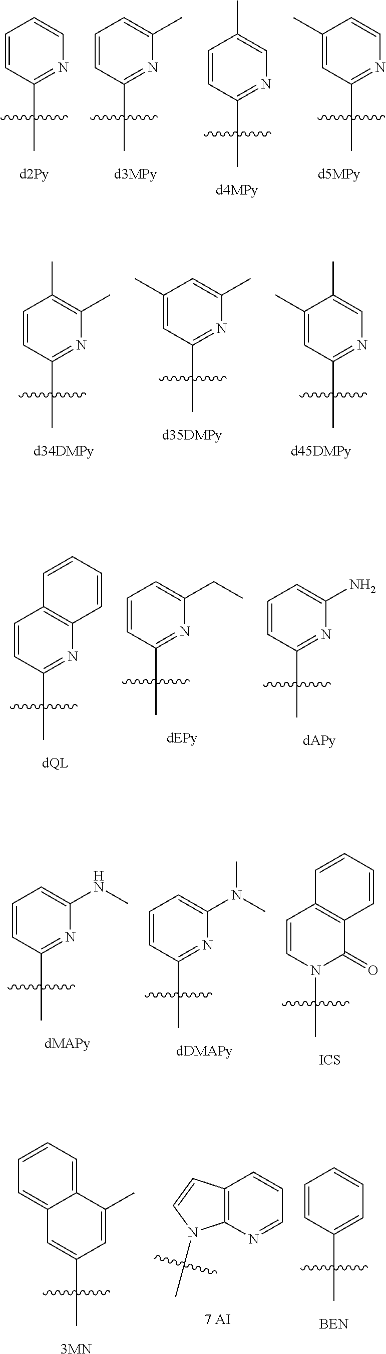

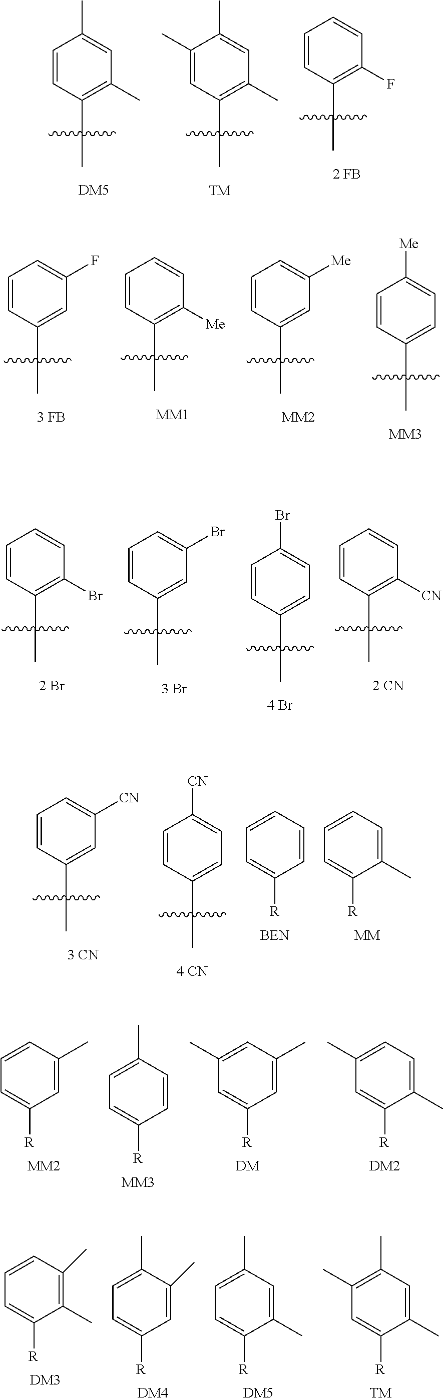

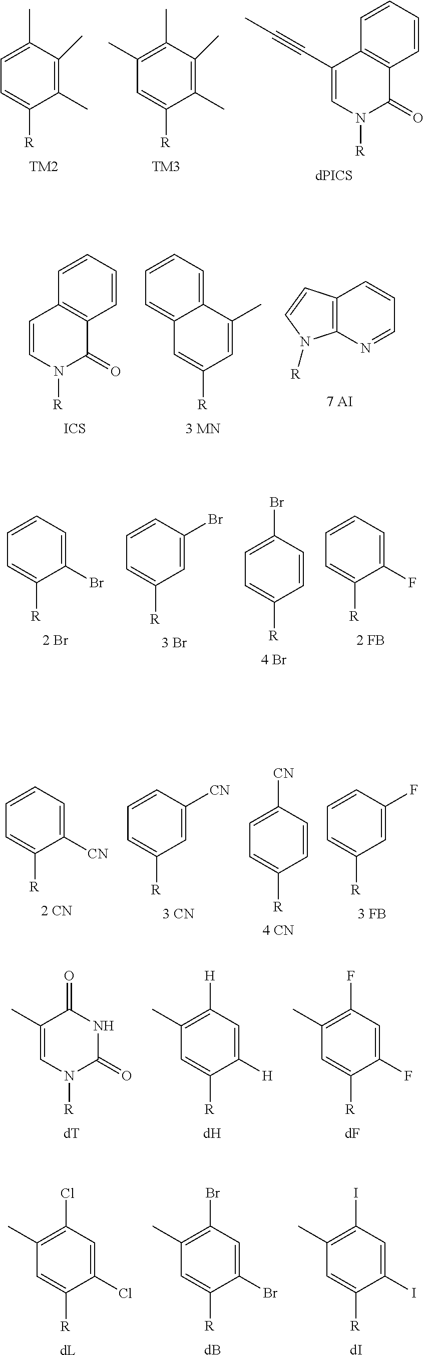

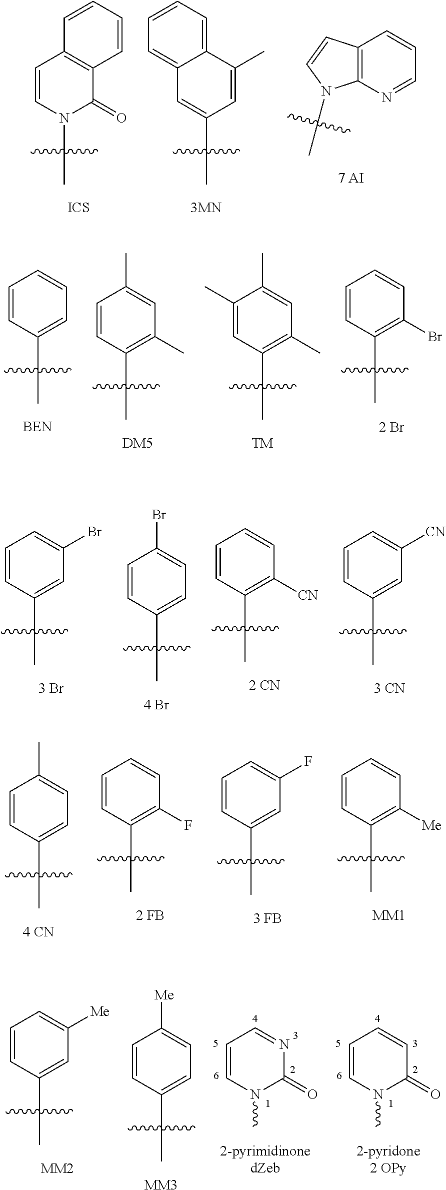

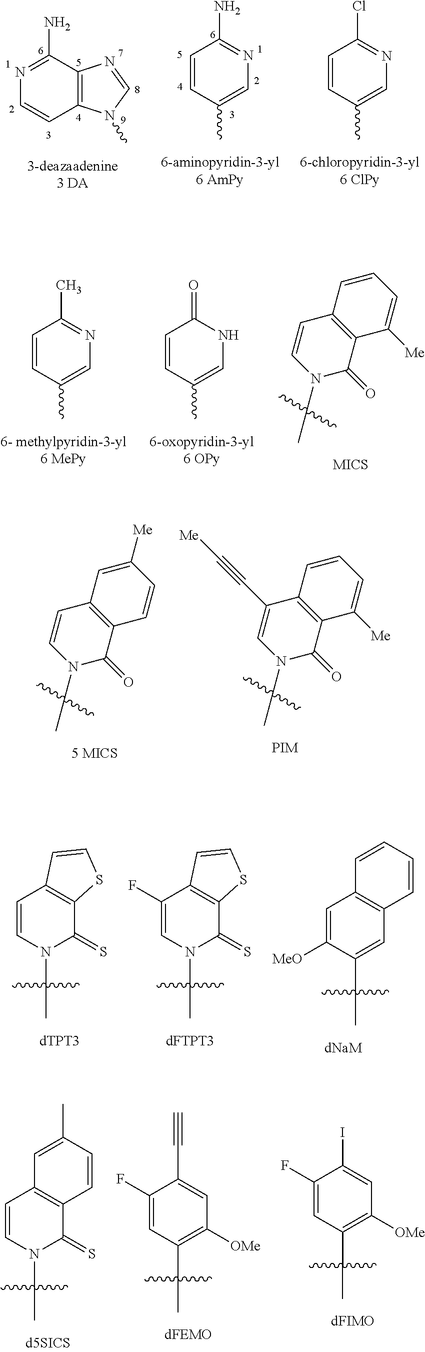

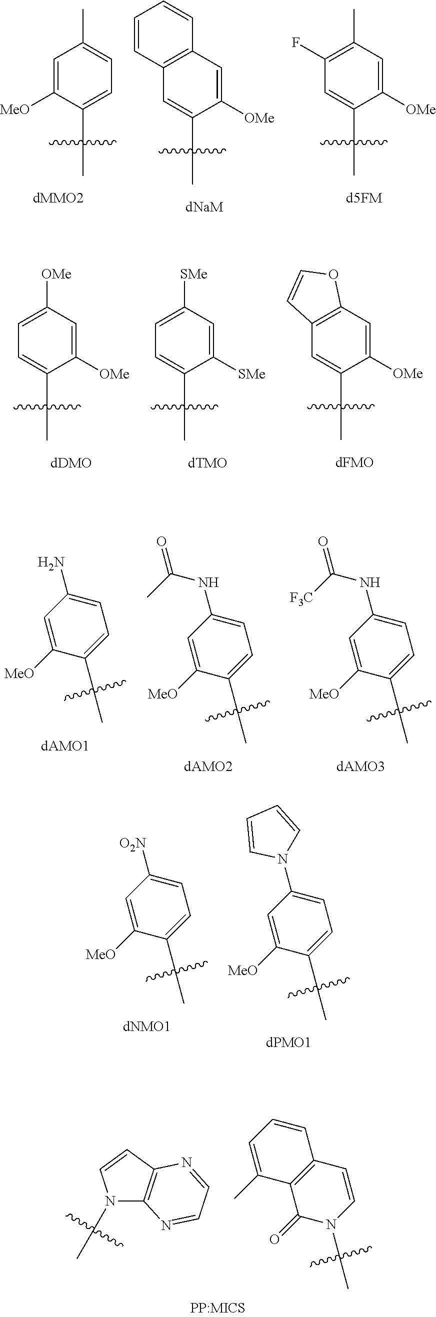

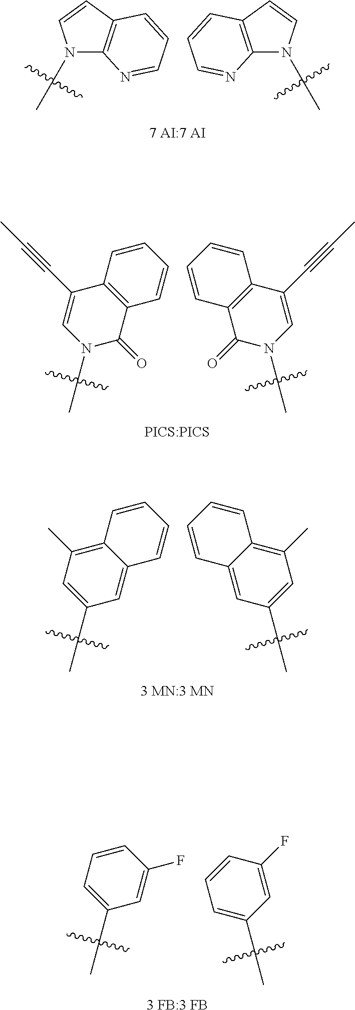

In some embodiments, nucleotide analogs are also modified at the phosphate moiety. Modified phosphate moieties include, but are not limited to, those with modification at the linkage between two nucleotides and contains, for example, a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. It is understood that these phosphate or modified phosphate linkage between two nucleotides are through a 3′-5′ linkage or a 2′-5′ linkage, and the linkage contains inverted polarity such as 3′-5′ to 5′-3′ or 2′-5′ to 5′-2′. Various salts, mixed salts and free acid forms are also included. Numerous United States patents teach how to make and use nucleotides containing modified phosphates and include but are not limited to, 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

In some embodiments, unnatural nucleic acids include 2′,3′-dideoxy-2′,3′-didehydro-nucleosides (PCT/US2002/006460), 5′-substituted DNA and RNA derivatives (PCT/US2011/033961; Saha et al., J. Org Chem., 1995, 60, 788-789; Wang et al., Bioorganic & Medicinal Chemistry Letters, 1999, 9, 885-890; and Mikhailov et al., Nucleosides & Nucleotides, 1991, 10(1-3), 339-343; Leonid et al., 1995, 14(3-5), 901-905; and Eppacher et al., Helvetica Chimica Acta, 2004, 87, 3004-3020; PCT/JP2000/004720; PCT/JP2003/002342; PCT/JP2004/013216; PCT/JP2005/020435; PCT/JP2006/315479; PCT/JP2006/324484; PCT/JP2009/056718; PCT/JP2010/067560), or 5′-substituted monomers made as the monophosphate with modified bases (Wang et al., Nucleosides Nucleotides & Nucleic Acids, 2004, 23 (1 & 2), 317-337).

In some embodiments, unnatural nucleic acids include modifications at the 5′-position and the 2′-position of the sugar ring (PCT/US94/02993), such as 5′-CH₂-substituted 2′-0-protected nucleosides (Wu et al., Helvetica Chimica Acta, 2000, 83, 1127-1143 and Wu et al., Bioconjugate Chem. 1999, 10, 921-924). In some cases, unnatural nucleic acids include amide linked nucleoside dimers have been prepared for incorporation into oligonucleotides wherein the 3′ linked nucleoside in the dimer (5′ to 3′) comprises a 2′-OCH₃ and a 5′—(S)—CH₃ (Mesmaeker et al., Synlett, 1997, 1287-1290). Unnatural nucleic acids can include 2′-substituted 5′-CH₂ (or O) modified nucleosides (PCT/US92/01020). Unnatural nucleic acids can include 5′-methylenephosphonate DNA and RNA monomers, and dimers (Bohringer et al., Tet. Lett., 1993, 34, 2723-2726; Collingwood et al., Synlett, 1995, 7, 703-705; and Hutter et al., Helvetica Chimica Acta, 2002, 85, 2777-2806). Unnatural nucleic acids can include 5′-phosphonate monomers having a 2′-substitution (US2006/0074035) and other modified 5′-phosphonate monomers (WO1997/35869). Unnatural nucleic acids can include 5′-modified methylenephosphonate monomers (EP614907 and EP629633). Unnatural nucleic acids can include analogs of 5′ or 6′-phosphonate ribonucleosides comprising a hydroxyl group at the 5′ and/or 6′-position (Chen et al., Phosphorus, Sulfur and Silicon, 2002, 777, 1783-1786; Jung et al., Bioorg. Med. Chem., 2000, 8, 2501-2509; Gallier et al., Eur. J. Org. Chem., 2007, 925-933; and Hampton et al., J. Med. Chem., 1976, 19(8), 1029-1033). Unnatural nucleic acids can include 5′-phosphonate deoxyribonucleoside monomers and dimers having a 5′-phosphate group (Nawrot et al., Oligonucleotides, 2006, 16(1), 68-82). Unnatural nucleic acids can include nucleosides having a 6′-phosphonate group wherein the 5′ or/and 6′-position is unsubstituted or substituted with a thio-tert-butyl group (SC(CH₃)₃) (and analogs thereof); a methyleneamino group (CH₂NH₂) (and analogs thereof) or a cyano group (CN) (and analogs thereof) (Fairhurst et al., Synlett, 2001, 4, 467-472; Kappler et al., J. Med. Chem., 1986, 29, 1030-1038; Kappler et al., J. Med. Chem., 1982, 25, 1179-1184; Vrudhula et al., J. Med. Chem., 1987, 30, 888-894; Hampton et al., J. Med. Chem., 1976, 19, 1371-1377; Geze et al., J. Am. Chem. Soc, 1983, 105(26), 7638-7640; and Hampton et al., J. Am. Chem. Soc, 1973, 95(13), 4404-4414).

In some embodiments, unnatural nucleic acids also include modifications of the sugar moiety. In some cases, nucleic acids contain one or more nucleosides wherein the sugar group has been modified. Such sugar modified nucleosides may impart enhanced nuclease stability, increased binding affinity, or some other beneficial biological property. In certain embodiments, nucleic acids comprise a chemically modified ribofuranose ring moiety. Examples of chemically modified ribofuranose rings include, without limitation, addition of substitutent groups (including 5′ and/or 2′ substituent groups; bridging of two ring atoms to form bicyclic nucleic acids (BNA); replacement of the ribosyl ring oxygen atom with S, N(R), or C(Ri)(R₂) (R=H, C₁-C₁₂ alkyl or a protecting group); and combinations thereof. Examples of chemically modified sugars can be found in WO2008/101157, US2005/0130923, and WO2007/134181.

In some instances, a modified nucleic acid comprises modified sugars or sugar analogs. Thus, in addition to ribose and deoxyribose, the sugar moiety can be pentose, deoxypentose, hexose, deoxyhexose, glucose, arabinose, xylose, lyxose, or a sugar “analog” cyclopentyl group. The sugar can be in a pyranosyl or furanosyl form. The sugar moiety may be the furanoside of ribose, deoxyribose, arabinose or 2′-O-alkylribose, and the sugar can be attached to the respective heterocyclic bases either in [alpha] or [beta]anomeric configuration. Sugar modifications include, but are not limited to, 2′-alkoxy-RNA analogs, 2′-amino-RNA analogs, 2′-fluoro-DNA, and 2′-alkoxy- or amino-RNA/DNA chimeras. For example, a sugar modification may include 2′-O-methyl-uridine or 2′-O-methyl-cytidine. Sugar modifications include 2′-O-alkyl-substituted deoxyribonucleosides and 2′-O-ethyleneglycol like ribonucleosides. The preparation of these sugars or sugar analogs and the respective “nucleosides” wherein such sugars or analogs are attached to a heterocyclic base (nucleic acid base) is known. Sugar modifications may also be made and combined with other modifications.

Modifications to the sugar moiety include natural modifications of the ribose and deoxy ribose as well as unnatural modifications. Sugar modifications include, but are not limited to, the following modifications at the 2′ position: OH; F; O—, S—, or N—alkyl; O—, S—, or N—alkenyl; O—, S— or N—alkynyl; or O—alkyl—O—alkyl, wherein the alkyl, alkenyl and alkynyl may be substituted or unsubstituted C₁ to C₁₀, alkyl or C₂ to C₁₀ alkenyl and alkynyl. 2′ sugar modifications also include but are not limited to —O[(CH₂)ₙO]ₘ CH₃, —O(CH₂)ₙ OCH₃, —O(CH₂)ₙNH₂, —O(CH₂)ₙCH₃, —O(CH₂)ONH₂, and —O(CH₂)ₙ ON[(CH₂)ₙ CH₃)]₂, where n and m are from 1 to about 10.

Other modifications at the 2′ position include but are not limited to: C₁ to C₁₀ lower alkyl, substituted lower alkyl, alkaryl, aralkyl, O-alkaryl, O-aralkyl, SH, SCH₃, OCN, Cl, Br, CN, CF₃, OCF₃, SOCH₃, SO₂CH₃, ONO₂, NO₂, N₃,NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of the 5′ terminal nucleotide. Modified sugars also include those that contain modifications at the bridging ring oxygen, such as CH₂ and S. Nucleotide sugar analogs may also have sugar mimetics such as cyclobutyl moieties in place of the pentofuranosyl sugar. There are numerous United States patents that teach the preparation of such modified sugar structures and which detail and describe a range of base modifications, such as U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; and 5,700,920, each of which is herein incorporated by reference in its entirety.

Examples of nucleic acids having modified sugar moieties include, without limitation, nucleic acids comprising 5′-vinyl, 5′-methyl (R or S), 4′-S, 2′-F, 2′-OCH₃, and 2′-O(CH₂)₂OCH₃ substituent groups. The substituent at the 2′ position can also be selected from allyl, amino, azido, thio, O-allyl, O—(C₁-C₁₀ alkyl), OCF₃, O(CH₂)₂SCH₃, O(CH₂)₂—O—N(Rₘ)(Rₙ), and O—CH₂—C(═O)—N(Rₘ)(Rₙ), where each Rₘ and Rₐ is, independently, H or substituted or unsubstituted C₁-C₁₀ alkyl.

In certain embodiments, nucleic acids described herein include one or more bicyclic nucleic acids. In certain such embodiments, the bicyclic nucleic acid comprises a bridge between the 4′ and the 2′ ribosyl ring atoms. In certain embodiments, nucleic acids provided herein include one or more bicyclic nucleic acids wherein the bridge comprises a 4′ to 2′ bicyclic nucleic acid. Examples of such 4′ to 2′ bicyclic nucleic acids include, but are not limited to, one of the formulae: 4′—(CH₂)—O-2′ (LNA); 4′—(CH₂)—S-2′; 4′—(CH₂)₂—O-2′ (ENA); 4′-CH(CH₃)—O-2′ and 4′-CH(CH₂OCH₃)—O-2′, and analogs thereof (see, U.S. Pat. No. 7,399,845); 4′-C(CH₃)(CH₃)—O-2′ and analogs thereof, (see WO2009/006478, WO2008/150729, US2004/0171570, U.S. Pat. No. 7,427,672, Chattopadhyaya et al., J. Org. Chem., 209, 74, 118-134, and WO2008/154401). Also see, for example: Singh et al., Chem. Commun., 1998, 4, 455-456; Koshkin et al., Tetrahedron, 1998, 54, 3607-3630; Wahlestedt et al., Proc. Natl. Acad. Sci. U.S.A, 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007, 129(26) 8362-8379; Elayadi et al., Curr. Opinion Invens. Drugs, 2001, 2, 558-561; Braasch et al., Chem. Biol, 2001, 8, 1-7; Oram et al., Curr. Opinion Mol. Ther., 2001, 3, 239-243; U.S. Pat. Nos. 4,849,513; 5,015,733; 5,118,800; 5,118,802; 7,053,207; 6,268,490; 6,770,748; 6,794,499; 7,034,133; 6,525,191; 6,670,461; and 7,399,845; International Publication Nos. WO2004/106356, WO1994/14226, WO2005/021570, WO2007/090071, and WO2007/134181; U.S. Patent Publication Nos. US2004/0171570, US2007/0287831, and US2008/0039618; U.S. Provisional Application Nos. 60/989,574, 61/026,995, 61/026,998, 61/056,564, 61/086,231, 61/097,787, and 61/099,844; and International Applications Nos. PCT/US2008/064591, PCT US2008/066154, PCT US2008/068922, and PCT/DK98/00393.

In certain embodiments, nucleic acids comprise linked nucleic acids. Nucleic acids can be linked together using any inter nucleic acid linkage. The two main classes of inter nucleic acid linking groups are defined by the presence or absence of a phosphorus atom. Representative phosphorus containing inter nucleic acid linkages include, but are not limited to, phosphodiesters, phosphotriesters, methylphosphonates, phosphoramidate, and phosphorothioates (P=S). Representative non-phosphorus containing inter nucleic acid linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N*-dimethylhydrazine (—CH₂—N(CH₃)—N(CH₃)). In certain embodiments, inter nucleic acids linkages having a chiral atom can be prepared as a racemic mixture, as separate enantiomers, e.g., alkylphosphonates and phosphorothioates. Unnatural nucleic acids can contain a single modification. Unnatural nucleic acids can contain multiple modifications within one of the moieties or between different moieties.

Backbone phosphate modifications to nucleic acid include, but are not limited to, methyl phosphonate, phosphorothioate, phosphoramidate (bridging or non-bridging), phosphotriester, phosphorodithioate, phosphodithioate, and boranophosphate, and may be used in any combination. Other non-phosphate linkages may also be used.

In some embodiments, backbone modifications (e.g., methylphosphonate, phosphorothioate, phosphoroamidate and phosphorodithioate internucleotide linkages) can confer immunomodulatory activity on the modified nucleic acid and/or enhance their stability in vivo.

In some instances, a phosphorous derivative (or modified phosphate group) is attached to the sugar or sugar analog moiety in and can be a monophosphate, diphosphate, triphosphate, alkylphosphonate, phosphorothioate, phosphorodithioate, phosphoramidate or the like. Exemplary polynucleotides containing modified phosphate linkages or non-phosphate linkages can be found in Peyrottes et al., 1996, Nucleic Acids Res. 24: 1841-1848; Chaturvedi et al., 1996, Nucleic Acids Res. 24:2318-2323; and Schultz et al., (1996) Nucleic Acids Res. 24:2966-2973; Matteucci, 1997, “Oligonucleotide Analogs: an Overview” in Oligonucleotides as Therapeutic Agents, (Chadwick and Cardew, ed.) John Wiley and Sons, New York, NY; Zon, 1993, “Oligonucleoside Phosphorothioates” in Protocols for Oligonucleotides and Analogs, Synthesis and Properties, Humana Press, pp. 165-190; Miller et al., 1971, JACS 93:6657-6665; Jager et al., 1988, Biochem. 27:7247-7246; Nelson et al., 1997, JOC 62:7278-7287; U.S. Pat. No. 5,453,496; and Micklefield, 2001, Curr. Med. Chem. 8: 1157-1179.

In some cases, backbone modification comprises replacing the phosphodiester linkage with an alternative moiety such as an anionic, neutral or cationic group. Examples of such modifications include: anionic internucleoside linkage; N3′ to P5′ phosphoramidate modification; boranophosphate DNA; prooligonucleotides; neutral internucleoside linkages such as methylphosphonates; amide linked DNA; methylene(methylimino) linkages; formacetal and thioformacetal linkages; backbones containing sulfonyl groups; morpholino oligos; peptide nucleic acids (PNA); and positively charged deoxyribonucleic guanidine (DNG) oligos (Micklefield, 2001, Current Medicinal Chemistry 8: 1157-1179). A modified nucleic acid may comprise a chimeric or mixed backbone comprising one or more modifications, e.g. a combination of phosphate linkages such as a combination of phosphodiester and phosphorothioate linkages.

Substitutes for the phosphate include, for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Numerous United States patents disclose how to make and use these types of phosphate replacements and include but are not limited to U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439. It is also understood in a nucleotide substitute that both the sugar and the phosphate moieties of the nucleotide can be replaced, by for example an amide type linkage (aminoethylglycine) (PNA). U.S. Pat. Nos. 5,539,082; 5,714,331; and 5,719,262 teach how to make and use PNA molecules, each of which is herein incorporated by reference. See also Nielsen et al., Science, 1991, 254, 1497-1500. It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. KY. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EM50J, 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1-di-O-hexadecyl-rac-glycero-S—H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochem. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937). Numerous United States patents teach the preparation of such conjugates and include, but are not limited to U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

### Nucleic Acid Base Pairing Properties

In some embodiments, an unnatural nucleic acid forms a base pair with another nucleic acid. In some embodiments, a stably integrated unnatural nucleic acid is an unnatural nucleic acid that can form a base pair with another nucleic acid, e.g., a natural or unnatural nucleic acid. In some embodiments, a stably integrated unnatural nucleic acid is an unnatural nucleic acid that can form a base pair with another unnatural nucleic acid (unnatural nucleic acid base pair (UBP)). For example, a first unnatural nucleic acid can form a base pair with a second unnatural nucleic acid. For example, one pair of unnatural nucleotide triphosphates that can base pair when incorporated into nucleic acids include a triphosphate of d5SICS (d5SICSTP) and a triphosphate of dNaM (dNaMTP). Such unnatural nucleotides can have a ribose or deoxyribose sugar moiety. In some embodiments, an unnatural nucleic acid does not substantially form a base pair with a natural nucleic acid (A, T, G, C). In some embodiments, a stably integrated unnatural nucleic acid can form a base pair with a natural nucleic acid.

In some embodiments, a stably integrated unnatural nucleic acid is an unnatural nucleic acid that can form a UBP, but does not substantially form a base pair with each of the four natural nucleic acids. In some embodiments, a stably integrated unnatural nucleic acid is an unnatural nucleic acid that can form a UBP, but does not substantially form a base pair with one or more natural nucleic acids. For example, a stably integrated unnatural nucleic acid may not substantially form a base pair with A, T, and, C, but can form a base pair with G. For example, a stably integrated unnatural nucleic acid may not substantially form a base pair with A, T, and, G, but can form a base pair with C. For example, a stably integrated unnatural nucleic acid may not substantially form a base pair with C, G, and, A, but can form a base pair with T. For example, a stably integrated unnatural nucleic acid may not substantially form a base pair with C, G, and, T, but can form a base pair with A. For example, a stably integrated unnatural nucleic acid may not substantially form a base pair with A and T, but can form a base pair with C and G. For example, a stably integrated unnatural nucleic acid may not substantially form a base pair with A and C, but can form a base pair with T and G. For example, a stably integrated unnatural nucleic acid may not substantially form a base pair with A and G, but can form a base pair with C and T. For example, a stably integrated unnatural nucleic acid may not substantially form a base pair with C and T, but can form a base pair with A and G. For example, a stably integrated unnatural nucleic acid may not substantially form a base pair with C and G, but can form a base pair with T and G. For example, a stably integrated unnatural nucleic acid may not substantially form a base pair with T and G, but can form a base pair with A and G. For example, a stably integrated unnatural nucleic acid may not substantially form a base pair with, G, but can form a base pair with A, T, and, C. For example, a stably integrated unnatural nucleic acid may not substantially form a base pair with, A, but can form a base pair with G, T, and, C. For example, a stably integrated unnatural nucleic acid may not substantially form a base pair with, T, but can form a base pair with G, A, and, C. For example, a stably integrated unnatural nucleic acid may not substantially form a base pair with, C, but can form a base pair with G, T, and, A.

Exemplary, unnatural nucleotides capable of forming an unnatural DNA or RNA base pair (UBP) under conditions in vivo includes, but is not limited to, 5SICS, d5SICS, NAM, dNaM, and combinations thereof. In some embodiments, unnatural nucleotides include:

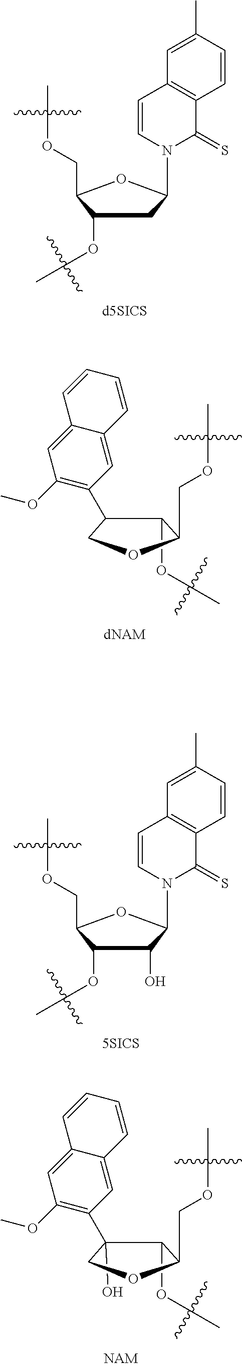

### Polymerase

A particularly useful function of a polymerase is to catalyze the polymerization of a nucleic acid strand using an existing nucleic acid as a template. Other functions that are useful are described elsewhere herein. Examples of useful polymerases include DNA polymerases and RNA polymerases.

The ability to improve specificity, processivity, or other features of polymerases unnatural nucleic acids would be highly desirable in a variety of contexts where, e.g., unnatural nucleic acid incorporation is desired, including amplification, sequencing, labeling, detection, cloning, and many others. The present invention provides polymerases with modified properties for unnatural nucleic acids, methods of making such polymerases, methods of using such polymerases, and many other features that will become apparent upon a complete review of the following.

In some instances, disclosed herein includes polymerases that incorporate unnatural nucleic acids into a growing template copy, e.g., during DNA amplification. In some embodiments, polymerases can be modified such that the active site of the polymerase is modified to reduce steric entry inhibition of the unnatural nucleic acid into the active site. In some embodiments, polymerases can be modified to provide complementarity with one or more unnatural features of the unnatural nucleic acids. Such polymerases can be expressed or engineered in cells for stably incorporating a UBP into the cells. Accordingly, the invention includes compositions that include a heterologous or recombinant polymerase and methods of use thereof.

Polymerases can be modified using methods pertaining to protein engineering. For example, molecular modeling can be carried out based on crystal structures to identify the locations of the polymerases where mutations can be made to modify a target activity. A residue identified as a target for replacement can be replaced with a residue selected using energy minimization modeling, homology modeling, and/or conservative amino acid substitutions, such as described in Bordo, et al. J Mol Biol 217: 721-729 (1991) and Hayes, et al. Proc Natl Acad Sci, USA 99: 15926-15931 (2002).

Any of a variety of polymerases can be used in a method or composition set forth herein including, for example, protein-based enzymes isolated from biological systems and functional variants thereof. Reference to a particular polymerase, such as those exemplified below, will be understood to include functional variants thereof unless indicated otherwise. In some embodiments, a polymerase is a wild type polymerase. In some embodiments, a polymerase is a modified, or mutant, polymerase.

Polymerases, with features for improving entry of unnatural nucleic acids into active site regions and for coordinating with unnatural nucleotides in the active site region, can also be used. In some embodiments, a modified polymerase has a modified nucleotide binding site.

In some embodiments, a modified polymerase has a specificity for an unnatural nucleic acid that is at least about 10%, 20%, 30%, 40%, 50%, 60%, 70%, 80%, 90%, 95%, 97%, 98%, 99%, 99.5%, 99.99% the specificity of the wild type polymerase toward the unnatural nucleic acid. In some embodiments, a modified or wild type polymerase has a specificity for an unnatural nucleic acid comprising a modified sugar that is at least about 10%, 20%, 30%, 40%, 50%, 60%, 70%, 80%, 90%, 95%, 97%, 98%, 99%, 99.5%, 99.99% the specificity of the wild type polymerase toward a natural nucleic acid and/or the unnatural nucleic acid without the modified sugar. In some embodiments, a modified or wild type polymerase has a specificity for an unnatural nucleic acid comprising a modified base that is at least about 10%, 20%, 30%, 40%, 50%, 60%, 70%, 80%, 90%, 95%, 97%, 98%, 99%, 99.5%, 99.99% the specificity of the wild type polymerase toward a natural nucleic acid and/or the unnatural nucleic acid without the modified base. In some embodiments, a modified or wild type polymerase has a specificity for an unnatural nucleic acid comprising a triphosphate that is at least about 10%, 20%, 30%, 40%, 50%, 60%, 70%, 80%, 90%, 95%, 97%, 98%, 99%, 99.5%, 99.99% the specificity of the wild type polymerase toward a nucleic acid comprising a triphosphate and/or the unnatural nucleic acid without the triphosphate. For example, a modified or wild type polymerase can have a specificity for an unnatural nucleic acid comprising a triphosphate that is at least about 10%, 20%, 30%, 40%, 50%, 60%, 70%, 80%, 90%, 95%, 97%, 98%, 99%, 99.5% 99.99% the specificity of the wild type polymerase toward the unnatural nucleic acid with a diphosphate or monophosphate, or no phosphate, or a combination thereof.

In some embodiments, a modified or wild type polymerase has a relaxed specificity for an unnatural nucleic acid. In some embodiments, a modified or wild type polymerase has a specificity for an unnatural nucleic acid and a specificity to a natural nucleic acid that is at least about 10%, 20%, 30%, 40%, 50%, 60%, 70%, 80%, 90%, 95%, 97%, 98%, 99%, 99.5%, 99.99% the specificity of the wild type polymerase toward the natural nucleic acid. In some embodiments, a modified or wild type polymerase has a specificity for an unnatural nucleic acid comprising a modified sugar and a specificity to a natural nucleic acid that is at least about 10%, 20%, 30%, 40%, 50%, 60%, 70%, 80%, 90%, 95%, 97%, 98%, 99%, 99.5% 99.99% the specificity of the wild type polymerase toward the natural nucleic acid. In some embodiments, a modified or wild type polymerase has a specificity for an unnatural nucleic acid comprising a modified base and a specificity to a natural nucleic acid that is at least about 10%, 20%, 30%, 40%, 50%, 60%, 70%, 80%, 90%, 95%, 97%, 98%, 99%, 99.5%, 99.99% the specificity of the wild type polymerase toward the natural nucleic acid.

Absence of exonuclease activity can be a wild type characteristic or a characteristic imparted by a variant or engineered polymerase. For example, an exo minus Klenow fragment is a mutated version of Klenow fragment that lacks 3′ to 5′ proofreading exonuclease activity.

The method of the invention may be used to expand the substrate range of any DNA polymerase which lacks an intrinsic 3 to 5′ exonuclease proofreading activity or where a 3 to 5′ exonuclease proofreading activity has been disabled, e.g. through mutation. Examples of DNA polymerases include polA, polB (see e.g. Parrel & Loeb, Nature Struc Biol 2001) polC, polD, polY, polX and reverse transcriptases (RT) but preferably are processive, high-fidelity polymerases (PCT/GB2004/004643). In some embodiments a modified or wild type polymerase substantially lacks 3′ to 5′ proofreading exonuclease activity. In some embodiments a modified or wild type polymerase substantially lacks 3′ to 5′ proofreading exonuclease activity for an unnatural nucleic acid. In some embodiments, a modified or wild type polymerase has a 3′ to 5′ proofreading exonuclease activity. In some embodiments, a modified or wild type polymerase has a 3′ to 5′ proofreading exonuclease activity for a natural nucleic acid and substantially lacks 3′ to 5′ proofreading exonuclease activity for an unnatural nucleic acid.

In some embodiments, a modified polymerase has a 3′ to 5′ proofreading exonuclease activity that is at least about 60%, 70%, 80%, 90%, 95%, 97%, 98%, 99%, 99.5%, 99.99% the proofreading exonuclease activity of the wild type polymerase. In some embodiments, a modified polymerase has a 3′ to 5′ proofreading exonuclease activity for an unnatural nucleic acid that is at least about 60%, 70%, 80%, 90%, 95%, 97%, 98%, 99%, 99.5%, 99.99% the proofreading exonuclease activity of the wild type polymerase to a natural nucleic acid. In some embodiments, a modified polymerase has a 3′ to 5′ proofreading exonuclease activity for an unnatural nucleic acid and a 3′ to 5′ proofreading exonuclease activity for a natural nucleic acid that is at least about 60%, 70%, 80%, 90%, 95%, 97%, 98%, 99%, 99.5%, 99.99% the proofreading exonuclease activity of the wild type polymerase to a natural nucleic acid. In some embodiments, a modified polymerase has a 3′ to 5′ proofreading exonuclease activity for a natural nucleic acid that is at least about 60%, 70%, 80%, 90%, 95%, 97%, 98%, 99%, 99.5%, 99.99% the proofreading exonuclease activity of the wild type polymerase to the natural nucleic acid.

In some embodiments, polymerases are characterized according to their rate of dissociation from nucleic acids. In some embodiments a polymerase has a relatively low dissociation rate for one or more natural and unnatural nucleic acids. In some embodiments a polymerase has a relatively high dissociation rate for one or more natural and unnatural nucleic acids. The dissociation rate is an activity of a polymerase that can be adjusted to tune reaction rates in methods set forth herein.

In some embodiments, polymerases are characterized according to their fidelity when used with a particular natural and/or unnatural nucleic acid or collections of natural and/or unnatural nucleic acid. Fidelity generally refers to the accuracy with which a polymerase incorporates correct nucleic acids into a growing nucleic acid chain when making a copy of a nucleic acid template. DNA polymerase fidelity can be measured as the ratio of correct to incorrect natural and unnatural nucleic acid incorporations when the natural and unnatural nucleic acid are present, e.g., at equal concentrations, to compete for strand synthesis at the same site in the polymerase-strand-template nucleic acid binary complex. DNA polymerase fidelity can be calculated as the ratio of (k_{cat}/Kₘ) for the natural and unnatural nucleic acid and (k_{cat}/Kₘ) for the incorrect natural and unnatural nucleic acid; where k_{cat} and Kₘ are Michaelis-Menten parameters in steady state enzyme kinetics (Fersht, A. R. (1985) Enzyme Structure and Mechanism, 2nd ed., p 350, W. H. Freeman & Co., New York., incorporated herein by reference). In some embodiments, a polymerase has a fidelity value of at least about 100, 1000, 10,000, 100,000, or 1×10⁶, with or without a proofreading activity.

In some embodiments, polymerases from native sources or variants thereof are screened using an assay that detects incorporation of an unnatural nucleic acid having a particular structure. In one example, polymerases can be screened for the ability to incorporate an unnatural nucleic acid or UBP; e.g., d5SICSTP, dNaMTP, or d5SICSTP-dNaMTP UBP. A polymerase, e.g., a heterologous polymerase, can be used that displays a modified property for the unnatural nucleic acid as compared to the wild-type polymerase. For example, the modified property can be, e.g., Kₘ, k_{cat}, Vₘₐₓ, polymerase processivity in the presence of an unnatural nucleic acid (or of a naturally occurring nucleotide), average template read-length by the polymerase in the presence of an unnatural nucleic acid, specificity of the polymerase for an unnatural nucleic acid, rate of binding of an unnatural nucleic acid, rate of product (pyrophosphate, triphosphate, etc.) release, branching rate, or any combination thereof. In one embodiment, the modified property is a reduced Kₘ for an unnatural nucleic acid and/or an increased k_{cat}/Kₘ or Vₘₐₓ/Kₘ for an unnatural nucleic acid. Similarly, the polymerase optionally has an increased rate of binding of an unnatural nucleic acid, an increased rate of product release, and/or a decreased branching rate, as compared to a wild-type polymerase.

At the same time, a polymerase can incorporate natural nucleic acids, e.g., A, C, G, and T, into a growing nucleic acid copy. For example, a polymerase optionally displays a specific activity for a natural nucleic acid that is at least about 5% as high (e.g., 5%, 10%, 25%, 50%, 75%, 100% or higher), as a corresponding wild-type polymerase and a processivity with natural nucleic acids in the presence of a template that is at least 5% as high (e.g., 5%, 10%, 25%, 50%, 75%, 100% or higher) as the wild-type polymerase in the presence of the natural nucleic acid. Optionally, the polymerase displays a k_{cat}/Kₘ or Vₘₐₓ/Kₘ for a naturally occurring nucleotide that is at least about 5% as high (e.g., about 5%, 10%, 25%, 50%, 75% or 100% or higher) as the wild-type polymerase.

Polymerases used herein that can have the ability to incorporate an unnatural nucleic acid of a particular structure can also be produced using a directed evolution approach. A nucleic acid synthesis assay can be used to screen for polymerase variants having specificity for any of a variety of unnatural nucleic acids. For example, polymerase variants can be screened for the ability to incorporate an unnatural nucleic acid or UBP; e.g., d5SICSTP, dNaMTP, or d5SICSTP-dNaMTP UBP into nucleic acids. In some embodiments, such an assay is an in vitro assay, e.g., using a recombinant polymerase variant. In some embodiments, such an assay is an in vivo assay, e.g., expressing a polymerase variant in a cell. Such directed evolution techniques can be used to screen variants of any suitable polymerase for activity toward any of the unnatural nucleic acids set forth herein.

Modified polymerases of the compositions described can optionally be a modified and/or recombinant Φ29-type DNA polymerase. Optionally, the polymerase can be a modified and/or recombinant ΦD29, B103, GA-1, PZA, ΦD15, BS32, M2Y, Nf, G1, Cp-1, PRD1, PZE, SF5, Cp-5, Cp-7, PR4, PR5, PR722, or L17 polymerase.

Nucleic acid polymerases generally useful in the invention include DNA polymerases, RNA polymerases, reverse transcriptases, and mutant or altered forms thereof. DNA polymerases and their properties are described in detail in, among other places, DNA Replication 2^{nd} edition, Kornberg and Baker, W. H. Freeman, New York, N. Y. (1991). Known conventional DNA polymerases useful in the invention include, but are not limited to, Pyrococcus furiosus (Pfu) DNA polymerase (Lundberg et al., 1991, Gene, 108: 1, Stratagene), Pyrococcus woesei (Pwo) DNA polymerase (Hinnisdaels et al., 1996, Biotechniques, 20:186-8, Boehringer Mannheim), Thermus thermophilus (Tth) DNA polymerase (Myers and Gelfand 1991, Biochemistry 30:7661), Bacillus stearothermophilus DNA polymerase (Stenesh and McGowan, 1977, Biochim Biophys Acta 475:32), Thermococcus litoralis (TIi) DNA polymerase (also referred to as Vent™ DNA polymerase, Cariello et al, 1991, Polynucleotides Res, 19: 4193, New England Biolabs), 9° Nm™ DNA polymerase (New England Biolabs), Stoffel fragment, Thermo Sequenase® (Amersham Pharmacia Biotech UK), Therminator™ (New England Biolabs), Thermotoga maritima (Tma) DNA polymerase (Diaz and Sabino, 1998 Braz J Med. Res, 31:1239), Thermus aquaticus (Taq) DNA polymerase (Chien et al, 1976, J. Bacteoriol, 127: 1550), DNA polymerase, Pyrococcus kodakaraensis KOD DNA polymerase (Takagi et al., 1997, Appl. Environ. Microbiol. 63:4504), JDF-3 DNA polymerase (from thermococcus sp. JDF-3, Patent application WO 0132887), Pyrococcus GB-D (PGB-D) DNA polymerase (also referred as Deep Vent™ DNA polymerase, Juncosa-Ginesta et al., 1994, Biotechniques, 16:820, New England Biolabs), UlTma DNA polymerase (from thermophile Thermotoga maritima; Diaz and Sabino, 1998 Braz J. Med. Res, 31:1239; PE Applied Biosystems), Tgo DNA polymerase (from thermococcus gorgonarius, Roche Molecular Biochemicals), E. coli DNA polymerase I (Lecomte and Doubleday, 1983, Polynucleotides Res. 11:7505), T7 DNA polymerase (Nordstrom et al, 1981, J Biol. Chem. 256:3112), and archaeal DP1I/DP2 DNA polymerase II (Cann et al, 1998, Proc. Natl. Acad. Sci. USA 95:14250). Both mesophilic polymerases and thermophilic polymerases are contemplated. Thermophilic DNA polymerases include, but are not limited to, ThermoSequenase®, 9° Nm™, Therminator™, Taq, Tne, Tma, Pfu, TfI, Tth, TIi, Stoffel fragment, Vent™ and Deep Vent™ DNA polymerase, KOD DNA polymerase, Tgo, JDF-3, and mutants, variants and derivatives thereof. A polymerase that is a 3′ exonuclease-deficient mutant is also contemplated. Reverse transcriptases useful in the invention include, but are not limited to, reverse transcriptases from HIV, HTLV-I, HTLV-II, FeLV, FIV, SIV, AMV, MMTV, MoMuLV and other retroviruses (see Levin, Cell 88:5-8 (1997); Verma, Biochim Biophys Acta. 473:1-38 (1977); Wu et al, CRC Crit Rev Biochem. 3:289-347(1975)). Further examples of polymerases include, but are not limited to 9° N DNA Polymerase, Taq DNA polymerase, Phusion® DNA polymerase, Pfu DNA polymerase, RB69 DNA polymerase, KOD DNA polymerase, and VentR® DNA polymerase Gardner et al. (2004) “Comparative Kinetics of Nucleotide Analog Incorporation by Vent DNA Polymerase (J. Biol. Chem., 279(12), 11834-11842; Gardner and Jack “Determinants of nucleotide sugar recognition in an archaeon DNA polymerase” Nucleic Acids Research, 27(12) 2545-2553.) Polymerases isolated from non-thermophilic organisms can be heat inactivatable. Examples are DNA polymerases from phage. It will be understood that polymerases from any of a variety of sources can be modified to increase or decrease their tolerance to high temperature conditions. In some embodiments, a polymerase can be thermophilic. In some embodiments, a thermophilic polymerase can be heat inactivatable. Thermophilic polymerases are typically useful for high temperature conditions or in thermocycling conditions such as those employed for polymerase chain reaction (PCR) techniques.

In some embodiments, the polymerase comprises (129, B103, GA-1, PZA, (D15, BS32, M2Y, Nf, G1, Cp-1, PRD1, PZE, SF5, Cp-5, Cp-7, PR4, PR5, PR722, L17, ThermoSequenase®, 9° Nm™, Therminator™ DNA polymerase, Tne, Tma, TfI, Tth, TIi, Stoffel fragment, Vent™ and Deep Vent™ DNA polymerase, KOD DNA polymerase, Tgo, JDF-3, Pfu, Taq, T7 DNA polymerase, T7 RNA polymerase, PGB-D, UlTma DNA polymerase, E. coli DNA polymerase I, E. coli DNA polymerase III, archaeal DP1II/DP2 DNA polymerase II, 9° N DNA Polymerase, Taq DNA polymerase, Phusion® DNA polymerase, Pfu DNA polymerase, SP6 RNA polymerase, RB69 DNA polymerase, Avian Myeloblastosis Virus (AMV) reverse transcriptase, Moloney Murine Leukemia Virus (MMLV) reverse transcriptase, SuperScript® II reverse transcriptase, and SuperScript® III reverse transcriptase.

In some embodiments, the polymerase is DNA polymerase 1-Klenow fragment, Vent polymerase, Phusion® DNA polymerase, KOD DNA polymerase, Taq polymerase, T7 DNA polymerase, T7 RNA polymerase, Therminator™ DNA polymerase, POLB polymerase, SP6 RNA polymerase, E. coli DNA polymerase I, E. coli DNA polymerase III, Avian Myeloblastosis Virus (AMV) reverse transcriptase, Moloney Murine Leukemia Virus (MMLV) reverse transcriptase, SuperScript® II reverse transcriptase, or SuperScript® III reverse transcriptase.

Additionally, such polymerases can be used for DNA amplification and/or sequencing applications, including real-time applications, e.g., in the context of amplification or sequencing that include incorporation of unnatural nucleic acid residues into DNA by the polymerase. In other embodiments, the unnatural nucleic acid that is incorporated can be the same as a natural residue, e.g., where a label or other moiety of the unnatural nucleic acid is removed by action of the polymerase during incorporation, or the unnatural nucleic acid can have one or more feature that distinguishes it from a natural nucleic acid.

### Nucleic Acid Reagents & Tools

A nucleic acid reagent for use with a method, cell, or engineered microorganism described herein comprises one or more ORFs. An ORF may be from any suitable source, sometimes from genomic DNA, mRNA, reverse transcribed RNA or complementary DNA (cDNA) or a nucleic acid library comprising one or more of the foregoing, and is from any organism species that contains a nucleic acid sequence of interest, protein of interest, or activity of interest. Non-limiting examples of organisms from which an ORF can be obtained include bacteria, yeast, fungi, human, insect, nematode, bovine, equine, canine, feline, rat or mouse, for example. In some embodiments, a nucleic acid reagent or other reagent described herein is isolated or purified.

A nucleic acid reagent sometimes comprises a nucleotide sequence adjacent to an ORF that is translated in conjunction with the ORF and encodes an amino acid tag. The tag-encoding nucleotide sequence is located 3′ and/or 5′ of an ORF in the nucleic acid reagent, thereby encoding a tag at the C-terminus or N-terminus of the protein or peptide encoded by the ORF. Any tag that does not abrogate in vitro transcription and/or translation may be utilized and may be appropriately selected by the artisan. Tags may facilitate isolation and/or purification of the desired ORF product from culture or fermentation media.

A nucleic acid or nucleic acid reagent can comprise certain elements, e.g., regulatory elements, often selected according to the intended use of the nucleic acid. Any of the following elements can be included in or excluded from a nucleic acid reagent. A nucleic acid reagent, for example, may include one or more or all of the following nucleotide elements: one or more promoter elements, one or more 5′ untranslated regions (5′UTRs), one or more regions into which a target nucleotide sequence may be inserted (an “insertion element”), one or more target nucleotide sequences, one or more 3′ untranslated regions (3′UTRs), and one or more selection elements. A nucleic acid reagent can be provided with one or more of such elements and other elements may be inserted into the nucleic acid before the nucleic acid is introduced into the desired organism. In some embodiments, a provided nucleic acid reagent comprises a promoter, 5′UTR, optional 3′UTR and insertion element(s) by which a target nucleotide sequence is inserted (i.e., cloned) into the nucleotide acid reagent. In certain embodiments, a provided nucleic acid reagent comprises a promoter, insertion element(s) and optional 3′UTR, and a 5′ UTR/target nucleotide sequence is inserted with an optional 3′UTR. The elements can be arranged in any order suitable for expression in the chosen expression system (e.g., expression in a chosen organism, or expression in a cell free system, for example), and in some embodiments a nucleic acid reagent comprises the following elements in the 5′ to 3′ direction: (1) promoter element, 5′UTR, and insertion element(s); (2) promoter element, 5′UTR, and target nucleotide sequence; (3) promoter element, 5′UTR, insertion element(s) and 3′UTR; and (4) promoter element, 5′UTR, target nucleotide sequence and 3′UTR.

Nucleic acid reagents, e.g., expression cassettes and/or expression vectors, can include a variety of regulatory elements, including promoters, enhancers, translational initiation sequences, transcription termination sequences and other elements. A “promoter” is generally a sequence or sequences of DNA that function when in a relatively fixed location in regard to the transcription start site. For example, the promoter can be upstream of the nucleotide triphosphate transporter nucleic acid segment. A “promoter” contains core elements required for basic interaction of RNA polymerase and transcription factors and can contain upstream elements and response elements. “Enhancer” generally refers to a sequence of DNA that functions at no fixed distance from the transcription start site and can be either 5′ or 3″ to the transcription unit. Furthermore, enhancers can be within an intron as well as within the coding sequence itself. They are usually between 10 and 300 by in length, and they function in cis. Enhancers function to increase transcription from nearby promoters. Enhancers, like promoters, also often contain response elements that mediate the regulation of transcription. Enhancers often determine the regulation of expression.

As noted above, nucleic acid reagents may also comprise one or more 5′ UTR's, and one or more 3′UTR's. For example, expression vectors used in prokaryotic host cells (e.g., virus, bacterium) can contain sequences that signal for the termination of transcription which can affect mRNA expression. These regions can be transcribed as polyadenylated segments in the untranslated portion of the mRNA encoding tissue factor protein. The 3″ untranslated regions also include transcription termination sites. In some preferred embodiments, a transcription unit comprises a polyadenylation region. One benefit of this region is that it increases the likelihood that the transcribed unit will be processed and transported like mRNA. The identification and use of polyadenylation signals in expression constructs is well established. In some preferred embodiments, homologous polyadenylation signals can be used in the transgene constructs.

A 5′ UTR may comprise one or more elements endogenous to the nucleotide sequence from which it originates, and sometimes includes one or more exogenous elements. A 5′ UTR can originate from any suitable nucleic acid, such as genomic DNA, plasmid DNA, RNA or mRNA, for example, from any suitable organism (e.g., virus, bacterium, yeast, fungi, plant, insect or mammal). The artisan may select appropriate elements for the 5′ UTR based upon the chosen expression system (e.g., expression in a chosen organism, or expression in a cell free system, for example). A 5′ UTR sometimes comprises one or more of the following elements known to the artisan: enhancer sequences (e.g., transcriptional or translational), transcription initiation site, transcription factor binding site, translation regulation site, translation initiation site, translation factor binding site, accessory protein binding site, feedback regulation agent binding sites, Pribnow box, TATA box, −35 element, E-box (helix-loop-helix binding element), ribosome binding site, replicon, internal ribosome entry site (IRES), silencer element and the like. In some embodiments, a promoter element may be isolated such that all 5′ UTR elements necessary for proper conditional regulation are contained in the promoter element fragment, or within a functional subsequence of a promoter element fragment.

A 5′UTR in the nucleic acid reagent can comprise a translational enhancer nucleotide sequence. A translational enhancer nucleotide sequence often is located between the promoter and the target nucleotide sequence in a nucleic acid reagent. A translational enhancer sequence often binds to a ribosome, sometimes is an 18S rRNA-binding ribonucleotide sequence (i.e., a 40S ribosome binding sequence) and sometimes is an internal ribosome entry sequence (IRES). An IRES generally forms an RNA scaffold with precisely placed RNA tertiary structures that contact a 40S ribosomal subunit via a number of specific intermolecular interactions. Examples of ribosomal enhancer sequences are known and can be identified by the artisan (e.g., Mignone et al., Nucleic Acids Research 33: D141-D146 (2005); Paulous et al., Nucleic Acids Research 31: 722-733 (2003); Akbergenov et al., Nucleic Acids Research 32: 239-247 (2004); Mignone et al., Genome Biology 3(3): reviews0004.1-0001.10 (2002); Gallie, Nucleic Acids Research 30: 3401-3411 (2002); Shaloiko et al., DOI: 10.1002/bit.20267; and Gallie et al., Nucleic Acids Research 15: 3257-3273 (1987)).

A translational enhancer sequence sometimes is a eukaryotic sequence, such as a Kozak consensus sequence or other sequence (e.g., hydroid polyp sequence, GenBank accession no. U07128). A translational enhancer sequence sometimes is a prokaryotic sequence, such as a Shine-Dalgarno consensus sequence. In certain embodiments, the translational enhancer sequence is a viral nucleotide sequence. A translational enhancer sequence sometimes is from a 5′ UTR of a plant virus, such as Tobacco Mosaic Virus (TMV), Alfalfa Mosaic Virus (AMV); Tobacco Etch Virus (ETV); Potato Virus Y (PVY); Turnip Mosaic (poty) Virus and Pea Seed Borne Mosaic Virus, for example. In certain embodiments, an omega sequence about 67 bases in length from TMV is included in the nucleic acid reagent as a translational enhancer sequence (e.g., devoid of guanosine nucleotides and includes a 25 nucleotide long poly (CAA) central region).

A 3′ UTR may comprise one or more elements endogenous to the nucleotide sequence from which it originates and sometimes includes one or more exogenous elements. A 3′ UTR may originate from any suitable nucleic acid, such as genomic DNA, plasmid DNA, RNA or mRNA, for example, from any suitable organism (e.g., a virus, bacterium, yeast, fungi, plant, insect or mammal). The artisan can select appropriate elements for the 3′ UTR based upon the chosen expression system (e.g., expression in a chosen organism, for example). A 3′ UTR sometimes comprises one or more of the following elements known to the artisan: transcription regulation site, transcription initiation site, transcription termination site, transcription factor binding site, translation regulation site, translation termination site, translation initiation site, translation factor binding site, ribosome binding site, replicon, enhancer element, silencer element and polyadenosine tail. A 3′ UTR often includes a polyadenosine tail and sometimes does not, and if a polyadenosine tail is present, one or more adenosine moieties may be added or deleted from it (e.g., about 5, about 10, about 15, about 20, about 25, about 30, about 35, about 40, about 45 or about 50 adenosine moieties may be added or subtracted).

In some embodiments, modification of a 5′ UTR and/or a 3′ UTR is used to alter (e.g., increase, add, decrease or substantially eliminate) the activity of a promoter. Alteration of the promoter activity can in turn alter the activity of a peptide, polypeptide or protein (e.g., enzyme activity for example), by a change in transcription of the nucleotide sequence(s) of interest from an operably linked promoter element comprising the modified 5′ or 3′ UTR. For example, a microorganism can be engineered by genetic modification to express a nucleic acid reagent comprising a modified 5′ or 3′ UTR that can add a novel activity (e.g., an activity not normally found in the host organism) or increase the expression of an existing activity by increasing transcription from a homologous or heterologous promoter operably linked to a nucleotide sequence of interest (e.g., homologous or heterologous nucleotide sequence of interest), in certain embodiments. In some embodiments, a microorganism can be engineered by genetic modification to express a nucleic acid reagent comprising a modified 5′ or 3′ UTR that can decrease the expression of an activity by decreasing or substantially eliminating transcription from a homologous or heterologous promoter operably linked to a nucleotide sequence of interest, in certain embodiments.

Expression of a nucleotide triphosphate transporter from an expression cassette or expression vector can be controlled by any promoter capable of expression in prokaryotic cells. A promoter element typically is required for DNA synthesis and/or RNA synthesis. A promoter element often comprises a region of DNA that can facilitate the transcription of a particular gene, by providing a start site for the synthesis of RNA corresponding to a gene. Promoters generally are located near the genes they regulate, are located upstream of the gene (e.g., 5′ of the gene), and are on the same strand of DNA as the sense strand of the gene, in some embodiments. In some embodiments, a promoter element can be isolated from a gene or organism and inserted in functional connection with a polynucleotide sequence to allow altered and/or regulated expression. A non-native promoter (e.g., promoter not normally associated with a given nucleic acid sequence) used for expression of a nucleic acid often is referred to as a heterologous promoter. In certain embodiments, a heterologous promoter and/or a 5′UTR can be inserted in functional connection with a polynucleotide that encodes a polypeptide having a desired activity as described herein. The terms “operably linked” and “in functional connection with” as used herein with respect to promoters, refer to a relationship between a coding sequence and a promoter element. The promoter is operably linked or in functional connection with the coding sequence when expression from the coding sequence via transcription is regulated, or controlled by, the promoter element. The terms “operably linked” and “in functional connection with” are utilized interchangeably herein with respect to promoter elements.

A promoter often interacts with a RNA polymerase. A polymerase is an enzyme that catalyzes synthesis of nucleic acids using a preexisting nucleic acid reagent. When the template is a DNA template, an RNA molecule is transcribed before protein is synthesized. Enzymes having polymerase activity suitable for use in the present methods include any polymerase that is active in the chosen system with the chosen template to synthesize protein. In some embodiments, a promoter (e.g., a heterologous promoter) also referred to herein as a promoter element, can be operably linked to a nucleotide sequence or an open reading frame (ORF). Transcription from the promoter element can catalyze the synthesis of an RNA corresponding to the nucleotide sequence or ORF sequence operably linked to the promoter, which in turn leads to synthesis of a desired peptide, polypeptide or protein.

Promoter elements sometimes exhibit responsiveness to regulatory control. Promoter elements also sometimes can be regulated by a selective agent. That is, transcription from promoter elements sometimes can be turned on, turned off, up-regulated or down-regulated, in response to a change in environmental, nutritional or internal conditions or signals (e.g., heat inducible promoters, light regulated promoters, feedback regulated promoters, hormone influenced promoters, tissue specific promoters, oxygen and pH influenced promoters, promoters that are responsive to selective agents (e.g., kanamycin) and the like, for example). Promoters influenced by environmental, nutritional or internal signals frequently are influenced by a signal (direct or indirect) that binds at or near the promoter and increases or decreases expression of the target sequence under certain conditions.

Non-limiting examples of selective or regulatory agents that influence transcription from a promoter element used in embodiments described herein include, without limitation, (1) nucleic acid segments that encode products that provide resistance against otherwise toxic compounds (e.g., antibiotics); (2) nucleic acid segments that encode products that are otherwise lacking in the recipient cell (e.g., essential products, tRNA genes, auxotrophic markers); (3) nucleic acid segments that encode products that suppress the activity of a gene product; (4) nucleic acid segments that encode products that can be readily identified (e.g., phenotypic markers such as antibiotics (e.g., β-lactamase), 0-galactosidase, green fluorescent protein (GFP), yellow fluorescent protein (YFP), red fluorescent protein (RFP), cyan fluorescent protein (CFP), and cell surface proteins); (5) nucleic acid segments that bind products that are otherwise detrimental to cell survival and/or function; (6) nucleic acid segments that otherwise inhibit the activity of any of the nucleic acid segments described in Nos. 1-5 above (e.g., antisense oligonucleotides); (7) nucleic acid segments that bind products that modify a substrate (e.g., restriction endonucleases); (8) nucleic acid segments that can be used to isolate or identify a desired molecule (e.g., specific protein binding sites); (9) nucleic acid segments that encode a specific nucleotide sequence that can be otherwise non-functional (e.g., for PCR amplification of subpopulations of molecules); (10) nucleic acid segments that, when absent, directly or indirectly confer resistance or sensitivity to particular compounds; (11) nucleic acid segments that encode products that either are toxic or convert a relatively non-toxic compound to a toxic compound (e.g., Herpes simplex thymidine kinase, cytosine deaminase) in recipient cells; (12) nucleic acid segments that inhibit replication, partition or heritability of nucleic acid molecules that contain them; and/or (13) nucleic acid segments that encode conditional replication functions, e.g., replication in certain hosts or host cell strains or under certain environmental conditions (e.g., temperature, nutritional conditions, and the like). In some embodiments, the regulatory or selective agent can be added to change the existing growth conditions to which the organism is subjected (e.g., growth in liquid culture, growth in a fermenter, growth on solid nutrient plates and the like for example).

In some embodiments, regulation of a promoter element can be used to alter (e.g., increase, add, decrease or substantially eliminate) the activity of a peptide, polypeptide or protein (e.g., enzyme activity for example). For example, a microorganism can be engineered by genetic modification to express a nucleic acid reagent that can add a novel activity (e.g., an activity not normally found in the host organism) or increase the expression of an existing activity by increasing transcription from a homologous or heterologous promoter operably linked to a nucleotide sequence of interest (e.g., homologous or heterologous nucleotide sequence of interest), in certain embodiments. In some embodiments, a microorganism can be engineered by genetic modification to express a nucleic acid reagent that can decrease expression of an activity by decreasing or substantially eliminating transcription from a homologous or heterologous promoter operably linked to a nucleotide sequence of interest, in certain embodiments.

Nucleic acids encoding heterologous proteins, e.g., nucleotide triphosphate transporters, can be inserted into or employed with any suitable expression system. In some embodiments, a nucleic acid reagent sometimes is stably integrated into the chromosome of the host organism, or a nucleic acid reagent can be a deletion of a portion of the host chromosome, in certain embodiments (e.g., genetically modified organisms, where alteration of the host genome confers the ability to selectively or preferentially maintain the desired organism carrying the genetic modification). Such nucleic acid reagents (e.g., nucleic acids or genetically modified organisms whose altered genome confers a selectable trait to the organism) can be selected for their ability to guide production of a desired protein or nucleic acid molecule. When desired, the nucleic acid reagent can be altered such that codons encode for (i) the same amino acid, using a different tRNA than that specified in the native sequence, or (ii) a different amino acid than is normal, including unconventional or unnatural amino acids (including detectably labeled amino acids).

Recombinant expression is usefully accomplished using an expression cassette that can be part of a vector, such as a plasmid. A vector can include a promoter operably linked to nucleic acid encoding a nucleotide triphosphate transporter. A vector can also include other elements required for transcription and translation as described herein. An expression cassette, expression vector, and sequences in a cassette or vector can be heterologous to the cell to which the unnatural nucleotides are contacted. For example, a nucleotide triphosphate transporter sequence can be heterologous to the cell.

A variety of prokaryotic expression vectors suitable for carrying, encoding and/or expressing nucleotide triphosphate transporters can be produced. Such expression vectors include, for example, pET, pET3d, pCR2.1, pBAD, pUC, and yeast vectors. The vectors can be used, for example, in a variety of in vivo and in vitro situations. Non-limiting examples of prokaryotic promoters that can be used include SP6, T7, T5, tac, bla, trp, gal, lac, or maltose promoters. Viral vectors that can be employed include those relating to lentivirus, adenovirus, adeno-associated virus, herpes virus, vaccinia virus, polio virus, AIDS virus, neuronal trophic virus, Sindbis and other viruses. Also useful are any viral families which share the properties of these viruses which make them suitable for use as vectors. Retroviral vectors that can be employed include those described in Verma, American Society for Microbiology, pp. 229-232, Washington, (1985). For example, such retroviral vectors can include Murine Maloney Leukemia virus, MMLV, and other retroviruses that express desirable properties. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral nucleic acid.

### Cloning

Any convenient cloning strategy known in the art may be utilized to incorporate an element, such as an ORF, into a nucleic acid reagent. Known methods can be utilized to insert an element into the template independent of an insertion element, such as (1) cleaving the template at one or more existing restriction enzyme sites and ligating an element of interest and (2) adding restriction enzyme sites to the template by hybridizing oligonucleotide primers that include one or more suitable restriction enzyme sites and amplifying by polymerase chain reaction (described in greater detail herein). Other cloning strategies take advantage of one or more insertion sites present or inserted into the nucleic acid reagent, such as an oligonucleotide primer hybridization site for PCR, for example, and others described herein. In some embodiments, a cloning strategy can be combined with genetic manipulation such as recombination (e.g., recombination of a nucleic acid reagent with a nucleic acid sequence of interest into the genome of the organism to be modified, as described further herein). In some embodiments, the cloned ORF(s) can produce (directly or indirectly) modified or wild type nucleotide triphosphate transporters and/or polymerases), by engineering a microorganism with one or more ORFs of interest, which microorganism comprises altered activities of nucleotide triphosphate transporter activity or polymerase activity.

A nucleic acid may be specifically cleaved by contacting the nucleic acid with one or more specific cleavage agents. Specific cleavage agents often will cleave specifically according to a particular nucleotide sequence at a particular site. Examples of enzyme specific cleavage agents include without limitation endonucleases (e.g., DNase (e.g., DNase I, II); RNase (e.g., RNase E, F, H, P); Cleavase™ enzyme; Taq DNA polymerase; E. coli DNA polymerase I; murine FEN-1 endonucleases; type I, II or III restriction endonucleases such as Acc I, Afl III, Alu I, Alw44 I, Apa I, Asn I, Ava I, Ava II, BamH I, Ban II, Bcl I, Bgl I. Bgl II, Bln I, BsaI, Bsm I, BsmBI, BssH II, BstE II, Cfo I, CIa I, Dde I, Dpn I, Dra I, EcIX I, EcoR I, EcoR I, EcoR II, EcoR V, Hae II, Hae II, Hind II, Hind III, Hpa I, Hpa II, Kpn I, Ksp I, Mlu I, MIuN I, Msp I, Nci I, Nco I, Nde I, Nde II, Nhe I, Not I, Nru I, Nsi I, Pst I, Pvu I, Pvu II, Rsa I, Sac I, Sal I, Sau3A I, Sca I, ScrF I, Sfi I, Sma I, Spe I, Sph I, Ssp I, Stu I, Sty I, Swa I, Taq I, Xba I, Xho I); glycosylases (e.g., uracil-DNA glycolsylase (UDG), 3-methyladenine DNA glycosylase, 3-methyladenine DNA glycosylase II, pyrimidine hydrate-DNA glycosylase, FaPy-DNA glycosylase, thymine mismatch-DNA glycosylase, hypoxanthine-DNA glycosylase, 5-Hydroxymethyluracil DNA glycosylase (HmUDG), 5-Hydroxymethylcytosine DNA glycosylase, or 1,N6-etheno-adenine DNA glycosylase); exonucleases (e.g., exonuclease III); ribozymes, and DNAzymes. Sample nucleic acid may be treated with a chemical agent, or synthesized using modified nucleotides, and the modified nucleic acid may be cleaved. In non-limiting examples, sample nucleic acid may be treated with (i) alkylating agents such as methylnitrosourea that generate several alkylated bases, including N3-methyladenine and N3-methylguanine, which are recognized and cleaved by alkyl purine DNA-glycosylase; (ii) sodium bisulfite, which causes deamination of cytosine residues in DNA to form uracil residues that can be cleaved by uracil N-glycosylase; and (iii) a chemical agent that converts guanine to its oxidized form, 8-hydroxyguanine, which can be cleaved by formamidopyrimidine DNA N-glycosylase. Examples of chemical cleavage processes include without limitation alkylation, (e.g., alkylation of phosphorothioate-modified nucleic acid); cleavage of acid lability of P3′—N5′-phosphoroamidate-containing nucleic acid; and osmium tetroxide and piperidine treatment of nucleic acid.

In some embodiments, the nucleic acid reagent includes one or more recombinase insertion sites. A recombinase insertion site is a recognition sequence on a nucleic acid molecule that participates in an integration/recombination reaction by recombination proteins. For example, the recombination site for Cre recombinase is loxP, which is a 34 base pair sequence comprised of two 13 base pair inverted repeats (serving as the recombinase binding sites) flanking an 8 base pair core sequence (e.g., Sauer, Curr. Opin. Biotech. 5:521-527 (1994)). Other examples of recombination sites include attB, attP, attL, and attR sequences, and mutants, fragments, variants and derivatives thereof, which are recognized by the recombination protein λ Int and by the auxiliary proteins integration host factor (IHF), FIS and excisionase (Xis) (e.g., U.S. Pat. Nos. 5,888,732; 6,143,557; 6,171,861; 6,270,969; 6,277,608; and 6,720,140; U.S. patent application Ser. Nos. 09/517,466, and 09/732,914; U.S. Patent Publication No. US2002/0007051; and Landy, Curr. Opin. Biotech. 3:699-707 (1993)).

Examples of recombinase cloning nucleic acids are in Gateway® systems (Invitrogen, California), which include at least one recombination site for cloning desired nucleic acid molecules in vivo or in vitro. In some embodiments, the system utilizes vectors that contain at least two different site-specific recombination sites, often based on the bacteriophage lambda system (e.g., att1 and att2), and are mutated from the wild-type (att0) sites. Each mutated site has a unique specificity for its cognate partner att site (i.e., its binding partner recombination site) of the same type (for example attB1 with attP1, or attL1 with attR1) and will not cross-react with recombination sites of the other mutant type or with the wild-type att0 site. Different site specificities allow directional cloning or linkage of desired molecules thus providing desired orientation of the cloned molecules. Nucleic acid fragments flanked by recombination sites are cloned and subcloned using the Gateway® system by replacing a selectable marker (for example, ccdB) flanked by att sites on the recipient plasmid molecule, sometimes termed the Destination Vector. Desired clones are then selected by transformation of a ccdB sensitive host strain and positive selection for a marker on the recipient molecule. Similar strategies for negative selection (e.g., use of toxic genes) can be used in other organisms such as thymidine kinase (TK) in mammals and insects.

A nucleic acid reagent sometimes contains one or more origin of replication (ORI) elements. In some embodiments, a template comprises two or more ORIs, where one functions efficiently in one organism (e.g., a bacterium) and another function efficiently in another organism (e.g., a eukaryote, like yeast for example). In some embodiments, an ORI may function efficiently in one species (e.g., S. cerevisiae, for example) and another ORI may function efficiently in a different species (e.g., S. pombe, for example). A nucleic acid reagent also sometimes includes one or more transcription regulation sites.

A nucleic acid reagent, e.g., an expression cassette or vector, can include nucleic acid sequence encoding a marker product. A marker product is used to determine if a gene has been delivered to the cell and once delivered is being expressed. Example marker genes include the E. coli lacZ gene which encodes P-galactosidase and green fluorescent protein. In some embodiments the marker can be a selectable marker. When such selectable markers are successfully transferred into a host cell, the transformed host cell can survive if placed under selective pressure. There are two widely used distinct categories of selective regimes. The first category is based on a cell's metabolism and the use of a mutant cell line which lacks the ability to grow independent of a supplemented media. The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin (Southern etal., J. Molec. Appl. Genet. 1: 327 (1982)), mycophenolic acid, (Mulligan et al.,. Science 209: 1422 (1980)) or hygromycin, (Sugden, et al., Mol. Cell. Biol. 5: 410-413 (1985)).

A nucleic acid reagent can include one or more selection elements (e.g., elements for selection of the presence of the nucleic acid reagent, and not for activation of a promoter element which can be selectively regulated). Selection elements often are utilized using known processes to determine whether a nucleic acid reagent is included in a cell. In some embodiments, a nucleic acid reagent includes two or more selection elements, where one functions efficiently in one organism, and another functions efficiently in another organism. Examples of selection elements include, but are not limited to, (1) nucleic acid segments that encode products that provide resistance against otherwise toxic compounds (e.g., antibiotics); (2) nucleic acid segments that encode products that are otherwise lacking in the recipient cell (e.g., essential products, tRNA genes, auxotrophic markers); (3) nucleic acid segments that encode products that suppress the activity of a gene product; (4) nucleic acid segments that encode products that can be readily identified (e.g., phenotypic markers such as antibiotics (e.g., P-lactamase), 0-galactosidase, green fluorescent protein (GFP), yellow fluorescent protein (YFP), red fluorescent protein (RFP), cyan fluorescent protein (CFP), and cell surface proteins); (5) nucleic acid segments that bind products that are otherwise detrimental to cell survival and/or function; (6) nucleic acid segments that otherwise inhibit the activity of any of the nucleic acid segments described in Nos. 1-5 above (e.g., antisense oligonucleotides); (7) nucleic acid segments that bind products that modify a substrate (e.g., restriction endonucleases); (8) nucleic acid segments that can be used to isolate or identify a desired molecule (e.g., specific protein binding sites); (9) nucleic acid segments that encode a specific nucleotide sequence that can be otherwise non-functional (e.g., for PCR amplification of subpopulations of molecules); (10) nucleic acid segments that, when absent, directly or indirectly confer resistance or sensitivity to particular compounds; (11) nucleic acid segments that encode products that either are toxic or convert a relatively non-toxic compound to a toxic compound (e.g., Herpes simplex thymidine kinase, cytosine deaminase) in recipient cells; (12) nucleic acid segments that inhibit replication, partition or heritability of nucleic acid molecules that contain them; and/or (13) nucleic acid segments that encode conditional replication functions, e.g., replication in certain hosts or host cell strains or under certain environmental conditions (e.g., temperature, nutritional conditions, and the like).

A nucleic acid reagent can be of any form useful for in vivo transcription and/or translation. A nucleic acid sometimes is a plasmid, such as a supercoiled plasmid, sometimes is a yeast artificial chromosome (e.g., YAC), sometimes is a linear nucleic acid (e.g., a linear nucleic acid produced by PCR or by restriction digest), sometimes is single-stranded and sometimes is double-stranded. A nucleic acid reagent sometimes is prepared by an amplification process, such as a polymerase chain reaction (PCR) process or transcription-mediated amplification process (TMA). In TMA, two enzymes are used in an isothermal reaction to produce amplification products detected by light emission (e.g., Biochemistry 1996 Jun 25;35(25):8429-38). Standard PCR processes are known (e.g., U.S. Pat. Nos. 4,683,202; 4,683,195; 4,965,188; and 5,656,493), and generally are performed in cycles. Each cycle includes heat denaturation, in which hybrid nucleic acids dissociate; cooling, in which primer oligonucleotides hybridize; and extension of the oligonucleotides by a polymerase (i.e., Taq polymerase). An example of a PCR cyclical process is treating the sample at 95° C. for 5 minutes; repeating forty-five cycles of 95° C. for 1 minute, 59° C. for 1 minute, 10 seconds, and 72° C. for 1 minute 30 seconds; and then treating the sample at 72° C. for 5 minutes. Multiple cycles frequently are performed using a commercially available thermal cycler. PCR amplification products sometimes are stored for a time at a lower temperature (e.g., at 4° C.) and sometimes are frozen (e.g., at −20° C.) before analysis.

### Kits/Article of Manufacture

Disclosed herein, in certain embodiments, are kits and articles of manufacture for use with one or more methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

In some embodiments, a kit includes a suitable packaging material to house the contents of the kit. In some cases, the packaging material is constructed by well-known methods, preferably to provide a sterile, contaminant-free environment. The packaging materials employed herein can include, for example, those customarily utilized in commercial kits sold for use with nucleic acid sequencing systems. Exemplary packaging materials include, without limitation, glass, plastic, paper, foil, and the like, capable of holding within fixed limits a component set forth herein.

The packaging material can include a label which indicates a particular use for the components. The use for the kit that is indicated by the label can be one or more of the methods set forth herein as appropriate for the particular combination of components present in the kit. For example, a label can indicate that the kit is useful for a method of synthesizing a polynucleotide or for a method of determining the sequence of a nucleic acid.

Instructions for use of the packaged reagents or components can also be included in a kit. The instructions will typically include a tangible expression describing reaction parameters, such as the relative amounts of kit components and sample to be admixed, maintenance time periods for reagent/sample admixtures, temperature, buffer conditions, and the like.

It will be understood that not all components necessary for a particular reaction need be present in a particular kit. Rather one or more additional components can be provided from other sources. The instructions provided with a kit can identify the additional component(s) that are to be provided and where they can be obtained.

In some embodiments, a kit is provided that is useful for stably incorporating an unnatural nucleic acid into a cellular nucleic acid, e.g., using the methods provided by the present invention for preparing genetically engineered cells. In one embodiment, a kit described herein includes a genetically engineered cell and one or more unnatural nucleic acids. In another embodiment, a kit described herein includes an isolated and purified plasmid comprising a sequence selected from SEQ ID NOs: 1-9. In a further embodiment, a kit described herein includes an isolated and purified plasmid comprises a sequence of SEQ ID NOs: 2, 3, 5, or 7.

In additional embodiments, the kit described herein provides a cell and a nucleic acid molecule containing a heterologous gene for introduction into the cell to thereby provide a genetically engineered cell, such as expression vectors comprising the nucleic acid of any of the embodiments hereinabove described in this paragraph.

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

The section headings used herein are for organizational purposes only and are not to be construed as limiting the subject matter described.

### EXAMPLES

These examples are provided for illustrative purposes only and not to limit the scope of the claims provided herein.

### Example 1

All natural organisms store genetic information in a four letter, and two base pair genetic alphabet. In some instances, a mutant form of Escherichia coli was generated, grown in the presence of the unnatural nucleoside triphosphates dNaMTP and d5SICSTP, and provided with the means to import them via expression of a plasmid-borne nucleoside triphosphate transporter, replicates DNA containing a single dNaM-d5SICS UBP. In some cases, the organism grew poorly, and was unable to indefinitely store the unnatural information, which is a prerequisite for true semi-synthetic life. Described below comprise an engineered transporter, coupled with a chemically optimized UBP, to generate a semi-synthetic organism (SSO).

### Methods

Unless otherwise stated, liquid bacterial cultures were grown in 2×YT (casein peptone 16 g/L, yeast extract 10 g/L, NaCl 5 g/L) supplemented with potassium phosphate (50 mM, pH 7), referred to hereafter as “media”, and incubated at 37° C. in a 48-well flat bottomed plate (CELLSTAR, Greiner Bio-One) with shaking at 200 rpm. Solid growth media was prepared with 2% agar. Antibiotics were used, as appropriate, at the following concentrations: carbenicillin, 100 μg/mL; streptomycin, 50 μg/mL; kanamycin, 50 μg/mL; zeocin, 50 μg/mL; chloramphenicol, 33 μg/mL for plasmids, 5 μg/mL for chromosomal integrants. All selective agents were purchased commercially. Cell growth, indicated as OD₆₀₀, was measured using a Perkin Elmer Envision 2103 Multilabel Reader with a 590/20 nm filter.

Unless otherwise stated, all molecular biology reagents were obtained from New England Biolabs (NEB) and were used according to the manufacturer's protocols. PCRs for cloning and strain construction were performed with Q5 DNA polymerase. Thermocycling was performed using a PTC-200 thermocycler (MJ Research), except for the PCRs used to generate UBP-containing Golden Gate inserts and the PCRs used in the biotin shift assay, which were performed with a CFX-Connect Real-Time Thermal Cycler (Bio-Rad) to monitor product amplification with SYBR Green (Thermo Fisher). Where necessary, primers were phosphorylated using T4 polynucleotide kinase. Plasmids linearized by PCR were treated with DpnI to remove the plasmid template, and ligations were performed with T4 DNA ligase. PCRs and Golden Gate assembled plasmids were purified by spin column (DNA Clean and Concentrator-5, Zymo Research). DNA fragments isolated by agarose gel electrophoresis were purified using the Zymoclean Gel DNA recovery kit (Zymo Research). Colony PCRs were performed with Taq DNA polymerase. Natural DNA fragments and plasmids were quantified by A_{260/280} using a NanoDrop 2000 (Thermo Fisher) or an Infinite M200 Pro (Tecan). DNA fragments and plasmids that contain UBP(s), which were typically <20 ng/μL, were quantified using the Qubit dsDNA HS Assay Kit (Thermo Fisher).

The sequences of all DNA oligonucleotides used in this study are provided in Table 2. Natural oligonucleotides were purchased from IDT (San Diego, California, USA) with standard purification and desalting. Gene synthesis of the codon optimized PtNTT2 and GFP gene sequences was performed by GeneArt Gene Synthesis (Thermo Fisher) and GenScript, respectively, and kindly provided by Synthorx. Sequencing was performed by Eton Biosciences (San Diego, California, USA) or Genewiz (San Diego, California, USA). Plasmids were isolated using commercial miniprep kits (QIAprep, Qiagen or ZR Plasmid Miniprep Classic, Zymo Research).

[α−³²P]-dATP (3000 Ci/mmol, 10 mCi/mL) was purchased from PerkinElmer (Shelton, Connecticut, USA). Triphosphates of dNaM, d5SICS, dTPT3, and dMMO2^{bio} were synthesized as described in Li et al, “Natural-like replication of an unnatural base pair for the expansion of the genetic alphabet and biotechnology applications,” J. Am. Chem. Soc. 136, 825-829 (2014); or kindly provided by Synthorx (San Diego, California, USA). The dNaM-containing TK1 oligonucleotide was described in Malyshev, et al., “A semi-synthetic organism with an expanded genetic alphabet,” Nature, 509, 385-388 (2014). All other unnatural oligonucleotides containing dNaM were synthesized by Biosearch Technologies (Petaluma, California, USA) with purification by reverse phase cartridge.

The C₄₁(DE3) E. coli strain was kindly provided by J. P. Audia (University of South Alabama, USA). pKIKOarsBKm was a gift from Lars Nielsen & Claudia Vickers (Addgene plasmid #46766). pRS426 was kindly provided by Richard Kolodner (University of California San Diego, USA).

### Construction of PtNTT2 Plasmids

Construction of pCDF-1b-PtNTT2 was described Malyshev, et al., “A semi-synthetic organism with an expanded genetic alphabet,” Nature, 509, 385-388 (2014). To create pCDF-1b-PtNTT2(66-575), phosphorylated primers YZ552 and pCDF-1b-fwd were used to linearize pCDF-1b-PtNTT2 by PCR and the resulting product was intramolecularly ligated. Plasmids from single clones were isolated and confirmed by sequencing the PtNTT2 gene using primers T7 seq and T7 term seq.

To create plasmids pSC-P_{(lacI, bla, lac, lacUV5)}PtNTT2(66-575)-To, phosphorylated primers YZ581 and YZ576 were used to amplify the PtNTT2(66-575) gene, and its corresponding ribosomal binding sequence and terminator, from a version of pCDF-1b-PtNTT2(66-575) that replaces the T7 terminator with a λ T₀ terminator. This insert was ligated into plasmid pHSG576 linearized with primers DM002 and YZ580. A single clone of the resulting plasmid pSC-PtNTT2(66-575)-T₀ was verified by sequencing the PtNTT2 gene using primers DM052 and YZ50. pSC-PtNTT2(66-575)-T₀ was then linearized with primers YZ580 and YZ581, and ligated to a phosphorylated primer duplex corresponding to the P_{lacI}, P_{bla}, P_{lac} or P_{lacUV5} promoter (YZ584/YZ585, YZ582/YZ583, YZ599/YZ600, and YZ595/YZ596, respectively) to yield plasmids pSC-P_{(lacI, bla, lac, lacUV5)}PtNTT2(66-575)-To. Correct promoter orientation and promoter-gene sequences were again confirmed by sequencing using primers DM052 and YZ50. pSC-P_{bla}PtNTT2(66-575 co)-T₀ was generated analogously to pSC-P_{bla}PtNTT2(66-575)-T₀ by using a λ To terminator version of pCDF-1b-PtNTT2(66-575) containing a codon optimized PtNTT2 sequence (see Table 2).

### Construction of PtNTT2 and Cas9 Strains

The PtNTT2(66-575) expression cassette and its chloramphenicol resistance marker Cm^{R} in the pSC plasmids is ˜2.8 kb, a size that is prohibitive for chromosomal integration with the small (·50 bp) stretches of homology that can be introduced via primers during PCR, as is traditionally done in recombineering. Homologous recombination in S. cerevisiae was used to construct a series of integration template plasmids with the PtNTT2(66-575) expression cassette and Cm^{R} flanked by ˜1 kb of sequence 5′ to lacZ and ˜1 kb of sequence 3′ to 246 bp downstream of the lacA start codon. The lacZYA locus was chosen so that integration of the transporter would also knockout the lactose permease lacY, thus creating a BL21(DE3) strain that allows for uniform cellular entry of IPTG, and thereby homogenous, finely titratable induction of promoters containing lac operators.

To create the integration template plasmids, pRS426 was digested with PvuI-HF and the resulting 3810-bp plasmid fragment was isolated by agarose gel electrophoresis and purification. This fragment was then gap repaired in the S. cerevisiae strain BY4741 via lithium acetate mediated chemical transformation of the plasmid fragment and PCR products of the following primer/primer/template combinations: YZ7/YZ12/pBR322, YZ613/YZ580/E. coli genomic DNA, YZ614/615/E. coli genomic DNA, and DM052/YZ612/pSC-P_{lacUV5}(66-575)-T₀. The resulting plasmid, 426.lacZYA::P_{lacUV5}PtNTT2(66-575)-T₀ Cm^{R}, was isolated (Zymoprep Yeast Plasmid Miniprep, Zymo Research), digested with PvuI-HF and XbaI (to reduce background during integration, since the pRS426 shuttle plasmid also contains an E. coli pMB1 origin), and used as the template to generate a linear integration fragment via PCR with primers YZ616 and YZ617. Integration of this fragment into BL21(DE3) to generate strain YZ2 was performed using pKD46 as described in Datsenko, et al., “One-step inactivation of chromosomal genes in Escherichia coli K-12 using PCR products,” PNAS 97, 6640-6645 (2000). Integrants were confirmed by colony PCR of the 5′ and 3′ junctions using primers YZ618 and YZ587 (1601-bp product) and YZ69 and YZ619 (1402-bp product), respectively, detection of lacZ deletion via growth on plates containing X-gal (80 μg/mL) and IPTG (100 μM), and PCR and sequencing of the transporter with primers DM053 and YZ50.

Plasmid 426.IacZYA::Cm^{R} was generated from the linearization of 426.lacZYA::P_{lacUV5}PtNTT2(66-575)-T₀ Cm^{R} using phosphorylated primers YZ580 and pCDF-1b-rev, and subsequent intramolecular ligation. 426.IacZYA::Cm^{R} was then integrated into BL21(DE3) to create an isogenic, transporter-less control strain for dATP uptake assays.

To create plasmids 426.IacZYA::P_{(bla,lac,lacUV5)}PtNTT2(66-575 co)-T₀ Cm^{R}, plasmid 426.lacZYA::PtNTT2(66-575)-T₀ Cm^{R} (a promoter-less plasmid generated analogously to 426.lacZYA::P_{lacUV5}PtNTT2(66-575)-T₀ Cm^{R} using pSC-PtNTT2(66-575)-T₀), referred to hereafter as 426.trunc, was digested with PvuI-HF and AvrII, and the resulting 5969-bp plasmid fragment was isolated by agarose gel electrophoresis and purification, and gap repaired using PCR products of the following primer/primer/template combinations: 1, YZ12/YZ580/426.trunc; 2, DM053/YZ610/pSC-P_{(bla, lac, lacUV5)}PtNTT2(66-575)-T₀; 3, YZ581/YZ50/pSC-P_{bla}PtNTT2(66-575 co)-To. Plasmids 426.IacZYA::P_{(tac, N25, λ, H207)}PtNTT2(66-575 co)-T₀ Cm^{R} were generated analogously except fragments 2 were replaced with fragments corresponding to the promoters P_{tac}, P_{N25}, Pa and P_{H207}, which were generated by annealing and extension of primer pairs YZ703/YZ704, YZ707/YZ708, YZ709/YZ710, and YZ711/YZ712, respectively, with Klenow fragment. Plasmids 426.lacZYA::P_{(bla, lac, lacUV5, tac, N25, λ, H207)}PtNTT2(66-575 co)-T₀ Cm^{R} were then used to integrate the transporter into BL21(DE3) using primers YZ616 and YZ617, and recombineering, as described above. Strain YZ3 denotes BL21(DE3) integrated with IacZYA::P_{lacUV5}PtNTT2(66-575 co)-T₀ Cm^{R}.

To create strain YZ4, the 4362-bp fragment of SpeI and AvrII digested pCas9-Multi was ligated into SpeI digested pKIKOarsBKm and the resulting plasmid, pKIKOarsB::P_{lacO}-Cas9-TᵣᵣₙB Kₘ^{R}, was used as the template to generate a linear integration fragment via PCR with primers YZ720 and YZ721. The fragment was then integrated into BL21(DE3) as described above, and confirmed by colony PCR with primers YZ720 and YZ721 and sequencing of the product with primers TG1-TG6. P_{lacUV5}PtNTT2(66-575 co)-T₀ Cm^{R} was subsequently integrated into this strain, as described above, to generate strain YZ4.

dATP uptake assay

Radioactive uptake assays were conducted as described in Haferkamp, et al., “Tapping the nucleotide pool of the host: novel nucleotide carrier proteins of Protochlamydia amoebophila,” Mol. Microbiol. 60, 1534-1545 (2006) with the following modifications: C41(DE3) and BL21(DE3) strains carrying plasmid-based transporters and their appropriate empty plasmid controls, as well as BL21(DE3) chromosomal transporter integrants and their appropriate isogenic transporter-less control, were grown overnight with appropriate antibiotics (streptomycin for pCDF plasmids and chloramphenicol for pSC plasmids and integrants) in 500 μL of media. Cultures were diluted to an OD₆₀₀ of 0.02 in 500 μL of fresh media, grown for 2.5 h, induced with IPTG (0-1 mM, pCDF strains only) or grown (all other strains) for 1 h, and incubated with dATP spiked with [α−³²P]-dATP (final concentration=250 μM (0.5 μCi/mL)) for ˜1 h. This experimental scheme is analogous to the protocol used to prepare cells for transformation with UBP-containing plasmids, with the 1 h of dATP incubation simulating the 1 h of recovery in the presence of unnatural triphosphates following electroporation. A duplicate 48-well plate without[α−³²P]-dATP was grown in parallel to monitor growth.

Following incubation with dATP, 200 μL of each culture was collected through a 96-well 0.65 μm glass fiber filter plate (MultiScreen, EMD Millipore) under vacuum and washed with cold potassium phosphate (3×200 μL, 50 mM, pH 7) and cold ddH₂O (1×200 μL). Filters were removed from the plate and exposed overnight to a storage phosphor screen (BAS-IP MS, GE Healthcare Life Sciences), which was subsequently imaged using a flatbed laser scanner (Typhoon 9410, GE Healthcare Life Sciences). The resulting image was quantified by densitometric analysis using Image Studio Lite (LI-COR). Raw image intensities of each sample were normalized to the length of time and average OD₆₀₀ during dATP incubation (i.e. normalized to an estimate of the area under the growth curve corresponding to the window of uptake), followed by subtracting the normalized signals of the appropriate negative, no transporter controls.

Doubling times for strains grown in the dATP uptake assay were calculated by doubling time as (t₂-t₁)/log₂(OD_{600.2}/OD_{600.1}), averaging across three, ˜30 min time intervals roughly corresponding to 30 min prior to dATP uptake and 60 min during dATP uptake.

Construction of Golden Gate Destination Plasmids for pUCX1, pUCX2, and pBRX2

Although the UBP was cloned into plasmids via circular polymerase extension cloning (CPEC)^{4.27}, the method results in a doubly-nicked plasmid that cannot be treated with T5 exonuclease to degrade unincorporated linear plasmid and inserts, and thus makes it difficult to accurately quantify the yield of the cloning reaction and control the amount of input plasmid used to transform cells during an in vivo replication experiment. Furthermore, the unincorporated linear plasmid and inserts of a CPEC reaction can also template PCR reactions with the primers used in the biotin shift assay, and thus biotin shift assays on CPEC products do not truly reflect the UBP content of the plasmids that are actually transformed into cells. To circumvent these complications, the UBP was incorporated into plasmids using Golden Gate Assembly.

To create pUCX1 GG and pUCX2 GG, the Golden Gate destination plasmids for pUCX1 and pUCX2, respectively, pUC19 was linearized with phosphorylated primers pUC19-lin-fwd and pUC19-lin-rev, and the resulting product was intramolecularly ligated to delete the natural 75-nt TK1 sequence. The resulting plasmid was then linearized with phosphorylated primers YZ51 and YZ52, and the resulting product was intramolecularly ligated to mutate the BsaI recognition site within the ampicillin resistance marker Amp^{R}. This plasmid was then linearized with primers pUC19-lin-fwd and pUC19-lin-rev (for pUCX1), or primers YZ95 and YZ96 (for pUCX2), and ligated to an insert generated from PCR with phosphorylated primers YZ93 and YZ94 and template pCas9-Multi, to introduce two BsaI recognition sites (for cloning by Golden Gate Assembly) and a zeocin resistance marker (a stuffer cassette used to differentiate between plasmids with or without an insert) into pUC19.

To create pBRX2 GG, the Golden Gate destination plasmid for pBRX2, the 2934-bp fragment of AvaI and EcoRI-HF digested pBR322 was end-filled with Klenow fragment and intramolecularly ligated to delete the tetracycline resistance cassette. The BsaI recognition site within Amp^{R} was mutated as described above. The plasmid was then linearized with primers YZ95 and YZ96, and ligated to the BsaI-zeo^{R}-BsaI cassette as described above. Thus, pBRX2 is a lower copy analog of pUCX2.

Golden Gate Assembly of UBP-containing plasmids

Plasmids containing UBP(s) were generated by Golden Gate Assembly. Inserts containing the UBP were generated by PCR of chemically synthesized oligonucleotides containing dNaM, using dTPT3TP and dNaMTP, and primers that introduce terminal BsaI recognition sites that, when digested, produce overhangs compatible with an appropriate destination plasmid; see Table 2 for a full list of primers, templates and their corresponding Golden Gate destination plasmids. Template oligonucleotides (0.025 ng per 50 μL reaction) were PCR amplified using reagent concentrations and equipment under the following thermocycling conditions (times denoted as mm:ss): [96° C. 1:00|20× [96° C. 0:15|60° C. 0:15|68° C. 4:00]].

To assemble the UBP-containing plasmids, destination plasmid (200-400 ng), PCR insert(s) (3:1 insert:plasmid molar ratio), T4 DNA ligase (200 U), BsaI-HF (20 U), and ATP (1 mM) were combined in 1×NEB CutSmart buffer (final volume 30 μL) and thermocycled under the following conditions: [37° C. 20:00|40× [37° C. 5:00|16° C. 5:00|22° C. 2:30]37° C. 20:00|55° C. 15:00| 80° C. 30:00]. Following the Golden Gate reaction, T5 exonuclease (10 U) and additional BsaI-HF (20 U) were added, and the reaction was incubated (37° C., 1 h) to digest unincorporated plasmid and insert fragments. Assembled plasmids were quantified by Qubit.

Construction of Golden Gate destination plasmids for pCas9 and pAIO

To create pCas9-Multi, the Golden Gate destination plasmid for cloning sgRNA cassettes alongside Cas9, pPDAZ²⁹ and a PCR amplified Cas9 gene (Primers JL126 and JL128, template Addgene plasmid #41815) were digested with KpnI and XbaI, and ligated to create pCas9(−). This plasmid and a PCR amplified GFPT2-sgRNA cassette (template Addgene plasmid #41820, which contains the sgRNA sequence; the ProK promoter and terminator were introduced by PCR) were digested with SalI and ligated to created pCas9-GFPT2. This plasmid was then linearized with primers BL557 and BL558 (to remove the BsmBI recognition sites within Cas9) and circularized via Gibson Assembly. The resulting plasmid was then linearized with primers BL559 and BL560 (to reintroduce two BsmBI sites in the plasmid backbone), and circularized via Gibson Assembly to yield pCas9-Multi, which was confirmed by sequencing with primers TG1-TG6. Digestion of pCas9-Multi with BsmBI results in a linearized plasmid with overhangs that allow for the simultaneous cloning of one or more sgRNAs by Golden Gate Assembly (see section below).

To create pAIO-Multi, pCas9-Multi was linearized with primers BL731 and BL732 (to remove Cas9 and introduce BsaI recognition sites for UBP cloning), phosphorylated, and intramolecularly ligated, and confirmed by sequencing with primer BL450. Digestion of pAIO-Multi with BsaI results in a linearized plasmid with overhangs identical to the ones produced by BsaI digestion of the pUCX2 destination plasmid, and thus PCR-generated inserts for cloning the UBP into pUCX2 can also be used to clone the UBP into pAIO-Multi and its derivatives. After the sgRNA cassettes were cloned into pAIO-Multi (see next section below), the Golden Gate Assembly protocol for cloning in a UBP was identical to the one described above for pUCX2, except the product of pAIO-Multi (with sgRNAs) amplified with BL731 and BL732 was used in place of the plasmid itself.

sgRNA Cloning into pCas9 and pAIO

Dual sgRNA cassettes were cloned into pCas9-Multi or pAIO-Multi via Golden Gate Assembly. To generate the first sgRNA cassette of each pair, pCas9-GFPT2 (1 ng) was PCR amplified with primers 1′ sgRNA GG (200 nM) and BL562 (200 nM), and OneTaq DNA polymerase, under the following thermocycling conditions: [30× [94° C. 0:30|52° C. 0:15|68° C. 0:30]]. PCR products were purified by agarose gel electrophoresis and purification. The 1^{st} sgRNA GG primer is a 70-nt primer that possesses (from 5′ to 3′) a BsmBI restriction site, 10-nt of homology with the ProK promoter, an 18-nt variable guide (spacer) complementary to a UBP-mutation, and 25-nt of homology to the non-variable sgRNA scaffold. To generate the second sgRNA cassette, pCas9-GFPT2 was PCR amplified with primers BL563 and 2^{nd} sgRNA Rev, and primers BL566 and 2^{nd} sgRNA Fwd, and the resulting two products were combined and amplified by overlap extension PCR using primers BL563 and BL566, followed by agarose gel electrophoresis and purification.

To assemble the guide plasmids, pCas9-Multi (40 ng) or pAIO-Multi (20 ng), purified DNA of the first sgRNA cassette (4.5 ng) and second sgRNA cassette (8 ng), T4 DNA ligase (200 U), BsmBI (5 U), and ATP (1 mM) were combined in 1×NEB CutSmart reaction buffer (final volume 20 μL) and thermocycled under the following conditions: [5× [37° C. 6:00|16° C. 8:00]15×[55° C. 6:00| 16° C. 8:00]]. Assembled plasmids were transformed into electrocompetent cells for subsequent sequencing and testing.

To assemble pCas9-TK1-A, a plasmid containing only one sgRNA cassette, pCas9-GFPT2 was amplified with primers BL566 and BL567, and the resulting product was ligated into pCas9-Multi by Golden Gate Assembly as described above.

To assemble pCas9-hEGFP, a plasmid containing a non-target sgRNA cassette for TK1 experiments, primers BL514 and BL515 were annealed and ligated, by Gibson Assembly, into pCas9-GFPT2 linearized with primers BL464 and BL465.

Construction of pAIO2X

pAIO2X GG, the Golden Gate destination plasmid for pAIO2X, is derived from three plasmids, using PCR-generated inserts and multiple steps of cloning by restriction enzyme digest and ligation. Inserts from pSYN36, which contains a codon-optimized superfolder gfp with a Golden Gate entry site for cloning in sequences that correspond to nucleotides 409-483 of gfp, and pET-22b-ESerGG, which contains an E. coli serT gene with a Golden Gate entry site for cloning in sequences that correspond to nucleotides 10-65 of serT, were cloned into pAIO dual guide BsmBI, a version of pAIO-Multi that contains two sgRNA cassettes, with the targeting guide (spacer) sequences replaced by two orthogonal pairs of BsmBI recognition sites that enable guide cloning using annealed primer duplexes.

To create pAIO2X-GFP151/Eser-69 GG, annealed primer duplexes of YZ310/YZ316 and YZ359/YZ360 were ligated into pAIO2X GG using the same Golden Gate Assembly reagents and thermocycling conditions used for UBP cloning, with the exception that BsaI was replaced by BsmBI, each primer duplex was used at a 50:1 insert:plasmid molar ratio with 30 fmol of destination plasmid, and the reaction was scaled by one third to 10 μL. Following assembly, the reaction was not digested with additional enzymes or purified, and was directly transformed into chemically competent E. coli DH5a. Following isolation of single plasmid clones and confirmation of the guides by sequencing using primer BL450, the UBPs were cloned into the plasmid by Golden Gate assembly with BsaI, as described in the section, Golden Gate Assembly of UBP-containing plasmids.

### Cas9 In Vitro Cleavage Assay

To generate the DNA substrates for in vitro Cas9 cleavage assays, templates BL408, BL409, BL410, BL487, BL488, and BL489 (1 ng per 50 μL reaction) were PCR amplified with primers BL415 (400 nM) and BL416 (400 nM), and OneTaq DNA polymerase in 1×OneTaq standard reaction buffer supplemented with dNaMTP (100 μM), dTPT3TP (100 μM), and MgCl₂ (1.5 mM), under the following thermocycling conditions: [25× [95° C. 0:15|56° C. 0:15| 68° C. 1:30]].

To generate the DNA templates for in vitro transcription of sgRNAs, templates BL318, BL484, BL485, and BL486 (1 ng per 50 μL reaction), which contain the T7 promoter and a CRISPR RNA (crRNA) spacer sequence, were PCR amplified with primers BL472 (200 nM) and BL473 (200 nM), and OneTaq DNA polymerase in 1×OneTaq standard reaction buffer supplemented with MgCl₂ (6 mM), under the following thermocycling conditions: [20×[95° C. 0:15| 60° C. 0:15| 68° C. 1:30]]. DNA from this first PCR reaction (0.5 μL) was then transferred into a second PCR reaction (100 μL) containing primers BL472 (400 nM), BL439 (500 nM), and BL440 (600 nM), and thermocycled under the following conditions: [4×[95° C. 0:15|68° C. 0:15| 68° C. 1:30]20×[95° C. 0:15| 60° C. 0:15| 68° C. 1:30]]. In vitro transcription of the PCR products with T7 RNA polymerase was performed, and transcribed sgRNAs were purified by PAGE, band excision, and extraction (37° C., overnight) into an aqueous solution of NaCl (200 mM) and EDTA (1 mM, pH 7), followed by concentration and purification by ethanol precipitation.

For in vitro cleavage reactions, Cas9 nuclease (125 nM) was incubated with each transcribed sgRNA (125 nM) in 1×Cas9 nuclease reaction buffer for 5 min, then DNA substrate was added and the reaction was incubated (37° C., 10 min). The reaction was quenched with SDS-PAGE loading buffer (62 mM Tris-HCl, 2.5% SDS, 0.002% bromophenol blue, 0.7 M β-mercaptoethanol, and 10% glycerol), heat denatured (95° C., 10 min), and then loaded onto an SDS-PAGE gel. The resulting cleavage bands were quantified by densitometric analysis using ImageJ³¹. For each sgRNA, raw cleavage efficiencies were divided by the maximum cleavage observed for that sgRNA across the set of the six DNA substrates, to account for differences in sgRNA activity and/or minor variations in preparation. Experiments were performed in technical triplicate and averages represent an average of three in vitro cleavage reactions performed in parallel.

### In Vivo Plasmid Replication Experiments

Electrocompetent YZ3 cells were prepared by overnight growth in ˜5 mL of media supplemented with chloramphenicol, dilution to OD₆₀₀ of 0.02 in the same media (variable volumes, ˜10 mL of media per transformation), and growth to OD₆₀₀ of ˜0.3-0.4. Cells were then rapidly chilled in an ice water bath with shaking, pelleted (2500×g, 10 min), and washed twice with one culture volume of ice-cold ddH₂O. Electrocompetent cells were then resuspended in ice-cold ddH₂O (50 μL per transformation), mixed with a Golden Gate assembled plasmid (˜1 μL, ˜1 ng) containing the UBP, and transferred to a pre-chilled 0.2 cm gap electroporation cuvette. Cells were electroporated (Gene Pulser II, Bio-Rad) according to the manufacturer's recommendations (voltage 25 kV, capacitor 2.5 μF, resistor 200Ω) then immediately diluted with 950 μL of pre-warmed media supplemented with chloramphenicol. An aliquot (10-40 μL) of this dilution was then immediately diluted 5-fold with the same pre-warmed media, but additionally supplemented with dNaMTP (250 μM) and d5SICSTP (250 μM). The samples were incubated (37° C., 1 h) and then ˜15% of the sample was used to inoculate media (final volume 250-300 μL) supplemented with chloramphenicol, carbenicillin, dNaMTP (250 μM) and d5SICSTP (250 μM). Cells were then monitored for growth, collected at the density (OD₆₀₀) indicated in the main text, and subjected to plasmid isolation. Dilutions of the recovery mixture were also spread onto solid media with chloramphenicol and carbenicillin to ascertain transformation efficiencies. Experiments with dNaMTP (150 μM) and dTPT3TP (37.5 μM) were performed analogously.

Experiments with DM1 were performed analogously using media supplemented with streptomycin, with the additional step of inducing transporter expression with IPTG (1 mM, 1 h) prior to pelleting the cells. All media following electrocompetent cell preparation was also supplemented with streptomycin and IPTG (1 mM) to maintain expression of the transporter.

In Vivo Plasmid Replication Experiments with Cas9 (Liquid Culture Only)

Electrocompetent YZ2 cells were transformed with various pCas9 guide plasmids and single clones were used to inoculate overnight cultures. Cells were then grown, prepared and electroporated as described above for YZ3, with the following modifications: all media was additionally supplemented with zeocin (to select for pCas9) and 0.2% glucose, electrocompetent cells were stored in 10% (v/v ddH₂O) DMSO at −80° C. until use, and recovery and growth media were supplemented with dNaMTP (250 μM) and dTPT3TP (75 μM). Varying concentrations of IPTG (0-100 μM) were added to the growth media (but not the recovery media) to induce Cas9 expression. The sgRNAs corresponding to the d(AXT) sequence are the non-target guides for all sequences except for the d(AXT)-containing sequence itself, the non-target guides for which correspond to the d(GXT) sequence and all experiments with non-target sgRNAs were conducted with the addition of IPTG (10 μM) to the growth media. For growth and regrowth experiments, cells were grown to an OD₆₀₀ of 3.5-4.0, then diluted 1:250 and regrown to an OD₆₀₀ of 3.5-4.0, after which plasmids were isolated.

In Vivo Plasmid Replication Experiments with Cas9 (Plating and Liquid Culture)

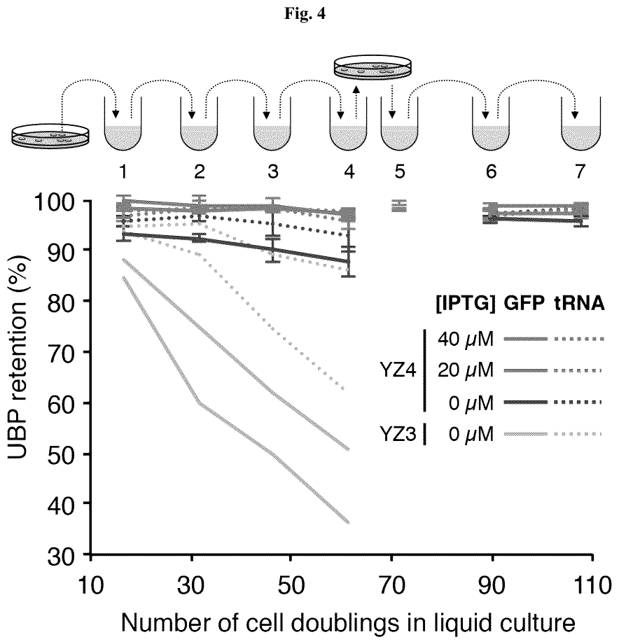

Electrocompetent YZ4 cells were grown, prepared and electroporated as described above for YZ2, with the following modifications: media for growing cells prior to electroporation only contained chloramphenicol (i.e. no zeocin), zeocin was used to select for pAIO (i.e. no carbenicillin), and recovery and growth media were supplemented with dNaMTP (150 μM) and dTPT3TP (37.5 μM). Following transformation with pAIO, dilutions of the recovery mixture were spread onto solid media containing chloramphenicol, zeocin, dNaMTP (150 μM), dTPT3TP (37.5 μM), 0.2% glucose, and various concentrations of IPTG (0-50 μM). Following overnight growth (37° C., ˜14 h), individual colonies were used to inoculate liquid media of the same composition as the solid media. Experiments performed with pAIO2X were conducted as described above for YZ4 without using frozen electrocompetent cells or glucose. The second plating depicted in FIG. 4 was performed by streaking cells from liquid culture onto solid media of the same composition as the liquid media, and growth at 37° C. (˜14 h). Six random colonies were selected to continue propagation in liquid culture.

### Cell Doubling Calculation

Cell doublings for liquid culture growth-dilution-regrowth experiments were calculated by log₂ of the dilution factor (30,000 or 300,000) between growths, except for growths inoculated from a plated colony, the cell doublings for which were calculated by averaging, for each individual clone, the time from inoculation to target OD₆₀₀ (9.4±1.1 h (1 SD) for the first plating inoculation, 10.2±3 h for the second plating inoculation) and dividing these averages by an estimated doubling time of 40 min. Growth times varied for each clone because colonies were isolated when they were barely visible to the naked eye, and thus it was not attempted to control for variability in the number of cells inoculated into the liquid cultures. Note that the reported cell doublings was only an estimate of doublings in liquid culture, which underreported the total number of cell doublings, as it was not attempted to estimate the number of cell doublings that occurred during each of the growths on solid media.

### Biotin Shift Assay

The retention of the UBP(s) in isolated plasmids was determined and validated as follows: plasmid minipreps or Golden Gate assembled plasmids (0.5*L, 0.5-5 ng/μL), or dNaM-containing oligonucleotides (0.5 fmol), were PCR amplified with dNTPs (400 μM), 1× SYBR Green, MgSO₄ (2.2 mM), primers (10 nM each), d5SICSTP (65 μM), dMMO2^{Bio}TP (65 μM), OneTaq DNA polymerase (0.018 U/μL), and DeepVent DNA polymerase (0.007 U/μL) in 1× OneTaq standard reaction buffer (final volume 15 μL), under the following thermocycling conditions: [20× [95° C. 0:15 |×° C. 0:15| 68° C. 4:00]]; see Table 2 for a list of primers and their corresponding annealing temperatures used in this assay. After amplification, 1 μL of each reaction was mixed with streptavidin (2.5 μL, 2 μg/μL, Promega) and briefly incubated at 37° C. After incubation, samples were mixed with loading buffer and run on a 6% polyacrylamide (29:1 acrylamide:bis-acrylamide) TBE gel, at 120 V for ˜30 min. Gels were then stained with 1×SYBR Gold dye (Thermo Fisher) and imaged using a Molecular Imager Gel Doc XR+(Bio-Rad) equipped with a 520DF30 filter (Bio-Rad).

### Calculation of UBP Retention

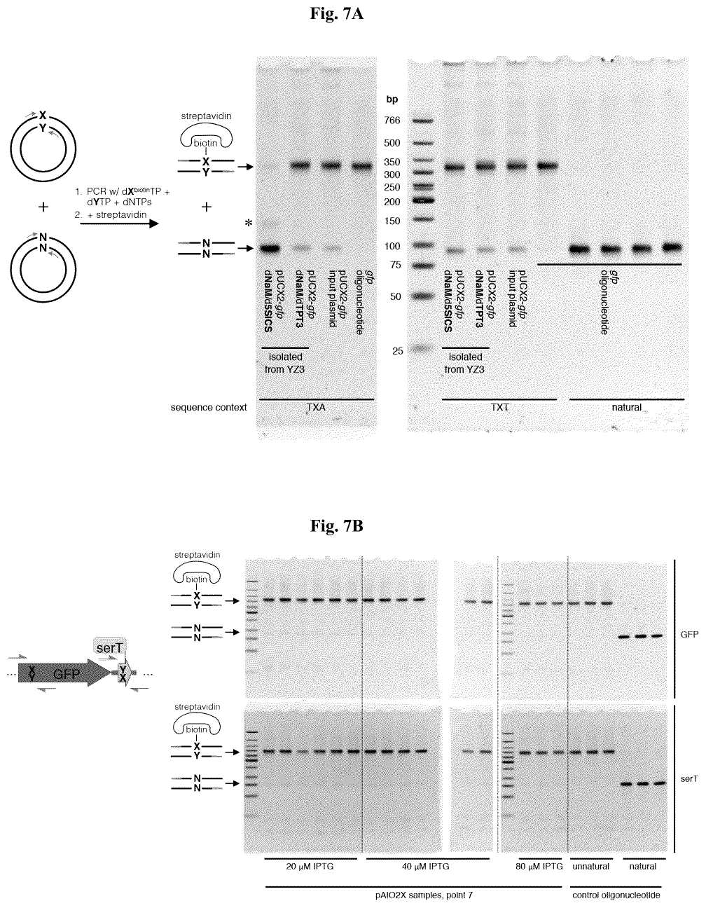

UBP retention was assessed by densitometric analysis of the gels (ImageJ or Image Studio Lite, LICOR) from the biotin shift assay and calculation of a percent raw shift, which equals the intensity of the streptavidin-shifted band divided by the sum of the intensities of the shifted and unshifted bands. See FIG. 7 for representative gels. Reported UBP retentions are normalized values.

Unless otherwise indicated, for experiments not involving plating on solid media, UBP retention was normalized by dividing the percent raw shift of each propagated plasmid sample by the percent raw shift of the Golden Gate assembled input plasmid. It was assumed that the starting UBP content of the cellular plasmid population was equivalent to the UBP content of the input plasmid, based on direct inoculation of the transformation into liquid culture. Thus, in these experiments, normalized UBP retention was a relative value that related the UBP content of the propagated plasmid population to the UBP content of the starting population, which was not 100% due to loss during the PCR used to generate the insert for input plasmid assembly (FIG. 7).

For experiments involving plating on solid media, UBP retention was normalized by dividing the percent raw shift of each propagated plasmid sample by the percent raw shift of the dNaM-containing oligonucleotide template used in the assembly of the input plasmid. Plating enabled clonal isolation of UBP-containing plasmids from fully natural plasmids that arose during plasmid construction (some of which may contain sequences that were not recognized by the sgRNA(s) employed). Because there was no PCR-mediated loss of the UBP in the oligonucleotide template, normalization to the oligonucleotide template was a better indicator of absolute UBP retention than normalization to the input plasmid. Under the conditions used in the biotin shift assay, most oligonucleotide templates and sequence contexts gave >90% raw shift, with <2% shift for a cognate fully natural template (i.e. UBP misincorporation during the biotin shift assay was negligible).

Plating allowed for the differentiation between UBP loss that occurred in vivo from loss that occurred in vitro, with the exception of clonally-derived samples that gave <2% shift, for which it was unable to differentiate between whether the UBP was completely lost in vivo or if the sample came from a transformant that originally received a fully natural plasmid. Such samples were excluded from reported average values when other samples from the same transformation give higher shifts.

### Biotin Shift Depletion and In Vivo Mutation Analysis

To determine the mutational spectrum of the UBP in isolated plasmid samples, biotin shift assays were performed as described above. Non-shifted bands, which corresponded to natural mutations of the UBP-containing sequences, were excised and extracted (37° C., overnight) into a minimal amount of an aqueous solution of NaCl (200 mM) and EDTA (1 mM, pH 7), followed by concentration and purification by ethanol precipitation. A sample of extract (1 μL) was PCR amplified under standard conditions (natural dNTPs only), with OneTaq DNA polymerase and the same primers used for the biotin shift PCR, and the resulting products were sequenced by Sanger sequencing.

### Functional Characterization of a Mutant PtNTT2 Transporter

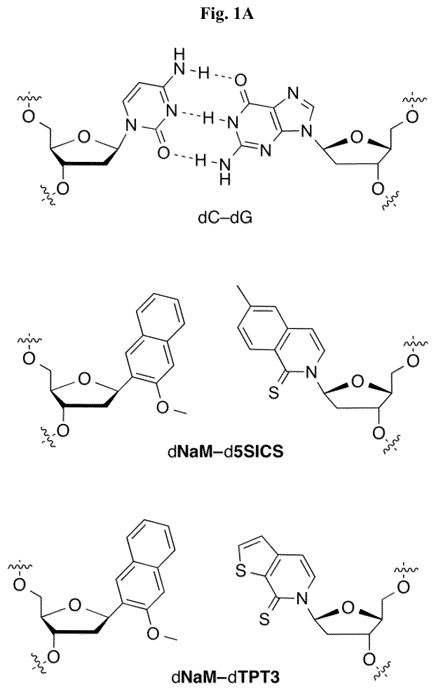

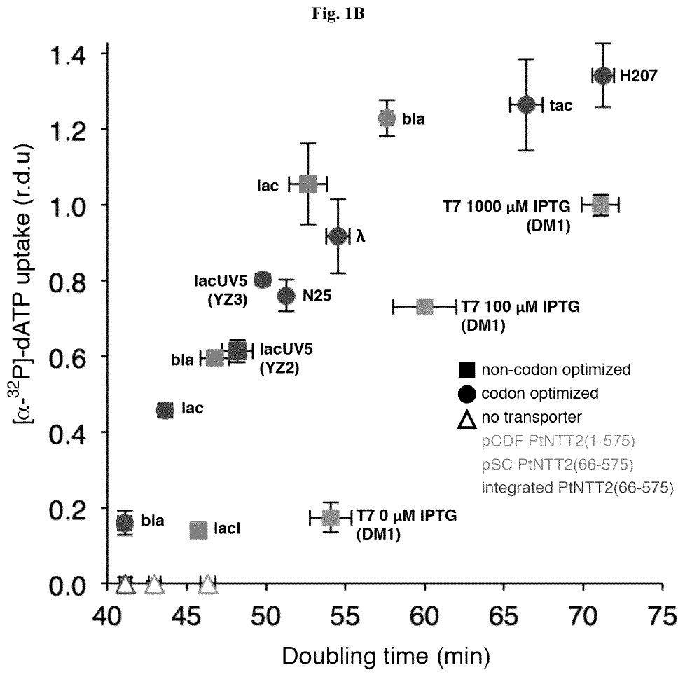

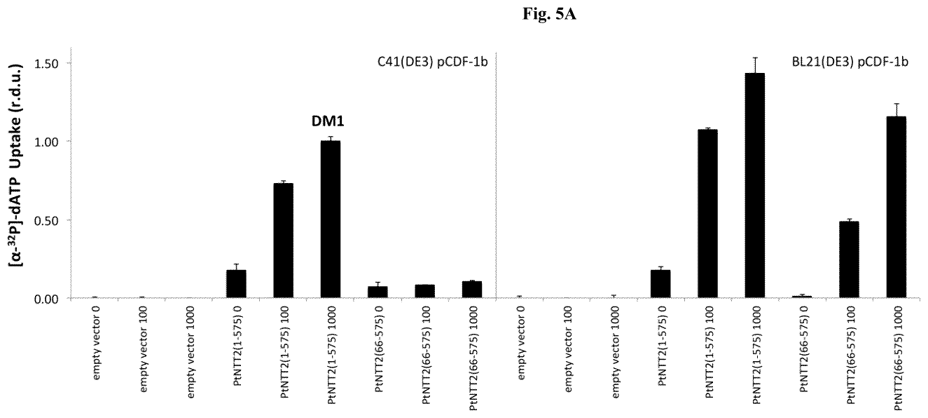

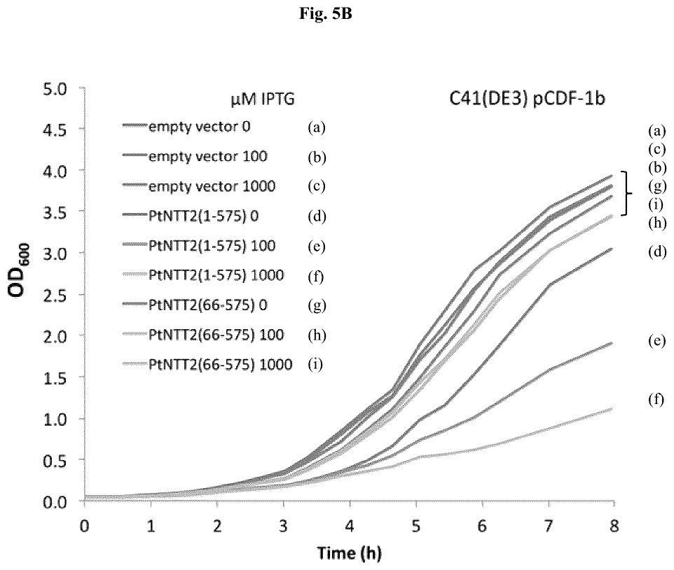

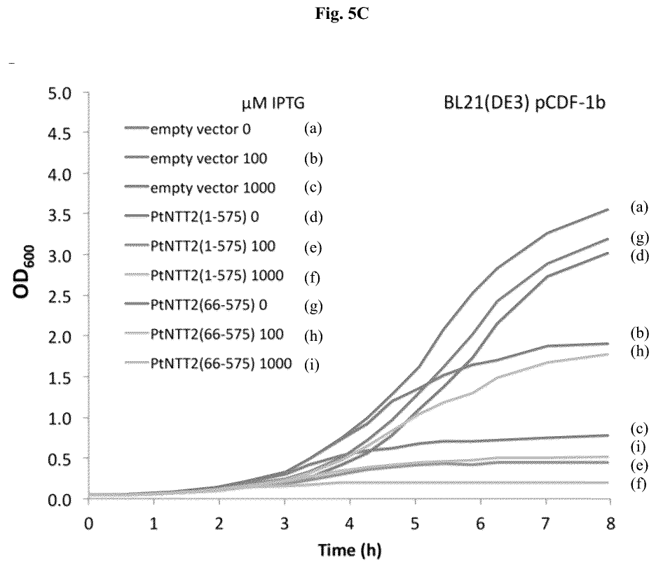

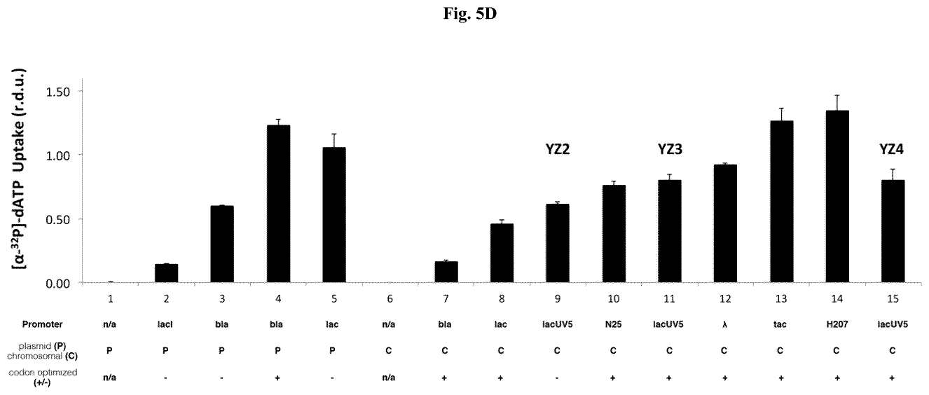

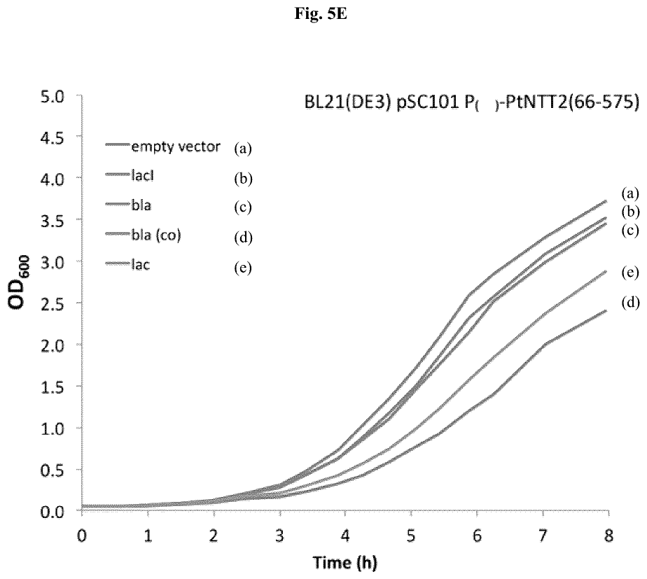

Expression of the nucleoside triphosphate transporter from Phaeodactylum tricornutum (PtNTT2) in E. coli enabled the import of dNaMTP and d5SICSTP and the subsequent replication of the dNaM-d5SICS UBP (FIG. 1A), but its expression was also toxic (FIG. 1). In SSO referred to herein as DM1, the transporter was expressed from a T7 promoter on a multicopy plasmid (pCDF-1b) in E. coli C41(DE3), and its induction was controlled due to the associated toxicity. In its native algal cell, PtNTT2's N-terminal signal sequences direct its subcellular localization and are removed by proteolysis. In some cases in the E. coli system, the N-terminal signal was retained, and contributed to the observed toxicity. Removal of amino acids 1-65 and expression of the resulting N-terminally truncated variant PtNTT2(66-575) in E. coli C41(DE3) resulted in lower toxicity relative to the full length PtNTT2, but also reduced uptake of [α−³²P]-dATP (FIG. 5A and FIG. 5B), possibly due to reduced expression. Expression of PtNTT2(66-575) in E. coli BL21(DE3) resulted in increased levels of [α−³²P]-dATP uptake with little increase in toxicity relative to an empty vector control (FIG. 5A and FIG. 5C), but the higher level of T7 RNAP in this strain was itself toxic (FIG. 5A and FIG. 5C).

Constitutive expression of PtNTT2(66-575) from a low copy plasmid or a chromosomal locus was explored, to eliminate the need to produce toxic levels of T7 RNAP, and to impart the SSO with greater autonomy, more homogeneous transporter expression and triphosphate uptake across a population of cells, and ultimately improve UBP retention. Expression of PtNTT2(66-575) in E. coli BL21(DE3) was explored with the E. coli promoters P_{lacI}, P_{bla}, and P_{lac} from a pSC plasmid, and with P_{bla}, P_{lac}, P_{lacUV5}, P_{H207}, Pλ, P_{tac}, and P_{N25} from the chromosomal lacZYA locus (see Table 2). The use of a codon-optimized variant of the truncated transporter was also explored (see Table 2). Although uptake of [α−³²P]-dATP was negatively correlated with doubling time, each strain exhibited an improved ratio of uptake to fitness compared to DM1 (FIG. 1). Strain YZ3, which expressed the codon-optimized, chromosomally integrated PtNTT2(66-575) from the P_{lacUV5} promoter, exhibited both robust growth (<20% increased doubling time relative to the isogenic strain without the transporter), and reasonable levels of [α−³²P]-dATP uptake, and was selected for further characterization.

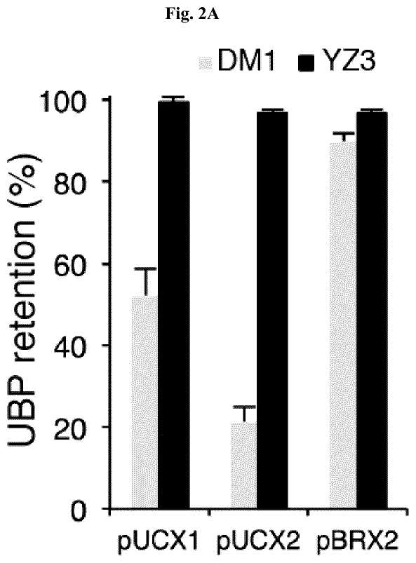

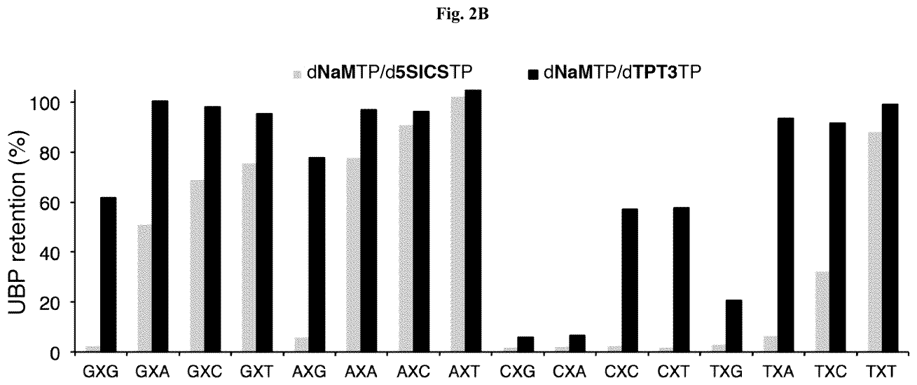

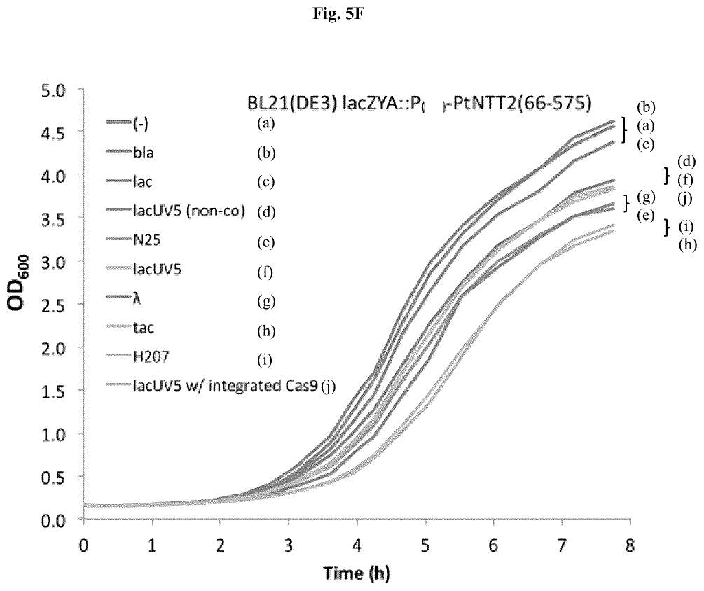

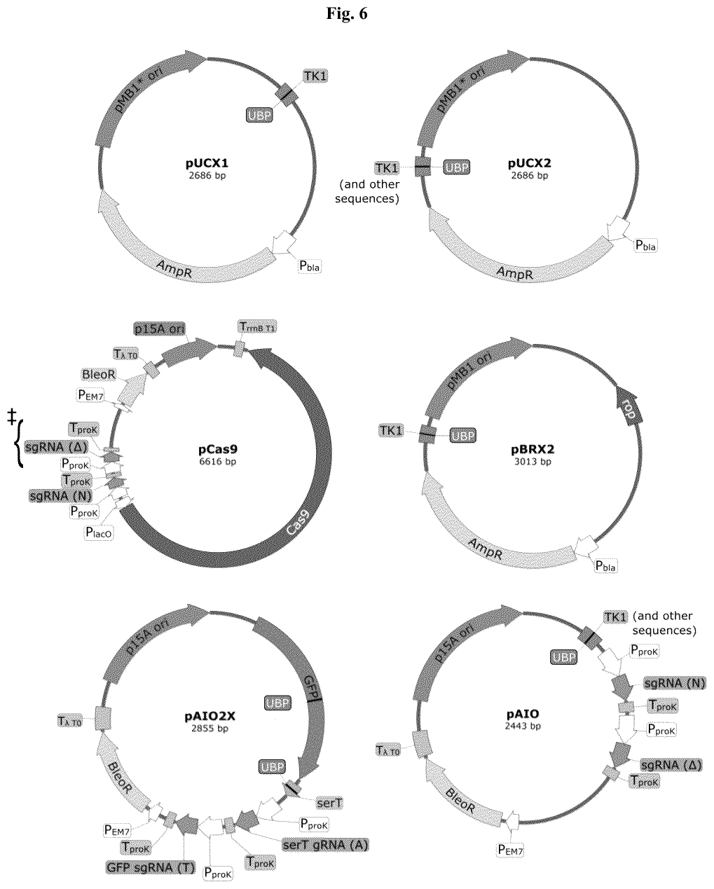

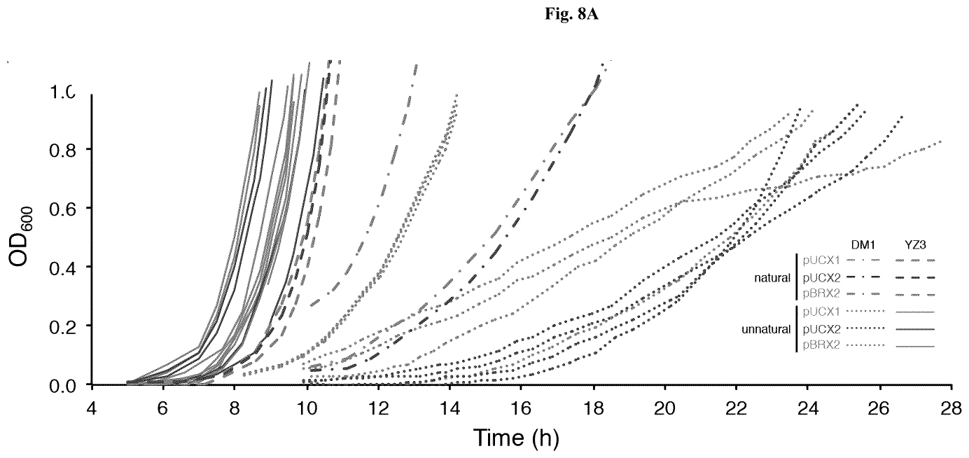

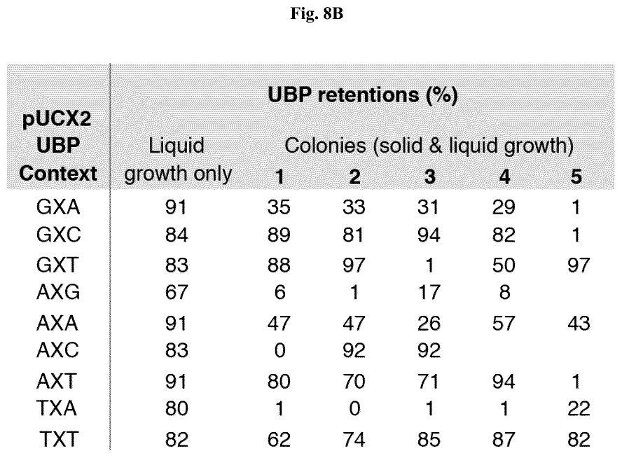

To determine whether the optimized transporter system of YZ3 facilitates high UBP retention, three plasmids that position the UBP within the 75-nt TK1 sequence were constructed (with a local sequence context of d(A-NaM-T)). These include two high copy pUC19-derived plasmids, pUCX1 and pUCX2, as well as one low copy pBR322-derived plasmid, pBRX2 (FIG. 6). In addition to examine the effect of copy number on UBP retention, these plasmids positioned the UBP at proximal (pUCX1) and distal (pUCX2 and pBRX2) positions relative to the origin of replication. E. coli YZ3 and DM1 were transformed with pUCX1, pUCX2, or pBRX2 and directly cultured in liquid growth media supplemented with dNaMTP and d5SICSTP (and IPTG for DM1 to induce the transporter), and growth and UBP retention were characterized (at an OD₆₀₀ of ˜1) (see Methods and FIG. 7A). While DM1 showed variable levels of retention and reduced growth with the high copy plasmids, YZ3 showed uniformly high levels of UBP retention and robust growth (FIG. 2a and FIG. 8A).

To explore the effect of local sequence context on UBP retention in YZ3, sixteen pUCX2 variants were constructed in which the UBP was flanked by each possible combination of natural base pairs within a fragment of gfp (see Table 2). Under the same growth conditions as above, a wide range of UBP retentions was observed, with some sequence contexts showing complete loss of the UBP (FIG. 2B). However, since the development of DM1 with the dNaM-d5SICS UBP, it was determined that ring contraction and sulfur derivatization of d5SICS, yielding the dNaM-dTPT3 UBP (FIG. 1A), resulted in more efficient replication in vitro. To explore the in vivo use of dNaM-dTPT3, the experiments were repeated with YZ3 and each of the sixteen pUCX2 plasmids but with growth in media supplemented with dNaMTP and dTPT3TP. UBP retentions were clearly higher with dNaM-dTPT3 than with dNaM-d5SICS (FIG. 2B).

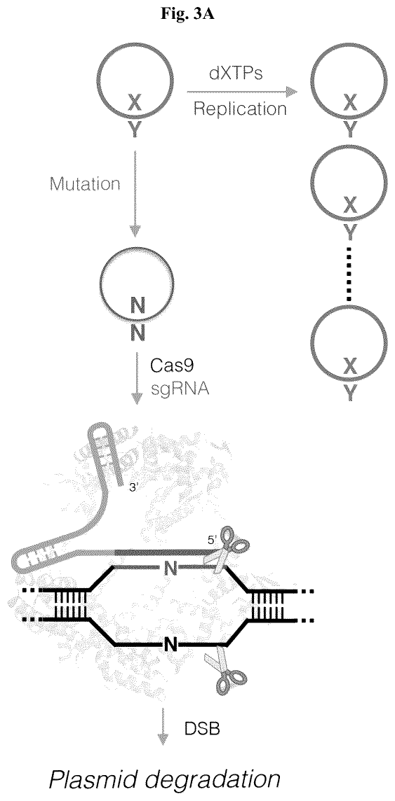

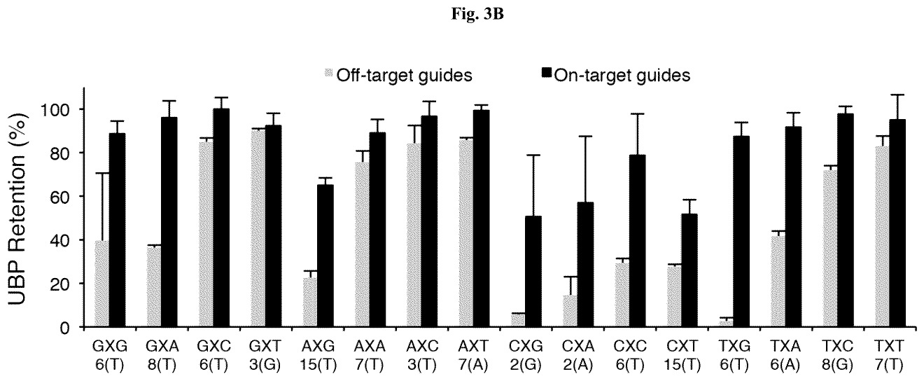

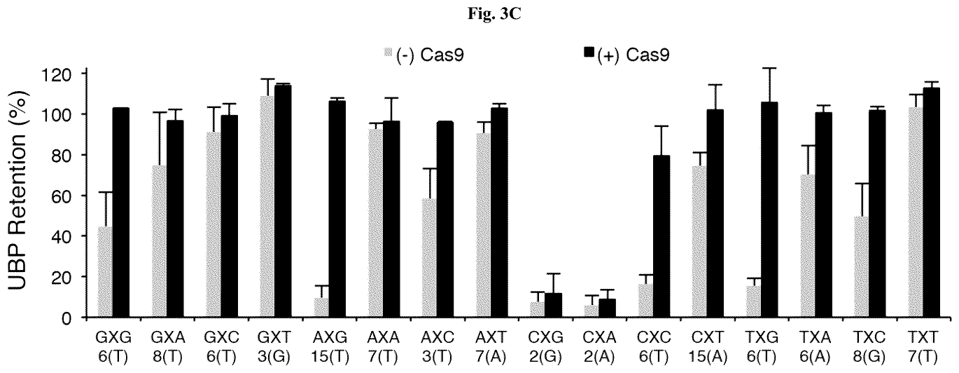

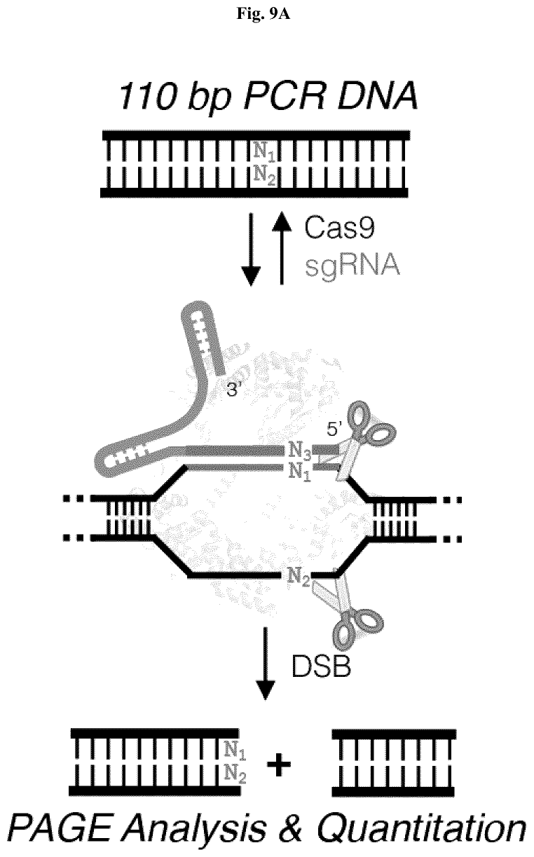

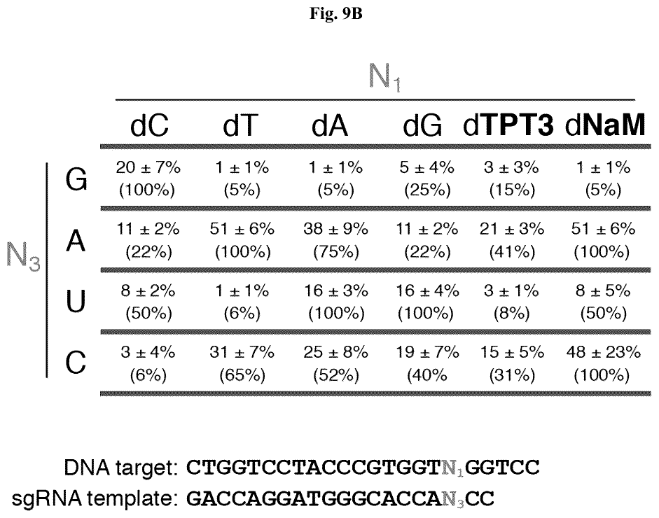

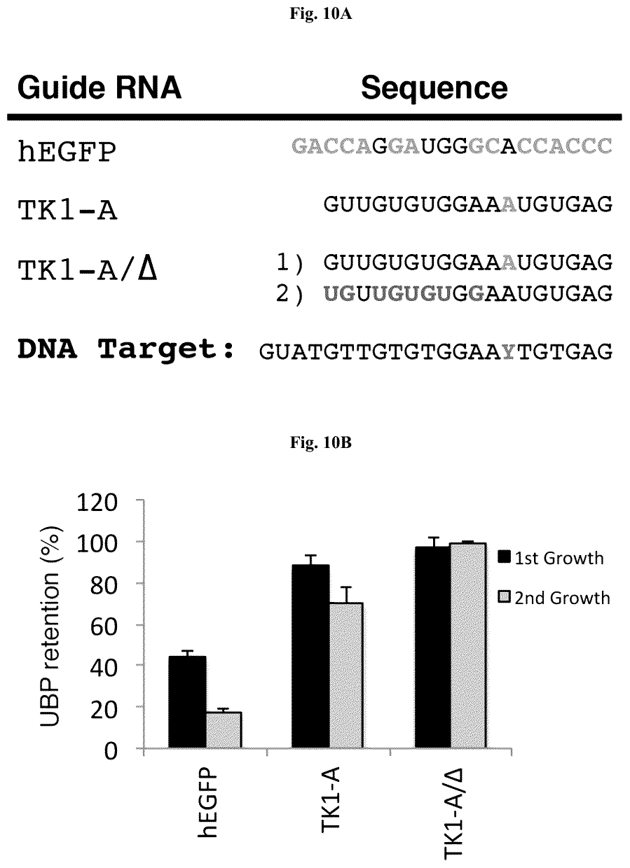

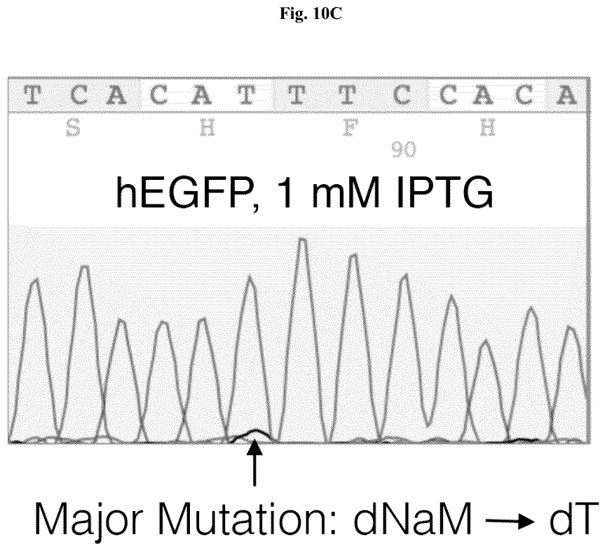

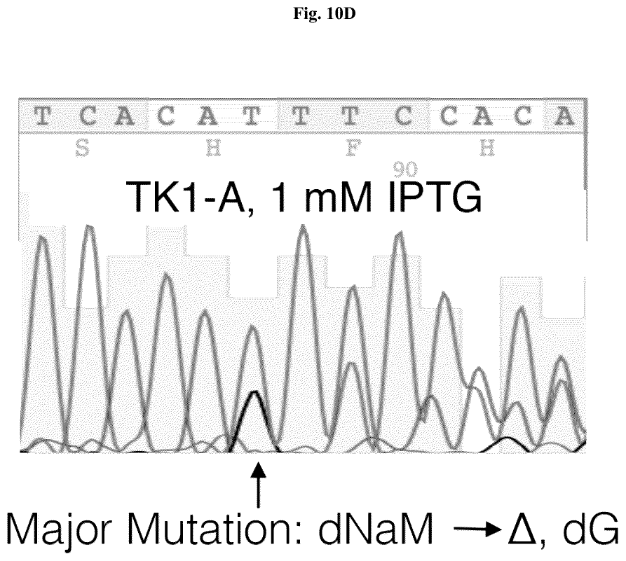

While dNaM-dTPT3 is a more optimal UBP for the SSO than dNaM-d5SICS, its retention is still moderate to poor in some sequences. Moreover, several sequences that show good retention in YZ3 cultured in liquid media show poor retention when growth includes culturing on solid media (FIG. 8B). To further increase UBP retention with even these challenging sequences and/or growth conditions, selective elimination of plasmids was carried out that lose the UBP. In prokaryotes, the clustered regularly interspaced short palindromic repeats (CRISPR)/CRISPR-associated (Cas) system provides adaptive immunity against viruses and plasmids. In type II CRISPR-Cas systems, such as that from Streptococcus pyogenes, the endonuclease Cas9 utilizes encoded RNAs (or their artificial mimics known as single-guide RNAs (sgRNAs)) to introduce double-strand breaks into complementary DNA upstream of a 5′-NGG-3′ protospacer adjacent motif (PAM) (FIG. 3A), which then results in DNA degradation by exonucleases. In vitro, it was found that the presence of a UBP in the target DNA generally reduces Cas9-mediated cleavage relative to sequences that are fully complementary to the provided sgRNA (FIG. 9). In some instances within a cell, Cas9 programmed with sgRNA(s) complementary to natural sequences that arise from UBP loss would enforce retention in a population of plasmids, which was refer to as immunity to UBP loss. To test this, a p15A plasmid was used to construct pCas9, which expresses Cas9 via an IPTG-inducible LacO promoter, as well as an 18-nt sgRNA that is complementary to the TK1 sequence containing the most common dNaM-dTPT3 mutation (dT-dA) via the constitutive ProK promoter (FIGS. 6 and 10A). Strain YZ2 (a forerunner of YZ3 with slightly less optimal transporter performance; FIG. 1A, FIG. 5D, and FIG. 5F) carrying the pCas9 plasmid was transformed with the corresponding pUCX2 plasmid (i.e. the pUCX2 variant with the UBP embedded within the TK1 sequence such that loss of the UBP produces a sequence targeted by the sgRNA encoded on pCas9), grown to an OD₆₀₀ of ˜4, diluted 250-fold, and regrown to the same OD₆₀₀. UBP retention in control experiments with a non-target sgRNA dropped to 17% after the second outgrowth; in contrast UBP retention in the presence of the correct sgRNA was 70% (FIG. 10B). Sequencing revealed that the majority of plasmids lacking a UBP when the correct sgRNA was provided contained a single nucleotide deletion in its place, which was not observed with the non-target sgRNA (FIG. 10C, and FIG. 10D). With a pCas9 plasmid that expresses two sgRNAs, one targeting the most common substitution mutation and one targeting the single nucleotide deletion mutation (FIG. 6), and the same growth and regrowth assay, loss of the UBP was undetectable (FIG. 10B).

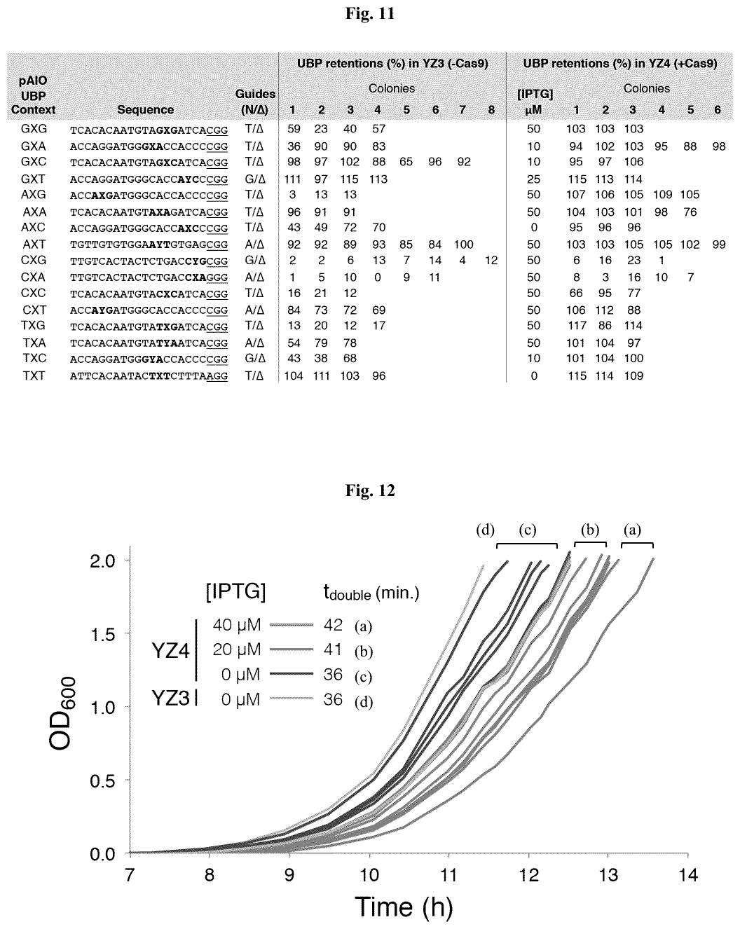

To more broadly explore Cas9-mediated immunity to UBP loss, retention was examined using sixteen pUCX2 variants with sequences that flank the UBP with each possible combination of natural base pairs, but also vary its position relative to the PAM, and vary which unnatural nucleotide is present in the strand recognized by the sgRNAs (FIG. 11). A corresponding set of sixteen pCas9 plasmids was also constructed that express two sgRNAs, one targeting a substitution mutation and one targeting the single nucleotide deletion mutation, for each pUCX2 variant. Strain YZ2 carrying a pCas9 plasmid was transformed with its corresponding pUCX2 variant and grown in the presence of the unnatural triphosphates and IPTG (to induce Cas9), and UBP retention was assessed after cells reached an OD₆₀₀ of ˜1. As a control, the sixteen pUCX2 plasmids were also propagated in YZ2 carrying a pCas9 plasmid with a non-target sgRNA. For four of the sixteen sequences explored, UBP loss was already minimal without immunity (non-target sgRNA), but was undetectable with expression of the correct sgRNA (FIG. 3B). The remaining sequences showed moderate to no retention without immunity, and significantly higher retention with it, including at positions up to 15 nts from the PAM.

To further simplify and streamline the SSO, strain YZ4 was constructed by integrating an IPTG-inducible Cas9 gene at the arsB locus of the YZ3 chromosome, which allows for the use of a single plasmid that both carries a UBP and expresses the sgRNAs that enforce its retention. Sixteen such “all in one” plasmids (pAIO) were constructed by replacing the Cas9 gene in each of the pCas9 variants with a UBP sequence from the corresponding pUCX2 variant (Extended Data FIG. 2). YZ4 and YZ3 (included as a no Cas9 control due to leaky expression of Cas9 in YZ4) were transformed with a single pAIO plasmid and cultured on solid growth media supplemented with the unnatural triphosphates and with or without IPTG to induce Cas9. Single colonies were used to inoculate liquid media of the same composition, and UBP retention was assessed after cells reached an OD₆₀₀ of ˜1-2 (FIG. 3C). Despite variable levels of retention in the absence of Cas9 (YZ3), with induction of Cas9 expression in YZ4, loss was minimal to undetectable in 13 of the 16 the sequences. While retention with the three problematic sequences, d(C-NaM-C) d(C-NaM-A) and d(C-NaM-G), might be optimized, for example, through alterations in Cas9 or sgRNA expression, the undetectable loss of the UBP with the majority of the sequences after a regimen that included growth both on solid and in liquid media, which was not possible with our previous SSO DM1, attests to the vitality of YZ4.

Finally, a pAIO plasmid, pAIO2X, was constructed containing two UBPs: dNaM paired opposite dTPT3 at position 451 of the sense strand of the gfp gene and dTPT3 paired opposite dNaM at position 35 of the sense strand of the serT tRNA gene, as well as encoding the sgRNAs targeting the most common substitution mutation expected in each sequence (FIG. 6). YZ4 and YZ3 were transformed with pAIO2X and subjected to the challenging growth regime depicted in FIG. 4, which included extensive high-density growth in liquid and on solid growth media. Plasmids were recovered and analyzed for UBP retention (FIG. 7B) when the OD₆₀₀ reached 1-2 during each liquid outgrowth. In YZ3, which does not express Cas9, or in the absence of Cas9 induction (no IPTG) in YZ4, UBP retention steadily declined with extended growth (FIG. 4). With induction of immunity (20 or 40 μM IPTG) a marginal reduction in growth rate (less than 1400 increase in doubling time: FIG. 12) was observed, and about 10000 UBP retention (no detectable loss) in both genes.

Table 1 illustrates sequences described herein.

| | | SEQ ID |
|---|---|---|
| | Sequence | NO: |
| PtNTT2 | MRPYPTIALI SVFLSAATRI SATSSHQASA LPVKKGTHVP | 1 |
| (full length) | DSPKLSKLYI MAKTKSVSSSFDPPRGGSTV APTTPLATGG | |
| | ALRKVRQAVF PIYGNQEVTK FLLIGSIKFF IILALTLTRD | |
| | TKDTLIVTQC GAEAIAFLKI YGVLPAATAF IALYSKMSNA | |
| | MGKKMLFYST CIPFFTFFGLFDVFIYPNAE RLHPSLEAVQ | |
| | AILPGGAASG GMAVLAKIAT HWTSALFYVM AEIYSSVSVG | |
| | LLFWQFANDV VNVDQAKRFY PLFAQMSGLAPVLAGQYVVR | |
| | FASKAVNFEA SMHRLTAAVTFAGIMICIFY QLSSSYVERT | |
| | ESAKPAADNE QSIKPKKKKP KMSMVESGKF LASSQYLRLI | |
| | AMLVLGYGLS INFTEIMWKS LVKKQYPDPL DYQRFMGNFS | |
| | SAVGLSTCIV IFFGVHVIRLLGWKVGALAT PGIMAILALP | |
| | FFACILLGLD SPARLEIAVI FGTIQSLLSK TSKYALFDPT | |
| | TQMAYIPLDD ESKVKGKAAI DVLGSRIGKS GGSLIQQGLV | |
| | FVFGNIINAA PVVGVVYYSVLVAWMSAAGR LSGLFQAQTE | |
| | MDKADKMEAK TNKEK | |
| PtNTT2 (66-575)* | ATGGGTGGTAGCACCGTTGCACCGACCACACCGCTGGCAA | 2 |
| DNA (codon | CCGGTGGTGCACTGCGTAAAGTTCGTCAGGCAGTTTTTCC | |
| optimized) | GATTTATGGCAATCAAGAAGTGACCAAATTTCTGCTGATTG | |
| (*66-575 denotes | GCAGCATCAAATTCTTTATTATCCTGGCACTGACCCTGA | |
| that DNA encoding | CCCGTGATACCAAAGATACCCTGATTGTTACCCAGTGTGGT | |
| the first 65 amino | GCAGAAGCAATTGCATTTCTGAAAATCTATGGTGTTCTG | |
| acid residues have | CCTGCAGCAACCGCATTTATTGCACTGTATAGCAAAATGAG | |
| been deleted | CAACGCAATGGGCAAAAAAATGCTGTTTTATAGCACCTG | |
| relative to the full | TATCCCGTTCTTTACCTTTTTTGGTCTGTTCGATGTGTTCATT | |
| length PtNTT2) | TATCCGAATGCCGAACGTCTGCATCCGAGCCTGGAAG | |
| | CAGTTCAGGCAATTCTGCCTGGTGGTGCCGCAAGCGGTGGT | |
| | ATGGCAGTTCTGGCAAAAATTGCAACCCATTGGACCAGC | |
| | GCACTGTTTTATGTTATGGCAGAAATCTATAGCAGCGTTAG | |
| | CGTTGGTCTGCTGTTTTGGCAGTTTGCAAATGATGTTGT | |
| | TAATGTGGATCAGGCCAAACGTTTTTATCCGCTGTTTGCAC | |
| | AGATGAGCGGTCTGGCACCGGTTCTGGCAGGTCAGTATG | |
| | TTGTTCGTTTTGCAAGCAAAGCCGTTAATTTTGAAGCAAGC | |
| | ATGCATCGTCTGACCGCAGCAGTTACCTTTGCAGGTATT | |
| | ATGATCTGCATCTTTTATCAGCTGAGCAGCTCATATGTTGA | |
| | ACGTACCGAAAGCGCAAAACCGGCAGCAGATAATGAACA | |
| | GAGCATTAAACCGAAGAAAAAAAAACCGAAAATGTCGATG | |
| | GTGGAAAGCGGTAAATTTCTGGCAAGCAGCCAGTATCTGC | |
| | GTCTGATTGCAATGCTGGTTCTGGGTTATGGTCTGAGCATT | |
| | AACTTTACCGAAATCATGTGGAAAAGCCTGGTGAAAAAA | |
| | CAGTATCCGGATCCGCTGGATTATCAGCGTTTTATGGGTAA | |
| | TTTTAGCAGCGCAGTTGGTCTGAGTACCTGCATTGTTAT | |
| | CTTTTTTGGCGTGCATGTTATTCGTCTGCTGGGTTGGAAAGT | |
| | TGGTGCCCTGGCAACACCGGGTATTATGGCCATTCTGG | |
| | CACTGCCGTTTTTTGCATGTATTCTGCTGGGCCTGGATAGTC | |
| | CGGCACGTCTGGAAATTGCAGTTATTTTTGGCACCATT | |
| | CAGAGCCTGCTGAGCAAAACCAGCAAATATGCACTGTTTG | |
| | ATCCGACCACCCAGATGGCATATATCCCGCTGGATGATGA | |
| | AAGCAAAGTTAAAGGCAAAGCAGCCATTGATGTTCTGGGT | |
| | AGCCGTATTGGTAAATCAGGTGGTAGCCTGATTCAGCAGG | |
| | GTCTGGTTTTTGTTTTTGGCAATATTATCAATGCCGCACCGG | |
| | TTGTTGGTGTTGTGTATTATAGCGTTCTGGTTGCATGG | |
| | ATGAGTGCAGCAGGTCGTCTGAGTGGTCTGTTTCAGGCACA | |
| | GACCGAAATGGATAAAGCAGATAAAATGGAAGCCAAAAC | |
| | CAACAAAGAAAAATGA | |
| PtNTT2 (66-575) | ATGGGAGGCAGTACTGTTGCACCAACTACACCGTTGGCAA | 3 |
| DNA (non codon | CCGGCGGTGCGCTCCGCAAAGTGCGACAAGCCGTCTTTCCC | |
| optimized) | ATCTACGGAAACCAAGAAGTCACCAAATTTCTGCTCATCGG | |
| | ATCCATTAAATTCTTTATAATCTTGGCACTCACGCTCACGC | |
| | GTGATACCAAGGACACGTTGATTGTCACGCAATGTGGTGCC | |
| | GAAGCGATTGCCTTTCTCAAAATATACGGGGTGCTACCCGC | |
| | AGCGACCGCATTTATCGCGCTCTATTCCAAAATGTCCAACG | |
| | CCATGGGCAAAAAAATGCTATTTTATTCCACTTGCATTCCT | |
| | TTCTTTACCTTTTTCGGGCTGTTTGATGTTTTCATTTACCCG | |
| | AACGCGGAGCGACTGCACCCTAGTTTGGAAGCCGTGCAGG | |
| | CAATTCTCCCGGGCGGTGCCGCATCTGGCGGCATGGCGGTT | |
| | CTGGCCAAGATTGCGACACACTGGACATCGGCCTTATTTTA | |
| | CGTCATGGCGGAAATATATTCTTCCGTATCGGTGGGGCTAT | |
| | TGTTTTGGCAGTTTGCGAACGACGTCGTCAACGTGGATCAG | |
| | GCCAAGCGCTTTTATCCATTATTTGCTCAAATGAGTGGCCT | |
| | CGCTCCAGTTTTAGCGGGCCAGTATGTGGTACGGTTTGCCA | |
| | GCAAAGCGGTCAACTTTGAGGCATCCATGCATCGACTCACG | |
| | GCGGCCGTAACATTTGCTGGTATTATGATTTGCATCTTTTAC | |
| | CAACTCAGTTCGTCATATGTGGAGCGAACGGAATCAGCAA | |
| | AGCCAGCGGCAGATAACGAGCAGTCTATCAAACCGAAAAA | |
| | GAAGAAACCCAAAATGTCCATGGTTGAATCGGGGAAATTT | |
| | CTCGCGTCAAGTCAGTACCTGCGTCTAATTGCCATGCTGGT | |
| | GCTGGGATACGGCCTCAGTATTAACTTTACCGAAATCATGT | |
| | GGAAAAGCTTGGTGAAGAAACAATATCCAGACCCGCTAGA | |
| | TTATCAACGATTTATGGGTAACTTCTCGTCAGCGGTTGGTTT | |
| | GAGCACATGCATTGTTATTTTCTTCGGTGTGCACGTGATCC | |
| | GTTTGTTGGGGTGGAAAGTCGGAGCGTTGGCTACACCTGGG | |
| | ATCATGGCCATTCTAGCGTTACCCTTTTTTGCTTGCATTTTG | |
| | TTGGGTTTGGATAGTCCAGCACGATTGGAGATCGCCGTAAT | |
| | CTTTGGAACAATTCAGAGTTTGCTGAGCAAAACCTCCAAGT | |
| | ATGCCCTTTTCGACCCTACCACACAAATGGCTTATATTCCTC | |
| | TGGACGACGAATCAAAGGTCAAAGGAAAAGCGGCAATTGA | |
| | TGTTTTGGGATCGCGGATTGGCAAGAGTGGAGGCTCACTGA | |
| | TCCAGCAGGGCTTGGTCTTTGTTTTTGGAAATATCATTAAT | |
| | GCCGCACCTGTAGTAGGGGTTGTCTACTACAGTGTCCTTGT | |
| | TGCGTGGATGAGCGCAGCTGGCCGACTAAGTGGGCTTTTTC | |
| | AAGCACAAACAGAAATGGATAAGGCCGACAAAATGGAGG | |
| | CAAAGACCAACAAAGAAAAGTAG | |
| PtNTT2 (66-575) | MGGSTVAPTTPLATGGALRKVRQAVFPIYGNQEVTKFLLIGSI | 4 |
| protein | KFFIILALTLTRDTKDTLIVTQCGAEMAFLKIYGVLPAATAFIA | |
| | LYSKMSNAMGKKMLFYSTCIPFFTFFGLFDVFIYPNAERLHPS | |
| | LEAVQAILPGGAASGGMAVLAKIATHWTSALFYVMAEIYSSV | |
| | SVGLLFWQFANDVVNVDQAKRFYPLFAQMSGLAPVLAGQYV | |
| | VRFASKAVNFEASMHRLTAAVTFAGIMICIFYQLSSSYVERTE | |
| | SAKPAADNEQSIKPKKKKPKMSMVESGKFLASSQYLRLIAML | |
| | VLGYGLSINFTEIMWKSLVKKQYPDPLDYQRFMGNFSSAVGL | |
| | STCIVIFFGVHVIRLLGWKVGALATPGIMAILALPFFACILLGL | |
| | DSPARLEIAVIFGTIQSLLSKTSKYALFDPTTQMAYIPLDDESK | |
| | VKGKAAIDVLGSRIGKSGGSLIQQGLVFVFGNIINAAPVVGVV | |
| | YYSVLVAWMSAAGRLSGLFQAQTEMDKADKMEAKTNKEK | |
| PtNTT2 (1-22,66- | ATGAGACCATTTCCGACGATTGCCTTGATTTCGGTTTTTCTT | 5 |
| 575)* | TCGGCGGCGACTCGCATTTCGGCAGGAGGCAGTACTGTTGC | |
| DNA | ACCAACTACACCGTTGGCAACCGGCGGTGCGCTCCGCAAA | |
| (*1-22, 66-575 | GTGCGACAAGCCGTCTTTCCCATCTACGGAAACCAAGAAGT | |
| denotes that DNA | CACCAAATTTCTGCTCATCGGATCCATTAAATTCTTTATAAT | |
| encoding amino | CTTGGCACTCACGCTCACGCGTGATACCAAGGACACGTTGA | |
| acid residues 23-65 | TTGTCACGCAATGTGGTGCCGAAGCGATTGCCTTTCTCAAA | |
| have been deleted | ATATACGGGGTGCTACCCGCAGCGACCGCATTTATCGCGCT | |
| relative to the full- | CTATTCCAAAATGTCCAACGCCATGGGCAAAAAAATGCTAT | |
| length PtNTT2) | TTTATTCCACTTGCATTCCTTTCTTTACCTTTTTCGGGCTGTT | |
| | TGATGTTTTCATTTACCCGAACGCGGAGCGACTGCACCCTA | |
| | GTTTGGAAGCCGTGCAGGCAATTCTCCCGGGCGGTGCCGCA | |
| | TCTGGCGGCATGGCGGTTCTGGCCAAGATTGCGACACACTG | |
| | GACATCGGCCTTATTTTACGTCATGGCGGAAATATATTCTT | |
| | CCGTATCGGTGGGGCTATTGTTTTGGCAGTTTGCGAACGAC | |
| | GTCGTCAACGTGGATCAGGCCAAGCGCTTTTATCCATTATT | |
| | TGCTCAAATGAGTGGCCTCGCTCCAGTTTTAGCGGGCCAGT | |
| | ATGTGGTACGGTTTGCCAGCAAAGCGGTCAACTTTGAGGCA | |
| | TCCATGCATCGACTCACGGCGGCCGTAACATTTGCTGGTAT | |
| | TATGATTTGCATCTTTTACCAACTCAGTTCGTCATATGTGGA | |
| | GCGAACGGAATCAGCAAAGCCAGCGGCAGATAACGAGCA | |
| | GTCTATCAAACCGAAAAAGAAGAAACCCAAAATGTCCATG | |
| | GTTGAATCGGGGAAATTTCTCGCGTCAAGTCAGTACCTGCG | |
| | TCTAATTGCCATGCTGGTGCTGGGATACGGCCTCAGTATTA | |
| | ACTTTACCGAAATCATGTGGAAAAGCTTGGTGAAGAAACA | |
| | ATATCCAGACCCGCTAGATTATCAACGATTTATGGGTAACT | |
| | TCTCGTCAGCGGTTGGTTTGAGCACATGCATTGTTATTTTCT | |
| | TCGGTGTGCACGTGATCCGTTTGTTGGGGTGGAAAGTCGGA | |
| | GCGTTGGCTACACCTGGGATCATGGCCATTCTAGCGTTACC | |
| | CTTTTTTGCTTGCATTTTGTTGGGTTTGGATAGTCCAGCACG | |
| | ATTGGAGATCGCCGTAATCTTTGGAACAATTCAGAGTTTGC | |
| | TGAGCAAAACCTCCAAGTATGCCCTTTTCGACCCTACCACA | |
| | CAAATGGCTTATATTCCTCTGGACGACGAATCAAAGGTCAA | |
| | AGGAAAAGCGGCAATTGATGTTTTGGGATCGCGGATTGGC | |
| | AAGAGTGGAGGCTCACTGATCCAGCAGGGCTTGGTCTTTGT | |
| | TTTTGGAAATATCATTAATGCCGCACCTGTAGTAGGGGTTG | |
| | TCTACTACAGTGTCCTTGTTGCGTGGATGAGCGCAGCTGGC | |
| | CGACTAAGTGGGCTTTTTCAAGCACAAACAGAAATGGATA | |
| | AGGCCGACAAAATGGAGGCAAAGACCAACAAAGAAAAGT | |
| | AG | |
| PtNTT2(1-22, 66- | MRPFPTIALISVFLSAATRISAGGSTVAPTTPLATGGALRKVRQ | 6 |
| 575) | AVFPIYGNQEVTKFLLIGSIKFFIILALTLTRDTKDTLIVTQCGA | |
| protein | EATAFLKIYGVLPAATAFIALYSKMSNAMGKKMLFYSTCIPFF | |
| | TFFGLFDVFIYPNAERLHPSLEAVQAILPGGAASGGMAVLAKI | |
| | ATHWTSALFYVMAEIYSSVSVGLLFWQFANDVVNVDQAKRF | |
| | YPLFAQMSGLAPVLAGQYVVRFASKAVNFEASMHRLTAAVT | |
| | FAGIMICIFYQLSSSYVERTESAKPAADNEQSIKPKKKKPKMS | |
| | MVESGKFLASSQYLRLIAMLVLGYGLSINFTEIMWKSLVKKQ | |
| | YPDPLDYQRFMGNFSSAVGLSTCIVIFFGVHVIRLLGWKVGAL | |
| | ATPGIMAILALPFFACILLGLDSPARLEIAVIFGTIQSLLSKTSKY | |
| | ALFDPTTQMAYIPLDDESKVKGKAAIDVLGSRIGKSGGSLIQQ | |
| | GLVFVFGNIINAAPVVGVVYYSVLVAWMSAAGRLSGLFQAQ | |
| | TEMDKADKMEAKTNKEK | |
| PtNTT2 (23-575)* | ATGACTTCCTCTCATCAAGCAAGTGCACTTCCTCTCAAAAA | 7 |
| DNA | GGGAACGCATGTCCCGGACTCTCCGAAGTTGTCAAAGCTAT | |
| (*23-575 denotes | ATATCATGGCCAAAACCAAGAGTGTATCCTCGTCCTTCGAC | |
| that DNA encoding | CCCCCTCGGGGAGGCAGTACTGTTGCACCAACTACACCGTT | |
| amino acid residues | GGCAACCGGCGGTGCGCTCCGCAAAGTGCGACAAGCCGTC | |
| 1-22 have been | TTTCCCATCTACGGAAACCAAGAAGTCACCAAATTTCTGCT | |
| deleted relative to | CATCGGATCCATTAAATTCTTTATAATCTTGGCACTCACGCT | |
| the full-length | CACGCGTGATACCAAGGACACGTTGATTGTCACGCAATGTG | |
| PtNTT2) | GTGCCGAAGCGATTGCCTTTCTCAAAATATACGGGGTGCTA | |
| | CCCGCAGCGACCGCATTTATCGCGCTCTATTCCAAAATGTC | |
| | CAACGCCATGGGCAAAAAAATGCTATTTTATTCCACTTGCA | |
| | TTCCTTTCTTTACCTTTTTCGGGCTGTTTGATGTTTTCATTTA | |
| | CCCGAACGCGGAGCGACTGCACCCTAGTTTGGAAGCCGTG | |
| | CAGGCAATTCTCCCGGGCGGTGCCGCATCTGGCGGCATGGC | |
| | GGTTCTGGCCAAGATTGCGACACACTGGACATCGGCCTTAT | |
| | TTTACGTCATGGCGGAAATATATTCTTCCGTATCGGTGGGG | |
| | CTATTGTTTTGGCAGTTTGCGAACGACGTCGTCAACGTGGA | |
| | TCAGGCCAAGCGCTTTTATCCATTATTTGCTCAAATGAGTG | |
| | GCCTCGCTCCAGTTTTAGCGGGCCAGTATGTGGTACGGTTT | |
| | GCCAGCAAAGCGGTCAACTTTGAGGCATCCATGCATCGACT | |
| | CACGGCGGCCGTAACATTTGCTGGTATTATGATTTGCATCT | |
| | TTTACCAACTCAGTTCGTCATATGTGGAGCGAACGGAATCA | |
| | GCAAAGCCAGCGGCAGATAACGAGCAGTCTATCAAACCGA | |
| | AAAAGAAGAAACCCAAAATGTCCATGGTTGAATCGGGGAA | |
| | ATTTCTCGCGTCAAGTCAGTACCTGCGTCTAATTGCCATGC | |
| | TGGTGCTGGGATACGGCCTCAGTATTAACTTTACCGAAATC | |
| | ATGTGGAAAAGCTTGGTGAAGAAACAATATCCAGACCCGC | |
| | TAGATTATCAACGATTTATGGGTAACTTCTCGTCAGCGGTT | |
| | GGTTTGAGCACATGCATTGTTATTTTCTTCGGTGTGCACGTG | |
| | ATCCGTTTGTTGGGGTGGAAAGTCGGAGCGTTGGCTACACC | |
| | TGGGATCATGGCCATTCTAGCGTTACCCTTTTTTGCTTGCAT | |
| | TTTGTTGGGTTTGGATAGTCCAGCACGATTGGAGATCGCCG | |
| | TAATCTTTGGAACAATTCAGAGTTTGCTGAGCAAAACCTCC | |
| | AAGTATGCCCTTTTCGACCCTACCACACAAATGGCTTATAT | |
| | TCCTCTGGACGACGAATCAAAGGTCAAAGGAAAAGCGGCA | |
| | ATTGATGTTTTGGGATCGCGGATTGGCAAGAGTGGAGGCTC | |
| | ACTGATCCAGCAGGGCTTGGTCTTTGTTTTTGGAAATATCA | |
| | TTAATGCCGCACCTGTAGTAGGGGTTGTCTACTACAGTGTC | |
| | CTTGTTGCGTGGATGAGCGCAGCTGGCCGACTAAGTGGGCT | |
| | TTTTCAAGCACAAACAGAAATGGATAAGGCCGACAAAATG | |
| | GAGGCAAAGACCAACAAAGAAAAGTAG | |
| PtNTT2 (23-575) | MTSSHQASALPLKKGTHVPDSPKLSKLYIMAKTKSVSSSFDPP | 8 |
| protein | RGGSTVAPTTPLATGGALRKVRQAVFPIYGNQEVTKFLLIGSI | |
| | KFFIILALTLTRDTKDTLIVTQCGAEMAFLKIYGVLPAATAFIA | |
| | LYSKMSNAMGKKMLFYSTCIPFFTFFGLFDVFIYPNAERLHPS | |
| | LEAVQAILPGGAASGGMAVLAKIATHWTSALFYVMAEIYSSV | |
| | SVGLLFWQFANDVVNVDQAKRFYPLFAQMSGLAPVLAGQYV | |
| | VRFASKAVNFEASMHRLTAAVTFAGIMICIFYQLSSSYVERTE | |
| | SAKPAADNEQSIKPKKKKPKMSMVESGKFLASSQYLRLIAML | |
| | VLGYGLSINFTEIMWKSLVKKQYPDPLDYQRFMGNFSSAVGL | |
| | STCIVIFFGVHVIRLLGWKVGALATPGIMAILALPFFACILLGL | |
| | DSPARLEIAVIFGTIQSLLSKTSKYALFDPTTQMAYIPLDDESK | |
| | VKGKAAIDVLGSRIGKSGGSLIQQGLVFVFGNIINAAPVVGVV | |
| | YYSVLVAWMSAAGRLSGLFQAQTEMDKADKMEAKTNKEK | |
| pBRX2* | AACGCCAGCAACGCGGCCTTTTTACGGTTCCTGGCCTTTTG | 9 |
| (*N denotes the | CTGGCCTTTTGCTCACATGTTCTTTCCTGCGTTATCCCC | |
| position of the | TGATTCTGTGGATAACCGTATTACCGCCTTTGAGTGAGCTG | |
| UBP) | ATACCGCTCGCCGCAGCCGAACGACCGAGCGCAGCGAGT | |
| | CAGTGAGCGAGGAAGCGGAAGAGCGCCTGATGCGGTATTT | |
| | TCTCCTTACGCATCTGTGCGGTATTTCACACCGCATATGG | |
| | TGCACTCTCAGTACAATCTGCTCTGATGCCGCATAGTTAAG | |
| | CCAGTATACACTCCGCTATCGCTACGTGACTGGGTCATG | |
| | GCTGCGCCCCGACACCCGCCAACACCCGCTGACGCGCCCTG | |
| | ACGGGCTTGTCTGCTCCCGGCATCCGCTTACAGACAAGC | |
| | TGTGACCGTCTCCGGGAGCTGCATGTGTCAGAGGTTTTCAC | |
| | CGTCATCACCGAAACGCGCGAGGCAGCTGCGGTAAAGCT | |
| | CATCAGCGTGGTCGTGAAGCGATTCACAGATGTCTGCCTGT | |
| | TCATCCGCGTCCAGCTCGTTGAGTTTCTCCAGAAGCGTT | |
| | AATGTCTGGCTTCTGATAAAGCGGGCCATGTTAAGGGCGGT | |
| | TTTTTCCTGTTTGGTCACTGATGCCTCCGTGTAAGGGGG | |
| | ATTTCTGTTCATGGGGGTAATGATACCGATGAAACGAGAG | |
| | AGGATGCTCACGATACGGGTTACTGATGATGAACATGCCC | |
| | GGTTACTGGAACGTTGTGAGGGTAAACAACTGGCGGTATG | |
| | GATGCGGCGGGACCAGAGAAAAATCACTCAGGGTCAATGC | |
| | CAGCGCTTCGTTAATACAGATGTAGGTGTTCCACAGGGTAG | |
| | CCAGCAGCATCCTGCGATGCAGATCCGGAACATAATGGT | |
| | GCAGGGCGCTGACTTCCGCGTTTCCAGACTTTACGAAACAC | |
| | GGAAACCGAAGACCATTCATGTTGTTGCTCAGGTCGCAG | |
| | ACGTTTTGCAGCAGCAGTCGCTTCACGTTCGCTCGCGTATC | |
| | GGTGATTCATTCTGCTAACCAGTAAGGCAACCCCGCCAG | |
| | CCTAGCCGGGTCCTCAACGACAGGAGCACGATCATGCGCA | |
| | CCCGTGGCCAGGACCCAACGCTGCCCGAAATTCTTGAAGA | |
| | CGAAAGGGCCTCGTGATACGCCTATTTTTATAGGTTAATGT | |
| | CATGATAATAATGGTTTCTTAGACGTCAGGTGGCACTTT | |
| | TCGGGGAAATGTGCGCGGAACCCCTATTTGTTTATTTTTCT | |
| | AAATACATTCAAATATGTATCCGCTCATGAGACAATAAC | |
| | CCTGATAAATGCTTCAATAATATTGAAAAAGGAAGAGTAT | |
| | GAGTATTCAACATTTCCGTGTCGCCCTTATTCCCTTTTTT | |
| | GCGGCATTTTGCCTTCCTGTTTTTGCTCACCCAGAAACGCTG | |
| | GTGAAAGTAAAAGATGCTGAAGATCAGTTGGGTGCACG | |
| | AGTGGGTTACATCGAACTGGATCTCAACAGCGGTAAGATC | |
| | CTTGAGAGTTTTCGCCCCGAAGAACGTTTTCCAATGATGA | |
| | GCACTTTTAAAGTTCTGCTATGTGGCGCGGTATTATCCCGT | |
| | GTTGACGCCGGGCAAGAGCAACTCGGTCGCCGCATACAC | |
| | TATTCTCAGAATGACTTGGTTGAGTACTCACCAGTCACAGA | |
| | AAAGCATCTTACGGATGGCATGACAGTAAGAGAATTATG | |
| | CAGTGCTGCCATAACCATGAGTGATAACACTGCGGCCAACT | |
| | TACTTCTGACAACGATCGGAGGACCGAAGGAGCTAACCG | |
| | CTTTTTTGCACAACATGGGGGATCATGTAACTCGCCTTGAT | |
| | CGTTGGGAACCGGAGCTGAATGAAGCCATACCAAACGAC | |
| | GAGCGTGACACCACGATGCCTGCAGCAATGGCAACAACGT | |
| | TGCGCAAACTATTAACTGGCGAACTACTTACTCTAGCTTC | |
| | CCGGCAACAATTAATAGACTGGATGGAGGCGGATAAAGTT | |
| | GCAGGACCACTTCTGCGCTCGGCCCTTCCGGCTGGCTGGT | |
| | TTATTGCTGATAAATCTGGAGCCGGTGAGCGTGGCTCTCGC | |
| | GGTATCATTGCAGCACTGGGGCCAGATGGTAAGCCCTCC | |
| | CGTATCGTAGTTATCTACACGACGGGGAGTCAGGCAACTAT | |
| | GGATGAACGAAATAGACAGATCGCTGAGATAGGTGCCTC | |
| | ACTGATTAAGCATTGGTAACTGTCAGACCAAGTTTACTCAT | |
| | ATATACTTTAGATTGATTTAAAACTTCATTTTTAATTTA | |
| | AAAGGATCTAGGTGAAGATCCTTTTTGATAATCTCATGACC | |
| | AAAATCCCTTAACGTGAGTTTTCGTTGGCTTTACACTTT | |
| | ATGCTTCCGGCTCGTATGTTGTGTGGAANTGTGAGCGGATA | |
| | ACAATTTCACACAGGAAACAGCCACTGAGCGTCAGACCC | |
| | CGTAGAAAAGATCAAAGGATCTTCTTGAGATCCTTTTTTTC | |
| | TGCGCGTAATCTGCTGCTTGCAAACAAAAAAACCACCGC | |
| | TACCAGCGGTGGTTTGTTTGCCGGATCAAGAGCTACCAACT | |
| | CTTTTTCCGAAGGTAACTGGCTTCAGCAGAGCGCAGATA | |
| | CCAAATACTGTCCTTCTAGTGTAGCCGTAGTTAGGCCACCA | |
| | CTTCAAGAACTCTGTAGCACCGCCTACATACCTCGCTCT | |
| | GCTAATCCTGTTACCAGTGGCTGCTGCCAGTGGCGATAAGT | |
| | CGTGTCTTACCGGGTTGGACTCAAGACGATAGTTACCGG | |
| | ATAAGGCGCAGCGGTCGGGCTGAACGGGGGGTTCGTGCAC | |
| | ACAGCCCAGCTTGGAGCGAACGACCTACACCGAACTGAGA | |
| | TACCTACAGCGTGAGCTATGAGAAAGCGCCACGCTTCCCG | |
| | AAGGGAGAAAGGCGGACAGGTATCCGGTAAGCGGCAGGG | |
| | TCGGAACAGGAGAGCGCACGAGGGAGCTTCCAGGGGGAA | |
| | ACGCCTGGTATCTTTATAGTCCTGTCGGGTTTCGCCACCTCT | |
| | GACTTGAGCGTCGATTTTTGTGATGCTCGTCAGGGGGGCGG | |
| | AGCCTATGGAAA | |

TABLE 2

| | | | |
|---|---|---|---|
| | | | SEQ ID |
| Primer | Application | Sequence | NO: |

Transporter plasmid cloning and chromosomal integration

| | | | |
|---|---|---|---|
| YZ552 | Cloning | GGGAGGCAGTACTGTTGCAC | 10 |
| | PtNTT2(66-575) | | |
| pCDF- | Cloning | GGTATATCTCCTTATTAAAGTTAAACAAAATTATTTCT | 11 |
| 1b-fwd | PtNTT2(66-575) | ACAGGGG | |
| T7 seq | sequencing | TAATACGACTCACTATAGGG | 12 |
| | pCDF plasmids | | |
| T7 term | sequencing | GCTAGTTATTGCTCAGCGG | 13 |
| seq | pCDF plasmids | | |
| YZ580 | transporter | TTACATTAATTGCGTTGCGCTC | 14 |
| | cloning | | |
| DM002 | transporter | TTTTGGCGGATGGCATTTGAGAAGCACACGG | 15 |
| | cloning | | |
| YZ576 | transporter | ATTCTCACCAATAAAAAACGCCCGG | 16 |
| | cloning | | |
| YZ581 | transporter | CCTGTAGAAATAATTTTGTTTAACTTTAATAAGGAG | 17 |
| | cloning | | |
| DM052 | transporter | CCCCGCGCGTTGGCCGATTC | 18 |
| | sequencing | | |
| DM053 | transporter | GAAGGGCAATCAGCTGTTG | 19 |
| | sequencing | | |
| YZ50 | transporter | CAGGGCAGGGTCGTTAAATAG | 20 |
| | sequencing | | |
| YZ584 | lacI promoter | GACACCATCGAATGGCGCAAAACCTTTCGCGGTATGG | 21 |
| | | CATGATAGCGCCCGG | |
| YZ585 | lacI promoter | CCGGGCGCTATCATGCCATACCGCGAAAGGTTTTGCG | 22 |
| | | CCATTCGATGGTGTC | |
| YZ582 | bla promoter | ATTTTTCTAAATACATTCAAATATGTATCCGCTCATGA | 23 |
| | | GACAATAACCCTG | |
| YZ583 | bla promoter | CAGGGTTATTGTCTCATGAGCGGATACATATTTGAAT | 24 |
| | | GTATTTAGAAAAAT | |
| YZ599 | lac promoter | TTAGGCACCCCAGGCTTTACACTTTATGCTTCCGGCTG | 25 |
| | | GTATGTTGTGTGGA | |
| YZ600 | lac promoter | TCCACACAACATACCAGCCGGAAGCATAAAGTGTAAA | 26 |
| | | GCCTGGGGTGCCTAA | |
| YZ595 | lacUV5 | CTAGGCACCCCAGGCTTTACACTTTATGCTTCCGGCTG | 27 |
| | promoter | GTATAATGTGTGGA | |
| YZ596 | lacUV5 | TCCACACATTATACCAGCCGGAAGCATAAAGTGTAAA | 28 |
| | promoter | GCCTGGGGTGCCTAG | |
| YZ7 | transporter | GGGGATAACGCAGGAAAGAACATG | 29 |
| | integration | | |
| | cloning | | |
| YZ12 | transporter | GCACTTTTCGGGGAAATGTGCG | 30 |
| | integration | | |
| | cloning | | |
| YZ612 | transporter | AATTGCGGCCTATATGGATGTTGGAACCGTAAGAGAA | |
| | integration | ATAGACAGGCGGTCCTGTGACGGAAGATCACTTCGCA | 31 |
| | cloning | G | |
| YZ613 | transporter | TGCTCACATGTTCTTTCCTGCGTTATCCCCGCGTGGTG | 32 |
| | integration | AACCAGGC | |
| | cloning | | |
| YZ614 | transporter | ACCGCCTGTCTATTTCTCTTACGGTTCC | 33 |
| | integration | | |
| | cloning | | |
| YZ615 | transporter | CGCGCTTAATGCGCCGCTACAGGGCGCGTCGATTGGT | 34 |
| | integration | GCCAGCGCGCAG | |
| | cloning | | |
| YZ610 | transporter | GGTATATCTCCTTATTAAAGTTAAACAAAATTATTTCT | 35 |
| | integration | ACAGG | |
| | cloning | | |
| YZ616 | lacZYA:: | CAGCCACGTTTCTGCGAAAAC | 36 |
| | transporter | | |
| | integration | | |
| YZ617 | lacZYA:: | TACAGCGGTTCCTTACTGGC | 37 |
| | transporter | | |
| | integration | | |
| YZ618 | transporter | GGGTGGTGAATGTGAAACCAGTAACG | 38 |
| | integration | | |
| | colony PCR | | |
| YZ619 | transporter | CTGGGTGTTTACTTCGGTCTG | 39 |
| | integration | | |
| | colony PCR | | |
| YZ69 | transporter | GGCCGTAATATCCAGCTGAAC | 40 |
| | integration | | |
| | colony PCR | | |
| YZ587 | transporter | ACTAGGGTGCAGTCGCTCCG | 41 |
| | integration | | |
| | colony PCR | | |
| pCDF- | integration | TTAACCTAGGCTGCTGCCACCG | 42 |
| 1b-rev | cloning | | |
| YZ703 | tac promoter | GCGCAACGCAATTAATGTAATTCTGAAATGAGCTGTT | 43 |
| | | GACAATTAATCATCGGCTCG | |
| YZ704 | tac promoter | AACAAAATTATTTCTACAGGTCCACACATTATACGAG | 44 |
| | | CCGATGATTAATTGTCAAC | |
| YZ707 | N25 promoter | GCGCAACGCAATTAATGTAATCATAAAAAATTTATTT | 45 |
| | | GCTTTCAGGAAAATTTTTCTG | |
| YZ708 | N25 promoter | AACAAAATTATTTCTACAGGTGAATCTATTATACAGA | 46 |
| | | AAAATTTTCCTGAAAGCAAATA | |
| YZ709 | λ promoter | GCGCAACGCAATTAATGTAATTATCTCTGGCGGTGTT | 47 |
| | | GACATAAATACCACTGGCG | |
| YZ710 | λ promoter | AACAAAATTATTTCTACAGGTGTGCTCAGTATCACCG | 48 |
| | | CCAGTGGTATTTATGTCAAC | |
| YZ711 | H207 promtoer | GCGCAACGCAATTAATGTAATTTTAAAAAATTCATTT | 49 |
| | | GCTAAACGCTTCAAATTCTCG | |
| YZ712 | H207 promtoer | AACAAAATTATTTCTACAGGTGAAGTATATTATACGA | |
| | | GAATTTGAAGCGTTTAGC | 50 |

pUCX and pBRX Golden Gate destination plasmid cloning

| | | | |
|---|---|---|---|
| pUC19- | TK1 site | TGGGGTGCCTAATGAGTGAGC | 51 |
| lin-fwd | removal/pBRX1 | | |
| | linearization | | |
| pUC19- | TK1 site | CTATGACCATGATTACGCCAAGCTTG | 52 |
| lin-rev | removal/pBRX1 | | |
| | linearization | | |
| YZ51 | bla BsaI site | TCTCGCGGTATCATTGCAGCACTG | 53 |
| | mutation | | |
| YZ52 | bla BsaI site | GCCACGCTCACCGGCTCC | 54 |
| | mutation | | |
| YZ95 | pUCX2/pBRX2 | AACGAAAACTCACGTTAAGGG | 55 |
| | linearization | | |
| YZ96 | pUCX2/pBRX2 | CCACTGAGCGTCAGACC | 56 |
| | linearization | | |
| YZ93 | BsaI zeoR stuffer | GAGACCCGTCGTTGACAATTAATCATCGGC | 57 |
| | cassette | | |
| YZ94 | BsaI zeoR stuffer | GAGACCATTCTCACCAATAAAAAACGCCCGG | 58 |
| | cassette | | |

pCas9 and pAIO cloning, Cas9 chromosomal integration

| | | | |
|---|---|---|---|
| JL126 | pCas9 cloning | CGGGGTACCATGGACAAGAAGTACTCCATT | 59 |
| JL128 | pCas9 cloning | CTAGTCTAGATTACACCTTCCTCTTCTTCTTGGG | 60 |
| BL557 | pCas9 BsmBI | CTCCGGGGAAACCGCCGAAGCCACGCGGCTCAA | 61 |
| | site removal | | |
| BL558 | pCas9 BsmBI | CTTCGGCGGTTTCCCCGGAGTCGAACAGGAGGGCGCC | |
| | site removal | AATGAGG | 62 |
| BL559 | pCas9-Multi | AGGAAGAAGACGTCTCACGCATCTTACTGCGCAGATA | 63 |
| | cloning | CGC | |
| BL560 | pCas9-Multi | AAGATGCGTGAGACGTCTTCTTCCTCGTCTCGGTCGAC | 64 |
| | cloning | AGTTCATAGGTGATTGCTCAGG | |
| YZ720 | arsB::Cas9 | GTCCCAAATCGCAGCCAATCACATTG | 65 |
| | integration | | |
| YZ721 | arsB::Cas9 | GTCCTGACCATCGTATTGGTTATCTGGC | 66 |
| | integration | | |
| TG1 | Cas9 sequencing | ATTTAGAGGGCAGTGCCAGCTCGTTA | 67 |
| TG2 | Cas9 sequencing | CTGCATTCAGGTAGGCATCATGCGCA | 68 |
| TG3 | Cas9 sequencing | CTGGGCTACCTGCAAGATTAGCGATG | 69 |
| TG4 | Cas9 sequencing | TGAAGGACTGGGCAGAGGCCCCCTT | 70 |
| TG5 | Cas9 sequencing | CGTAGGTGTCTTTGCTCAGTTGAAGC | 71 |
| TG6 | Cas9 sequencing | TAGCCATCTCATTACTAAAGATCTCCT | 72 |
| BL731 | pAIO-Multi | CGATATCGTTGGTCTCAACGACACAATTGTAAAGGTT | 73 |
| | cloning (ΔCas9, | AGATCT | |
| | introduce BsaI) | | |
| BL732 | pAIO-Multi | CAACGATATCGGTCTCACACTGACTGGGCCTTTCGTTT | 74 |
| | cloning (ΔCas9, | TATCT | |
| | introduce BsaI) | | |
| BL450 | pAIO guide | GCAATCACCTATGAACTGTCGAC | 75 |
| | sequencing | | |

Cas9 guide cloning
upper case denotes the BsmBI recognition sequence (6 nt), the BsmBI restriction site
overhang (4 nt), or variable target (spacer) sequence (18-20 nt)

| | | | |
|---|---|---|---|
| BL691 | 1st sgRNA GG | aggaggaaggaCGTCTCaTGCGccccgcattCACACAATGTAGTG | 76 |
| | A GXG-T | ATCAgttttagagctagaaatagc | |
| BL627 | 1st sgRNA GG | aggaggaaggaCGTCTCaTGCGccccgcattCCAGGATGGGTAC | 77 |
| | A GXA-T | CACCCgttttagagctagaaatagc | |
| BL707 | 1st sgRNA GG | aggaggaaggaCGTCTCaTGCGccccgcattCACACAATGTAGTC | 78 |
| | A GXC-T | ATCAgttttagagctagaaatagc | |
| BL642 | 1st sgRNA GG | aggaggaaggaCGTCTCaTGCGccccgcattCCAGGATGGGCAC | 79 |
| | A GXT-G | CAGCCgttttagagctagaaatagc | |
| BL659 | 1st sgRNA GG | aggaggaaggaCGTCTCaTGCGccccgcattCCATGATGGGCACC | 80 |
| | A AXG-T/CXT- | ACCCgttttagagctagaaatagc | |
| | T | | |
| BL623 | 1st sgRNA GG | aggaggaaggaCGTCTCaTGCGccccgcattCACACAATGTATAG | 81 |
| | A AXA-T | ATCAgttttagagctagaaatagc | |
| BL628 | 1st sgRNA GG | aggaggaaggaCGTCTCaTGCGccccgcattCCAGGATGGGCAC | 82 |
| | A AXC-T | CATCCgttttagagctagaaatagc | |
| BL567 | 1st sgRNA GG | aggaggaaggaCGTCTCaTGCGccccgcattGTTGTGTGGAAATG | 83 |
| | A AXT-A | TGAGgttttagagctagaaatagc | |
| BL593 | 1st sgRNA GG | aggaggaaggaCGTCTCaTGCGccccgcattTGTCACTACTCTGA | 84 |
| | A CXG-G | CCAGgttttagagctagaaatagc | |
| BL639 | 1st sgRNA GG | aggaggaaggaCGTCTCaTGCGccccgcattTGTCACTACTCTGA | 85 |
| | A CXA-A | CCAAgttttagagctagaaatagc | |
| BL693 | 1st sgRNA GG | aggaggaaggaCGTCTCaTGCGccccgcattCACACAATGTACTC | 86 |
| | A CXC-T | ATCAgttttagagctagaaatagc | |
| BL660 | 1st sgRNA GG | aggaggaaggaCGTCTCaTGCGccccgcattCCAAGATGGGCAC | |
| | A CXT-A | CACCCgttttagagctagaaatagc | 87 |
| BL695 | 1st sgRNA GG | aggaggaaggaCGTCTCaTGCGccccgcattCACACAATGTATTG | 88 |
| | A TXG-T | ATCAgttttagagctagaaatagc | |
| BL629 | 1st sgRNA GG | aggaggaaggaCGTCTCaTGCGccccgcattCACACAATGTATAA | 89 |
| | A TXA-A | ATCAgttttagagctagaaatagc | |
| BL657 | 1st sgRNA GG | aggaggaaggaCGTCTCaTGCGccccgcattCCAGGATGGGGAC | 90 |
| | A TXC-G | CACCCgttttagagctagaaatagc | |
| BL620 | 1st sgRNA GG | aggaggaaggaCGTCTCaTGCGccccgcattTTCACAATACTTTC | 91 |
| | A TXT-T | TTTAgthtagagctagaaatagc | |
| BL701 | 2nd sgRNA, | gcattTCACACAATGTAGGATCAgttttagagctagaaatagc | 92 |
| | Fwd GΔG | | |
| BL702 | 2nd sgRNA, Rev | TGATCCTACATTGTGTGAaatgcggggcgcatcttact | 93 |
| | GΔG | | |
| BL617 | 2nd sgRNA, | gcattACCAGGATGGGACCACCCgttttagagctagaaatagc | 94 |
| | Fwd GΔA | | |
| BL618 | 2nd sgRNA, Rev | GGGTGGTCCCATCCTGGTaatgcggggcgcatcttact | 95 |
| | GΔA | | |
| BL705 | 2nd sgRNA, | gcattTCACACAATGTAGCATCAgttttagagctagaaatagc | 96 |
| | Fwd GΔC | | |
| BL706 | 2nd sgRNA, Rev | TGATGCTACATTGTGTGAaatgcggggcgcatcttact | 97 |
| | GΔC | | |
| BL614 | 2nd sgRNA, | gcattACCAGGATGGGCACCACCgttttagagctagaaatagc | 98 |
| | Fwd GΔT | | |
| BL615 | 2nd sgRNA, Rev | GGTGGTGCCCATCCTGGTaatgcggggcgcatcttact | 99 |
| | GΔT | | |
| BL682 | 2nd sgRNA, | gcattACCAGATGGGCACCACCCgttttagagctagaaatagc | 100 |
| | Fwd AΔG | | |
| BL683 | 2nd sgRNA, Rev | GGGTGGTGCCCATCTGGTaatgcggggcgcatcttact | 101 |
| | AΔG | | |
| BL575 | 2nd sgRNA, | gcattTCACACAATGTAAGATCAgttttagagctagaaatagc | 102 |
| | Fwd AΔA | | |
| BL576 | 2nd sgRNA, Rev | TGATCTTACATTGTGTGAaatgcggggcgcatcttact | 103 |
| | AΔA | | |
| BL564 | 2nd sgRNA, | gcattTGTTGTGTGGAATGTGAGgttttagagctagaaatagc | 104 |
| | Fwd AΔT | | |
| BL565 | 2nd sgRNA, Rev | CTCACATTCCACACAACAaatgcggggcgcatcttact | 105 |
| | AΔT | | |
| BL675 | 2nd sgRNA, | gcattTTGTCACTACTCTGACCGgttttagagctagaaatagc | 106 |
| | Fwd CΔG | | |
| BL676 | 2nd sgRNA, Rev | CGGTCAGAGTAGTGACAAaatgcggggcgcatcttact | 107 |
| | CΔG | | |
| BL673 | 2nd sgRNA, | gcattTTGTCACTACTCTGACCAgttttagagctagaaatagc | 108 |
| | Fwd CΔA | | |
| BL674 | 2nd sgRNA, Rev | TGGTCAGAGTAGTGACAAaatgcggggcgcatcttact | 109 |
| | CΔA | | |
| BL703 | 2nd sgRNA, | gcattTCACACAATGTACCATCAgttttagagctagaaatagc | 110 |
| | Fwd CΔC | | |
| BL704 | 2nd sgRNA, Rev | TGATGGTACATTGTGTGAaatgcggggcgcatcttact | 111 |
| | CΔC | | |
| BL697 | 2nd sgRNA, | gcattTCACACAATGTATGATCAgttttagagctagaaatagc | 112 |
| | Fwd TΔG | | |
| BL698 | 2nd sgRNA, Rev | TGATCATACATTGTGTGAaatgcggggcgcatcttact | 113 |
| | TΔG | | |
| BL679 | 2nd sgRNA, | gcattTCACACAATGTATAATCAgttttagagctagaaatagc | 114 |
| | Fwd TΔA | | |
| BL680 | 2nd sgRNA, Rev | TGATTATACATTGTGTGAaatgcggggcgcatcttact | 115 |
| | TΔA | | |
| BL620 | 2nd sgRNA, | gcattATTCACAATACTTCTTTAgttttagagctagaaatagc | 116 |
| | Fwd TΔT | | |
| BL621 | 2nd sgRNA, Rev | TAAAGAAGTATTGTGAATaatgcggggcgcatcttact | 117 |
| | TΔT | | |
| BL562 | 1st sgRNA | agaaggaagaCGTCTCaCTGTcgaccaaaaaagcctgctcgttgagc | 118 |
| | construction | | |
| BL563 | 2nd sgRNA | aagaaggaCGTCTCaACAGtagtggcagcggctaactaag | 119 |
| | construction | | |
| BL566 | Terminating | aggagaggaCGTCTCtCGACcaaaaaagcctgctcgttgagcag | 120 |
| | sgRNA | | |
| | construction | | |
| BL514 | natural hEGFP | agtaagatgcgccccgcattGACCAGGATGGGCACCACCCgttttaga | 121 |
| | guide cloning | gctagaaatag | |
| BL515 | natural hEGFP | ctatttctagctctaaaacGGGTGGTGCCCATCCTGGTCaatgcggggc | 122 |
| | guide cloning | gcatcttact | |
| BL464 | natural hEGFP | GTTTTAGAGCTAGAAATAGCAAGTTAAAATAAGGCTA | 123 |
| | guide cloning | GT | |
| BL465 | natural hEGFP | AATGCGGGGCGCATCTTACT | 124 |
| | guide cloning | | |
| YZ310 | ESer-69 guide | cattGGCACCGGTCTACTAAAC | 125 |
| | fwd | | |
| YZ316 | ESer-69 guide | aaacGTTTAGTAGACCGGTGCC | 126 |
| | rev | | |
| YZ359 | GFP151-69 | cgcattCACACAATGTAAGTATCAgtttt | 127 |
| | guide fwd | | |
| YZ360 | GFP151-69 | ctctaaaacTGATACTTACATTGTGTGaa | 128 |
| | guide rev | | |

In vitro cleavage (IVC) DNA templates and sgRNAs

| | | | |
|---|---|---|---|
| BL487 | IVC DNA | GTTTACGTCGCCGTCCAGCTCGACCAGGATGGGCACC | 129 |
| | Template | AACCCGGTGAACAGCTCCTCGCC | |
| BL488 | IVC DNA | GTTTACGTCGCCGTCCAGCTCGACCAGGATGGGCACC | 130 |
| | Template | AGCCCGGTGAACAGCTCCTCGCC | |
| BL489 | IVC DNA | GTTTACGTCGCCGTCCAGCTCGACCAGGATGGGCACC | 131 |
| | Template | ATCCCGGTGAACAGCTCCTCGCC | |
| BL408 | IVC DNA | GTTTACGTCGCCGTCCAGCTCGACCAGGATGGGCACC | 132 |
| | Template | ACCCCGGTGAACAGCTCCTCGCC | |
| BL415 | IVC Template | GCATCGCCCTCGCCCTCGCCGGACACGCTGAACTTGT | 133 |
| | Extension | GGCCGTTTACGTCGCCGTCCAGC | |
| BL416 | IVC Template | TGCAGTTTCATTTGATGCTCGATGAGTTATGGTGAGCA | 134 |
| | Extension | AGGGCGAGGAGCTGTTCACCGG | |
| BL484 | IVC crRNA | TTAATACGACTCACTATAGGGACCAGGATGGGCACCA | |
| | | ACCGTTTTAGAGCTAGAAATAGCAAGTTAAAATAAGG | 135 |
| | | CTAGT | |
| BL485 | IVC crRNA | TTAATACGACTCACTATAGGGACCAGGATGGGCACCA | |
| | | GCCGTTTTAGAGCTAGAAATAGCAAGTTAAAATAAGG | 136 |
| | | CTAGT | |
| BL486 | IVC crRNA | TTAATACGACTCACTATAGGGACCAGGATGGGCACCA | |
| | | TCCGTTTTAGAGCTAGAAATAGCAAGTTAAAATAAGG | 137 |
| | | CTAGT | |
| BL318 | IVC crRNA | TTAATACGACTCACTATAGGGACCAGGATGGGCACCA | 138 |
| | | CCCGTTTTAGAGCTATGCTGTTTTG | |
| BL439 | IVC Conversion | AAAAGCACCGACTCGGTGCCACTTTTTCAAGTTGATA | 139 |
| | crRNA to | ACGGACTAGCCTTATTTTAACTTGCTATTTCTAGCTCT | |
| | sgRNA | AAAACGG | |
| BL472 | PCR sgRNA | AAGAGGAAGAGGTTAATACGACTCACTATAGGGAC | 140 |
| BL440 | PCR sgRNA | AAAAGCACCGACTCGGTGCC | 141 |
| BL473 | PCR sgRNA | ACTAGCCTTATTTTAACTTGCTATTTCTAGCTCTAAAA | 142 |
| | | CGG | |

Primers for UBP-containing Golden Gate inserts
italics denote the BsaI recognition sequence, underline denotes the BsaI restriction
site overhang sequence
insert primers for pUCX2 are also compatible with pBRX2 and pAIO GG plasmids
# denotes primers that were also used for biotin shift (the corresponding dedicated biotin
shift primers are identical in annealing sequence)
YZ401 and YZ403 are highly sensitive to annealing temperature. The optimal annealing
temperature is 54° C.

| | | | |
|---|---|---|---|
| BL528 | hEGFP insert for | AAGAAGGAGAAGGTCTCTAGTGGAGCAAGGGCGAGG | 143 |
| | pUCX2 GG fwd | AGCTGTTCACCG | |
| BL529 | hEGFP insert for | AAGAGAAGAGAGGTCTCATCGTGTTTACGTCGCCGTC | 144 |
| | pUCX2 GG rev | CAGCTC | |
| YZ148 | GFP66 insert for | ATGGGTCTCCAGTGGGGCCAACACTTGTCACTAC | 145 |
| | pUCX2 GG fwd | | |
| YZ149 | GFP66 insert for | ATGGGTCTCTTCGTTTCCGGATAACGGGAAAAGC | 146 |
| | pUCX2 GG rev | | |
| YZ150 | GFP151 insert | ATGGGTCTCCAGTGGCTCGAGTACAACTTTAACTCACA | 147 |
| | for pUCX2 GG | C | |
| | fwd | | |
| YZ151 | GFP151 insert | ATGGGTCTCTTCGTTGATTCCATTCTITTGTTTGTCTGC | 148 |
| | for pUCX2 GG | | |
| | rev | | |
| YZ97 | TK1 insert for | ATGGGTCTCTCATAGCTGTTTCCTGTGTGAAATTGTTA | 149 |
| | pUCX1 GG fwd | TCC | |
| YZ98 | TK1 insert for | ATGGGTCTCACCCCAGGCTTTACACTTTATGCTTCCG | 150 |
| | pUCX1 GG rev | | |
| YZ99^{#} | TK1 insert for | ATGGGTCTCCAGTGGCTGTTTCCTGTGTGAAATTGTTA | 151 |
| | pUCX2 GG fwd | TCC | |
| YZ100^{#} | TK1 insert for | ATGGGTCTCTTCGTTGGCTTTACACTTTATGCTTCCG | 152 |
| | pUCX2 GG rev | | |
| YZ118 | D8 insert for | ATGGGTCTCCAGTGGCACACAGGAAACAGCTATGAC | 153 |
| | pUCX2 GG fwd | | |
| YZ119 | D8 insert for | ATGGGTCTCTTCGTTGGGTTAAGCTTAACTTTAAGAAG | 154 |
| | pUCX2 GG rev | GAG | |
| YZ73^{#} | GFP151 insert | ATGGGTCTCACACAAACTCGAGTACAACTTTAACTCA | 155 |
| | for pAIO2X GG | CAC | |
| | fwd | | |
| YZ74^{#} | GFP151 insert | ATGGGTCTCGATTCCATTCTTTTGTTTGTCTGC | 156 |
| | for pAIO2X GG | | |
| | rev | | |
| YZ401^{#} | ESer insert for | ATTGGTCTCGGCCGAGCGGTTGAAGGCAC | 157 |
| | pAIO2X GG | | |
| | fwd | | |
| YZ403^{#} | ESer insert for | ATTGGTCTCTCTGGAACCCTTTCGGGTCG | 158 |
| | pAIO2X GG rev | | |

Biotin shift primers annealing temperature (° C.) denoted in parentheses

| | | | |
|---|---|---|---|
| BL745 | hEGFP fwd (48 | GGCGAGGAGCTGTTCACCG | 159 |
| | x 3 cycles, 54 x | | |
| | 20 cycles) | | |
| BL744 | hEGFP rev (48 | GTTTACGTCGCCGTCCAGCTC | 160 |
| | x 3 cycles, 54 x | | |
| | 20 cycles) | | |
| BL750 | GFP66 fwd (50) | GGCCAACACTTGTCACTACT | 161 |
| BL751 | GFP66 rev (50) | TCCGGATAACGGGAAAAGC | 162 |
| YZ351 | GFP151 fwd | CTCGAGTACAACTTTAACTCACAC | 163 |
| | (50) | | |
| YZ352 | GFP151 rev (50) | GATTCCATTCTTTTGTTTGTCTGC | 164 |
| BL748 | TK1 fwd (50) | CTGTTTCCTGTGTGAAATTGTTATCC | 165 |
| BL749 | TK1 rev (50) | GGCTTTACACTTTATGCTTCCG | 166 |
| BL774 | D8 fwd (50) | CCCGGGTTATTACATGCGCTAGCACT | 167 |
| BL775 | D8 rev (50) | GAAATTAATACGACTCACTATAGGGTTAAGCTTAACT | 168 |
| | | TTAAGAAGGAG | |
| YZ17 | pUCX2 fwd (60) | TGCAAGCAGCAGATTACGCGC | 169 |
| YZ18 | pUCX2 rev (60) | GTAACTGTCAGACCAAGTTTACTC | 170 |

UBP template oligonucleotides
*denotes sequences used in Cas9 experiments X denotes dNaM

| Name | Sequence Type | Sequence | |
|---|---|---|---|
| BL410* | hEGFP | GGCGAGGAGCTGTTCACCGGGXTGGTGCCCATCCTGG | 171 |
| | | TCGAGCTGGACGGCGACGTAAAC | |
| BL413* | hEGFP | GTTTACGTCGCCGTCCAGCTCGACCAGGATGGGXACC | 172 |
| | | ACCCCGGTGAACAGCTCCTCGCC | |
| BL411* | hEGFP | GTTTACGTCGCCGTCCAGCTCGACCAXGATGGGCACC | 173 |
| | | ACCCCGGTGAACAGCTCCTCGCC | |
| BL409* | hEGFP | GTTTACGTCGCCGTCCAGCTCGACCAGGATGGGCACC | 174 |
| | | AXCCCGGTGAACAGCTCCTCGCC | |
| TK1* | TK1 | CTGTTTCCTGTGTGAAATTGTTATCCGCTCACAXTTCC | 175 |
| | | ACACAACATACGAGCCGGAAGCATAAAGTGTAAAGC | |
| | | C | |
| DM510- | GFP151 | CTCGAGTACAACTTTAACTCACACAATGTAXAGATCA | 176 |
| 16* | | CGGCAGACAAACAAAAGAATGGAATC | |
| DM510- | GFP66 | CCGGATAACGGGAAAAGCATTGAACACCGCXGGTCA | 177 |
| 13* | | GAGTAGTGACAAGTGTTGGCCA | |
| DM510- | GFP66 | GGCCAACACTTGTCACTACTCTGACCXAGGGTGTTCA | 178 |
| 11* | | ATGCTTTTCCCGTTATCCGGA | |
| BL412* | hEGFP | GGCGAGGAGCTGTTCACCGGGGTGGTGCCCATCXTGG | 179 |
| | | TCGAGCTGGACGGCGACGTAAAC | |
| BL414* | hEGFP | GGCGAGGAGCTGTTCACCGGGGTGGTXCCCATCCTGG | 180 |
| | | TCGAGCTGGACGGCGACGTAAAC | |
| DM510- | GFP151 | GATTCCATTCTTTTGTTTGTCTGCCGTGATTXATACATT | 181 |
| 20* | | GTGTGAGTTAAAGTTGTACTCGAGT | |
| D8* | D8 | CACACAGGAAACAGCTATGACCCGGGTTATTACATGC | 182 |
| | | GCTAGCACTTGGAATTCACAATACTXTCTTTAAGGAA | |
| | | ACCATAGTAAATCTCCTTCTTAAAGTTAAGCTTAACCC | |
| | | TATAGTGAGTCGTATTAATTTC | |
| GFP151- | GFP151 | CTCGAGTACAACTTTAACTCACACAATGTAAXAATCA | 183 |
| 33 | | CGGCAGACAAACAAAAGAATGGAATC | |
| GFP151- | GFP151 | CTCGAGTACAACTTTAACTCACACAATGTAAXCATCA | 184 |
| 35 | | CGGCAGACAAACAAAAGAATGGAATC | |
| GFP151- | GFP151 | CTCGAGTACAACTTTAACTCACACAATGTAAXGATCA | 185 |
| 37 | | CGGCAGACAAACAAAAGAATGGAATC | |
| GFP151- | GFP151 | CTCGAGTACAACTTTAACTCACACAATGTCAXTGTCA | 186 |
| 39 | | CGGCAGACAAACAAAAGAATGGAATC | |
| GFP151- | GFP151 | CTCGAGTACAACTTTAACTCACACAATGTACXAATCA | 187 |
| 41 | | CGGCAGACAAACAAAAGAATGGAATC | |
| GFP151 | GFP151 | CTCGAGTACAACTTTAACTCACACAATGTACXCATCA | 188 |
| 43* | | CGGCAGACAAACAAAAGAATGGAATC | |
| DM510- | GFP151 | CTCGAGTACAACTTTAACTCACACAATGTACXGATCA | 89 |
| 19 | | CGGCAGACAAACAAAAGAATGGAATC | 1 |
| GFP151- | GFP151 | CTCGAGTACAACTTTAACTCACACAATGTACXTATCA | 190 |
| 47 | | CGGCAGACAAACAAAAGAATGGAATC | |
| GFP151- | GFP151 | CTCGAGTACAACTTTAACTCACACAATGTAGXAATCA | 191 |
| 49 | | CGGCAGACAAACAAAAGAATGGAATC | |
| GFP151- | GFP151 | CTCGAGTACAACTTTAACTCACACAATGTAGXCATCA | 192 |
| 51* | | CGGCAGACAAACAAAAGAATGGAATC | |
| GFP151- | GFP151 | CTCGAGTACAACTTTAACTCACACAATGTAGXGATCA | 193 |
| 53* | | CGGCAGACAAACAAAAGAATGGAATC | |
| GFP151- | GFP151 | CTCGAGTACAACTTTAACTCACACAATGTAGXTATCA | 194 |
| 55 | | CGGCAGACAAACAAAAGAATGGAATC | |
| GFP151- | GFP151 | CTCGAGTACAACTTTAACTCACACAATGTATXAATCA | 195 |
| 57 | | CGGCAGACAAACAAAAGAATGGAATC | |
| GFP151- | GFP151 | CTCGAGTACAACTTTAACTCACACAATGTATXCATCA | 196 |
| 59 | | CGGCAGACAAACAAAAGAATGGAATC | |
| GFP151- | GFP151 | CTCGAGTACAACTTTAACTCACACAATGTATXGATCA | 197 |
| 61* | | CGGCAGACAAACAAAAGAATGGAATC | |
| GFP151- | GFP151 | CTCGAGTACAACTTTAACTCACACAATGTATXTATCAC | 198 |
| 63 | | GGCAGACAAACAAAAGAATGGAATC | |
| GFP151- | GFP151 | CTCGAGTACAACTTTAACTCACACAATGTAAGXATCA | 199 |
| 69* | | CGGCAGACAAACAAAAGAATGGAATC | |
| ESer- | ESer | CTCTGGAACCCTTTCGGGTCGCCGGTTTAGXAGACCG | 200 |
| 69* | | GTGCCTTCAACCGCTCGGC | |

| Signal Peptide ID | Sequences | SEQ ID NO: |
|---|---|---|
| pelB-SP1 | MKYLLPTAEAGLLLLAAQPAIA | 225 |
| malE_SP2 | MKIKTGARILALSELTTMMFSASALA | 226 |
| phoA_SP3 | MKQSTIALALLPLLFTPVTKA | 227 |
| treA_SP4 | MKSPAPSRPQKMALIPACIFLCFAALSVQA | 228 |
| pcoE_SP5 | MKKILVSFVAIMAVASSAMA | 229 |
| Chitosanase | MKISMQKADFWKKAAISLLVFTMFFTLMMSETVFAAGL | 230 |
| (Csn)-SP6 | NK | |
| OmpA_SP7 | MKKTAIAIAVALAGFATVAQASAGLNKD | 231 |
| DsbAss | Protein: | 232 |
| | MKKIWLALAGLVLAFSASA | |
| | DNA: | 233 |
| | ATGAAAAAGATTTGGCTGGCGCTGGCTGGTTTAGTTTT | |
| | AGCGTTTAGCGCATCGGCG | |
| PelBss | Protein: | 234 |
| | MKYLLPTAAAGLLLLAAQPAMA | |
| | DNA: | 235 |
| | ATGAAATACCTGCTGCCGACCGCTGCTGCTGGTCTGCT | |
| | GCTCCTCGCTGCCCAGCCGGCGATGGCG | |
| PhoAss | Protein: | 227 |
| | MKQSTIALALLPLLFTPVTKA | |
| | DNA: | 236 |
| | ATGAAACAAAGCACTATTGCACTGGCACTCTTACCGT | |
| | TACTGTTTACCCCTGTGACAAAAGCG | |
| NTss | Protein: | 237 |
| | MKTHIVSSVTTTLLLGSILMNPVANA | |
| | DNA: | 238 |
| | ATGAAAACACATATAGTCAGCTCAGTAACAACAACAC | |
| | TATTGCTAGGTTCCATATTAATGAATCCTGTCGCTAAT | |
| | GCC | |
| NSP1 | Protein: | 239 |
| | MKYLLPWLALAGLVLAFSASA | |
| | DNA: | 240 |
| | ATGAAATACCTGCTGCCGTGGCTGGCGCTGGCTGGTT | |
| | TAGTTTTAGCGTTTAGCGCATCGGCG | |
| NSP2 | Protein: | 241 |
| | MKKITAAAGLLLLAAFSASA | |
| | DNA: | 242 |
| | ATGAAAAAGATTACCGCTGCTGCTGGTCTGCTGCTCCT | |
| | CGCTGCGTTTAGCGCATCGGCG | |
| NSP3 | Protein: | 243 |
| | MKKIWLALAGLVLAQPAMA | |
| | DNA: | 244 |
| | ATGAAAAAGATTTGGCTGGCGCTGGCTGGTTTAGTTTT | |
| | AGCCCAGCCGGCGATGGCG | |
| NSP3a | Protein: | 245 |
| | MKKILVLGALALWAQPAMA | |
| | DNA: | 246 |
| | ATGAAAAAGATTTTAGTTTTAGGTGCTCTGGCGCTGTG | |
| | GGCCCAGCCGGCGATGGCG | |
| NSP3b | Protein: | 247 |
| | MKKIWLALVLLAGAQPAMA | |
| | DNA: | 248 |
| | ATGAAAAAGATTTGGCTGGCGTTAGTTTTACTGGCTG | |
| | GTGCCCAGCCGGCGATGGCG | |
| NSP3c | Protein: | 249 |
| | MKKILAGWLALVLAQPAMA | |
| | DNA: | 250 |
| | ATGAAAAAGATTCTGGCTGGTTGGCTGGCGTTAGTTTT | |
| | AGCCCAGCCGGCGATGGCG | |
| NSP3d | Protein: | 251 |
| | MKKILVLLAGWLAAQPAMA | |
| | DNA: | 252 |
| | ATGAAAAAGATTTTAGTTTTACTGGCTGGTTGGCTGGC | |
| | GGCCCAGCCGGCGATGGCG | |
| NSP4 | Protein: | 253 |
| | MKKITAAAGLLLLAAQPAMA | |
| | DNA: | 254 |
| | ATGAAAAAGATTACCGCTGCTGCTGGTCTGCTGCTCCT | |
| | CGCTGCCCAGCCGGCGATGGCG | |
| NSP4a | Protein: | 255 |
| | MKKILLLLGTAAAAAQPAMA | |
| | DNA: | 256 |
| | ATGAAAAAGATTCTGCTGCTCCTCGGTACCGCTGCTG | |
| | CTGCTGCCCAGCCGGCGATGGCG | |
| NSP4b | Protein: | 257 |
| | MKKILLLLLLLLLLAQPAMA | |
| | DNA: | 258 |
| | ATGAAAAAGATTCTGCTGCTCCTCCTGCTGCTCCTCCT | |
| | GCTCGCCCAGCCGGCGATGGCG | |
| NSP4c | Protein: | 259 |
| | MKKIAAAAAAAAAAAQPAMA | |
| | DNA: | 260 |
| | ATGAAAAAGATTGCTGCTGCTGCTGCGGCGGCGGCGG | |
| | CTGCGGCCCAGCCGGCGATGGCG | |
| NSP5 | Protein: | 261 |
| | MKYLLPWLALAGLVLAQPAMA | |
| | DNA: | 262 |
| | ATGAAATACCTGCTGCCGTGGCTGGCGCTGGCTGGTT | |
| | TAGTTTTAGCCCAGCCGGCGATGGCG | |
| NSP6 | Protein: | 263 |
| | MKYLLPTAAAGLLLLAAFSASA | |
| | DNA: | 264 |
| | ATGAAATACCTGCTGCCGACCGCTGCTGCTGGTCTGCT | |
| | GCTCCTCGCTGCGTTTAGCGCATCGGCG | |

Table 3 illustrate signal peptide sequences described herein.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A method of increasing production of a nucleic acid molecule in a cell, comprising:
(a) expressing in the cell a recombinant nucleoside triphosphate transporter, wherein the recombinant nucleoside triphosphate transporter comprises an amino acid sequence comprising a deletion,
(b) incubating the cell with one or more unnatural nucleoside triphosphates, and
(c) transporting the one or more unnatural nucleoside triphosphates into the cell using the recombinant nucleoside triphosphate transporter.

2. The method of claim 1, wherein the nucleic acid molecule comprises DNA.

3. The method of claim 1, wherein the nucleic acid molecule comprises RNA.

4. The method of claim 1, wherein the recombinant nucleoside triphosphate transporter is from Phaeodactylum tricornutum.

5. The method of claim 1, wherein the deletion comprises about 5, 10, 15, 20, 22, 25, 30, 40, 44, 50, 60, 65, 66, 70, or more amino acid residues.

6. The method of claim 1, wherein the deletion is a N-terminal deletion.

7. The method of claim 1, wherein the amino acid sequence of the recombinant nucleoside triphosphate transporter is at least 80%, at least 85%, at least 90%, at least 95%, at least 99%, or at least 100% identical to SEQ ID NO: 4.

8. The method of claim 1, wherein the cell is an engineered cell.

9. The method of claim 8, wherein the engineered cell is E. coli.

10. The method of claim 1, wherein the cell is a prokaryotic cell.

11. The method of claim 10, wherein the cell is E. coli.

12. The method of claim 1, wherein the nucleic acid molecule comprises one or more unnatural nucleotides comprising a base selected from the group consisting of

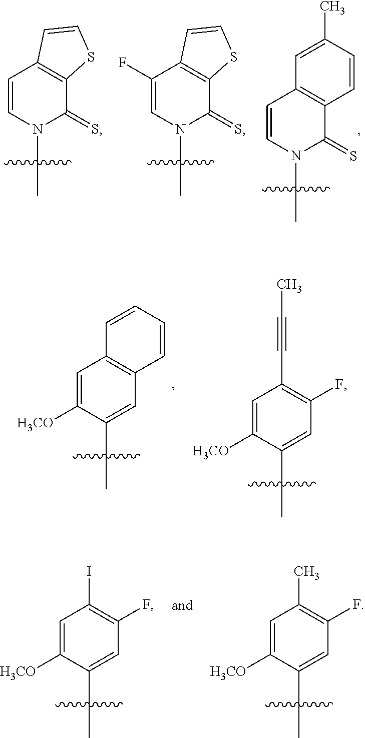

13. The method of claim 1, wherein the nucleic acid molecule comprises one or more unnatural nucleotides comprising a base which is

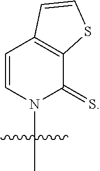 [SMILES: *n1ccc2ccsc2c1=S]

14. The method of claim 1, wherein the nucleic acid molecule comprises one or more unnatural nucleotides comprising a base which is

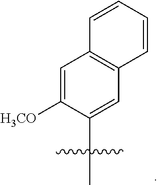 [SMILES: *c1cc2ccccc2cc1OC]

15. The method of claim 1, wherein the one or more unnatural nucleoside triphosphates comprises a base selected from the group consisting of

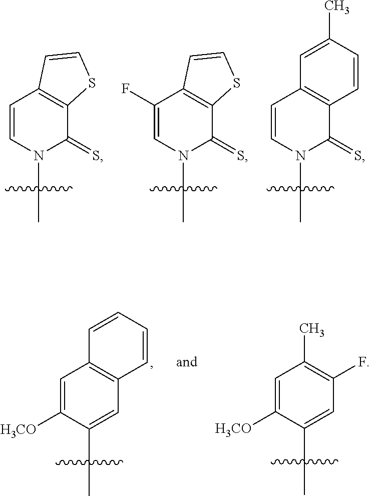

16. The method of claim 1, wherein the one or more unnatural nucleoside triphosphates comprises a base which is

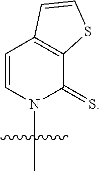 [SMILES: *n1ccc2ccsc2c1=S]

17. The method of claim 1, wherein the one or more unnatural nucleoside triphosphates comprises a base which is

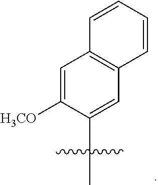 [SMILES: *c1cc2ccccc2cc1OC]

18. A method of increasing production of a DNA nucleic acid molecule in a cell comprising:
(a) expressing in the cell a recombinant nucleoside triphosphate transporter, wherein the recombinant nucleoside triphosphate transporter comprises an amino acid sequence comprising a deletion,
(b) incubating the cell with one or more unnatural 2′-deoxyribonucleoside triphosphates, and
(c) transporting the one or more unnatural 2′-deoxyribonucleoside triphosphates into the cell using the recombinant nucleoside triphosphate transporter, wherein the one or more unnatural 2′-deoxyribonucleoside triphosphates comprise a base selected from the group consisting of:

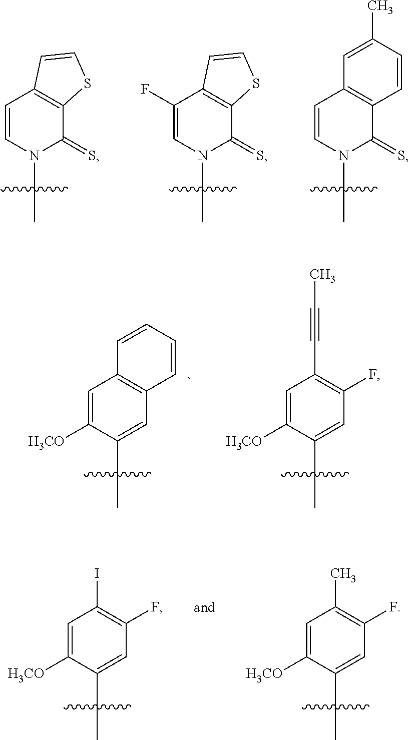

19. The method of claim 18, wherein the cell is E. coli.

20. The method of claim 18, wherein the recombinant nucleoside triphosphate transporter is from Phaeodactylum tricornutum.

21. The method of claim 18, wherein the deletion comprises about 5, 10, 15, 20, 22, 25, 30, 40, 44, 50, 60, 65, 66, 70, or more amino acid residues.

22. The method of claim 18, wherein the deletion is a N-terminal deletion.

23. The method of claim 18, wherein the amino acid sequence of the recombinant nucleoside triphosphate transporter is at least 80%, at least 85%, at least 90%, at least 95%, at least 99%, or at least 100% identical to SEQ ID NO: 4.

24. A method of increasing production of a RNA nucleic acid molecule in a cell, comprising:
(a) expressing in the cell a recombinant nucleoside triphosphate transporter, wherein the recombinant nucleoside triphosphate transporter comprises an amino acid sequence comprising a deletion,
(b) incubating the cell with one or more unnatural ribonucleoside triphosphates, and
(c) transporting the one or more unnatural ribonucleoside triphosphates into the cell using the recombinant nucleoside triphosphate transporter, wherein the one or more unnatural ribonucleoside triphosphates comprise a base selected from the group consisting of:

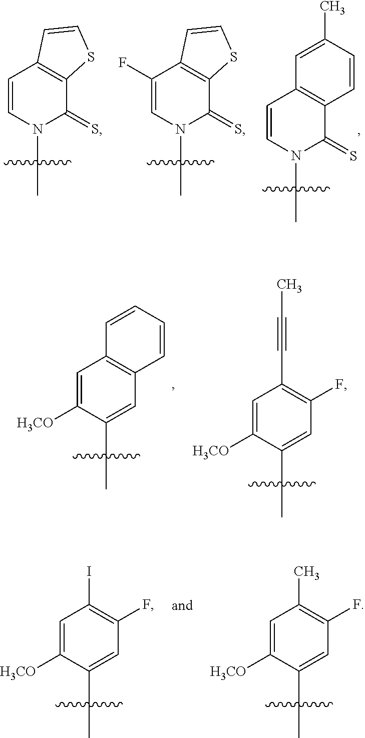

25. The method of claim 24, wherein the cell is E. coli.

26. The method of claim 24, wherein the recombinant nucleoside triphosphate transporter is from Phaeodactylum tricornutum.

27. The method of claim 24, wherein the deletion comprises about 5, 10, 15, 20, 22, 25, 30, 40, 44, 50, 60, 65, 66, 70, or more amino acid residues.

28. The method of claim 24, wherein the deletion is a N-terminal deletion.

29. The method of claim 24, wherein the amino acid sequence of the recombinant nucleoside triphosphate transporter is at least 80%, at least 85%, at least 90%, at least 95%, at least 99%, or at least 100% identical to SEQ ID NO: 4.
